(19) United States
(12) Patent Application Publication
(10) Pub. No.: US 2026/0218198 A1
(43) Pub. Date: Jul. 30, 2026

(54) NOVEL FAS RNAi THERAPEUTICS AND USES THEREOF

(71) Applicant: ELI LILLY AND COMPANY, Indianapolis, IN (US)

(72) Inventors: Patrick Joseph ANTONELLIS, Norwell, MA (US); Seth Andrew BAWEL, Brownsburg, IN (US); Matthew Joseph HAMANG, Indianapolis, IN (US); Theodore Curtis JESSOP, Zionsville, IN (US); Gregory Lawrence LACKNER, Indianapolis, IN (US); Jibo WANG, Carmel, IN (US); Takako WILSON, San Diego, CA (US)

(21) Appl. No.: 19/141,294

(22) PCT Filed: Dec. 20, 2023
(86) PCT No.: PCT/US23/84990

§ 371 Date: Jun. 19, 2025

Related U.S. Application Data
(60) Provisional application No. 63/476,483, filed on Dec. 21, 2022.

(51) Int. Cl. C12N 15/113; A61P 1/16

(52) U.S. Cl. CPC C12N 15/1138; A61P 1/16; C12N 2310/14; C12N 2310/315; C12N 2310/321; C12N 2310/322; C12N 2310/351

(57) ABSTRACT

The present invention relates to novel therapeutic compounds, known as RNAi agents, that decrease expression of the FAS receptor (expressed by the FAS gene), thereby decreasing expression of FAS mRNA and protein expression. Such RNAi agents are useful in the treatment of diseases involving the regulation of FAS expression and function, such as autoimmune hepatitis.

### BACKGROUND

The present invention relates to novel therapeutic compounds, known as RNAi agents, that decrease expression of the FAS (expressed by the FAS gene), thereby decreasing expression of FAS mRNA and FAS protein. Such RNAi agents are useful in the treatment of diseases involving the regulation of FAS expression and function, such as autoimmune hepatitis.

FAS, the Fas cell death receptor, and its ligand, FASL, are members of the TNFR superfamily. Binding of FASL to FAS results in downstream death-inducing signaling involving caspases (e.g., caspase 8 and 10) and Fas-associated death domain protein (FADD), which form a complex. Caspase autoproteolysis in the complex results in caspase cascade, and leads to apoptosis. NF-kappaB, MAPK3/ERK1, and MAPK8/JNK are also known to be activated by FAS signaling, and such activation is thought to result in proliferation in normal diploid fibroblast and T cells. These play an important role in regulation of the immune response involving cells that express FAS, which includes hepatocytes.

Autoimmune hepatitis (AIH) is a chronic inflammation of the liver with no identifiable cause such as a viral infection. AIH patients have increased FAS levels in hepatocytes. Genetics, the environment (such as an environmental trigger), and native immune system dysregulation are thought to play a part in the progression of the disease from inflammation to liver fibrosis. AIH often first presents as patients reach their teen years.

Treatment options are limited and include high dose steroid treatment, often in combination with azathioprine, another immunosuppressive agent. Treatment is correlated with a downregulation of FAS and patients can achieve near remission of inflammation biochemically. However, steroid treatment, especially when taken long term and/or in high doses, can cause a wide range of serious side effects, as can other immunosuppressive agent treatment such azathioprine. Serious side effects can include onset of diabetes, thinning bones (osteoporosis), broken bones (osteonecrosis), high blood pressure, cataracts, glaucoma, and weight gain. If treatment is removed, patients often experience recurrence, and some patients experience disease progression that requires a liver transplant. Accordingly, there is a need for improved treatments for AIH.

### SUMMARY OF INVENTION

In one aspect, provided herein are RNAi agents for reducing FAS gene expression, wherein the RNAi agent comprises a delivery moiety of Formula I conjugated to R, wherein R is a double stranded RNA (dsRNA) comprising an antisense strand and a sense strand:

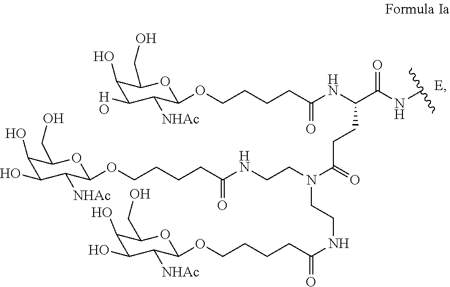

wherein R is conjugated to connection point E of Formula I, optionally via a linker, wherein the sense strand and the antisense strand form a duplex region, and wherein the antisense strand comprises a region of complementarity to a FAS mRNA target sequence of SEQ ID NO: 1, and wherein the sense and antisense strand each optionally comprise one or more modified nucleotides and one or more modified internucleotide linkages. In some embodiments, Formula I is conjugated to the sense strand, optionally via a linker. In some embodiments, Formula I is conjugated to the 3′ terminal nucleotide of the sense strand, optionally via a linker.

In some embodiments, the antisense strand is 15 to 50 nucleotides in length. In some embodiments, the sense strand is 15 to 50 nucleotides in length. In some embodiments, the antisense strand is between 18 and 23 nucleotides in length. In some embodiments, the sense strand is between 18 and 21 nucleotides in length. In some embodiments, the antisense strand is 23 nucleotides in length and the sense strand is 21 nucleotides in length.

In some embodiments, the sense strand or the antisense strand comprises a sequence selected from Table 2, 3A, 3B, 4A, 4B, 7, or 8 disclosed herein. In some embodiments, the sense strand and the antisense strand comprises a sequence selected from Table 2, 3A, 3B, 4A, 4B, 7, or 8 disclosed herein.

In some embodiments, R is conjugated to Formula I via a linker. In further embodiments, the linker comprises a linker of Formula II having connection points A and B or the linker comprises Formula III having connection points C and D, and wherein:

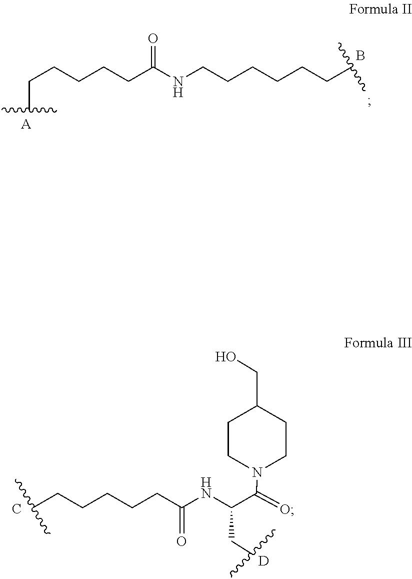

a. Formula I conjugated to Formula II at connection point A and Formula II is conjugated to a phosphate group at connection point B, and the phosphate group is further conjugated to R; or
b. Formula I conjugated to Formula III at connection point C and Formula III is conjugated to a phosphate group at connection point D, and the phosphate group is further conjugated to R.

In another aspect, provided herein are pharmaceutical composition comprising the FAS RNAi agent described herein and one or more pharmaceutically acceptable excipients.

In another aspect, provided herein are methods of treating autoimmune hepatitis (AIH) in a patient in need thereof, comprising administering to the patient a FAS RNAi agent or pharmaceutical composition thereof described herein.

In another aspect, provided herein are FAS RNAi agent for use in a therapy. Also provided herein are FAS RNAi agent for use in in the treatment of AIH. Also provided herein are uses of FAS RNAi agent in the manufacture of a medicament for the treatment of AIH

### DETAILED DESCRIPTION

FAS siRNAs and ASOs have been described, but none have progressed for treatment in patients, including for the treatment of AIH. Using the FAS RNAi agents herein to decrease expression of FAS can be employed to treat AIH in patients in need thereof. Such siRNAs may exhibit one or more of, e.g., as compared to other liver targeted siRNAs such as FAS siRNAs comprising a different delivery ligand, a different sequence, a differently modified sequence, or as compared to treatment with a vehicle control: improved knockdown in the liver; improved tissue exposure, improved exposure in liver hepatocytes; an improved durable response; an improved pharmacokinetic profile; fewer off target effects; and/or an improved toxicity profile. Other embodiments of the FAS RNAi agents herein may include one or more of fewer side effects as compared to steroids or other standard of care; an improved toxicity profile; an improved safety profile; improved tolerability or compliance; and/or improved liver function tests. Still other siRNAs herein may have other benefits, e.g., in combination with any of the preceding or as a stand-alone benefit, including improved and/or simplified synthesis, synthetic processes with fewer degradation products; or any combination thereof.

The RNAi agents herein comprise a sense strand and an antisense strand, wherein each is an oligonucleotide. In some embodiments, the RNAi agent described herein also comprise a delivery moiety. As used herein, “nucleotide” means an organic compound having a nucleoside (a nucleobase such as, for example, adenine, cytosine, guanine, thymine, or uracil; and a pentose sugar such as, for example, ribose or 2′-deoxyribose) and a phosphate group. A “nucleotide” can serve as a monomeric unit of nucleic acid polymers such as deoxyribonucleic acid (DNA) and ribonucleic acid (RNA).

As used herein, “oligonucleotide” means a short nucleic acid compound (e.g., less than about 100 nucleotides in length). An oligonucleotide may be single-stranded (ss) or double stranded (ds). An oligonucleotide may or may not have duplex regions. As a set of non-limiting examples, an oligonucleotide may be, but is not limited to, a small interfering RNA (siRNA), microRNA (miRNA), short hairpin RNA (shRNA), Dicer substrate interfering RNA (DsiRNA), or antisense oligonucleotide (ASO).

As used herein, “ribonucleotide” means a nucleotide having a ribose as its pentose sugar, which contains a hydroxyl group at its 2′ position. A modified ribonucleotide is a ribonucleotide having one or more modifications or substitutions of atoms other than hydrogen at the 2′ position, including modifications or substitutions in or of the nucleobase, sugar, or phosphate group.

As used herein, “modified internucleotide linkage” means an internucleotide linkage having one or more chemical modifications when compared with a reference internucleotide linkage having a phosphodiester bond. A modified internucleotide linkage can be a non-naturally occurring linkage.

As used herein, “modified nucleotide” refers to a nucleotide having one or more chemical modifications when compared with a corresponding reference nucleotide selected from: adenine ribonucleotide, guanine ribonucleotide, cytosine ribonucleotide, uracil ribonucleotide, adenine deoxyribonucleotide, guanine deoxyribonucleotide, cytosine deoxyribonucleotide, and thymidine deoxyribonucleotide. A modified nucleotide can be a non-naturally occurring nucleotide. A modified nucleotide can have, for example, one or more chemical modification in its sugar, nucleobase, and/or phosphate group. Additionally, or alternatively, a modified nucleotide can have one or more chemical moieties conjugated to a corresponding reference nucleotide.

The term “percentage sequence identity” with respect to a reference nucleic acid sequence is defined as the percentage of nucleotides, nucleosides, or nucleobases in a candidate sequence that are identical with the nucleotides, nucleosides, or nucleobases in the reference nucleic acid sequence, after optimally aligning the sequences and introducing gaps or overhangs, if necessary, to achieve the maximum percent sequence identity. Alignment for purposes of determining percent nucleic acid sequence identity can be achieved in various ways that are within the skill in the art, for instance, using publicly available computer software programs, for example, those described in Current Protocols in Molecular Biology (Ausubel et al., eds., 1987, Supp. 30, section 7.7.18, Table 7.7.1), and including BLAST, BLAST-2, ALIGN, Clustal W2.0 or Clustal X2.0 or Megalign (DNASTAR) software. In one embodiment herein, sequence identity is calculated use Clustal W2.0 or Clustal X2.0. In another embodiment, sequence identity is calculated using Clustal W2.0. In another embodiment, sequence identity is calculated using Clustal X2.0. Those skilled in the art can determine appropriate parameters for measuring alignment, including any algorithms needed to achieve maximal alignment over the full length of the sequences being compared. Percentage of “sequence identity” can be determined by comparing two optimally aligned sequences over a comparison window, where the fragment of the nucleic acid sequence in the comparison window may comprise additions or deletions (e.g., gaps or overhangs) as compared to the reference sequence (which does not comprise additions or deletions) for optimal alignment of the two sequences. The percentage can be calculated by determining the number of positions at which the identical nucleotide, nucleoside, or nucleobase occurs in both sequences to yield the number of matched positions, dividing the number of matched positions by the total number of positions in the window of comparison, and multiplying the result by 100 to yield the percentage of sequence identity. The output is the percent identity of the subject sequence with respect to the query sequence. In some embodiments, percent sequence identity is the percent of nucleotide residues that are identical between two strands using the PID3 calculation, which is the number of identical nucleotide residues divided by the total number of nucleotides of the shortest of the two sequences, multiplied by 100. See, e.g., Raghava, G., Barton, G. J. Quantification of the variation in percentage identity for protein sequence alignments. BMC Bioinformatics 7, 415 (2006).

As used herein, “phosphate analog” means a chemical moiety that mimics the electrostatic and/or steric properties of a phosphate group. In some embodiments, a phosphate analog is positioned at the 5′ terminal nucleotide of an oligonucleotide in place of a 5′-phosphate. A 5′ phosphate analog can include a phosphatase-resistant linkage. Examples of phosphate analogs include, but are not limited to, 5′ phosphonates, such as 5′ methylene phosphonate (5′-MP) and 5′-(E)-vinylphosphonate (5′-VP). An oligonucleotide can have a phosphate analog at a 4′-carbon position of the sugar (referred to as a “4′-phosphate analog”) at a 5′-terminal nucleotide. An example of a 4′-phosphate analog is oxymethylphosphonate, in which the oxygen atom of the oxymethyl group is bound to the sugar moiety (e.g., at its 4′-carbon) or analog thereof. See, e.g., Intl. Patent Application Publication No. WO 2018/045317. Other modifications have been developed for the 5′ end of oligonucleotides (see, e.g., Intl. Patent Application No. WO 2011/133871; U.S. Pat. No. 8,927,513; and Prakash et al. (2015) Nuc. Acids Res. 43:2993-3011).

As used herein, “region of complementarity” means a nucleotide sequence of a nucleic acid (e.g., a double stranded oligonucleotide) that is sufficiently complementary to an antiparallel nucleotide sequence to permit hybridization between the two sequences of nucleotides under appropriate hybridization conditions (e.g., in a phosphate buffer, in a cell, etc.). In some embodiments, an oligonucleotide herein includes a targeting sequence having a region of complementary to a mRNA target sequence.

As used herein, “duplex,” in reference to nucleic acids or oligonucleotides, such as a sense strand or an antisense strand means a structure formed through hydrogen bonds of complementary base pairing of two antiparallel sequences of nucleotides under suitable conditions to promote such a structure. A duplex may form despite not having full complementarity between the two strands, or when an abasic nucleotide is present.

RNA interference is a specialized cellular process that utilizes RISC for degrading RNA in a sequence dependent manner. As used herein, “RNAi agent” means an agent comprising either (a) a double stranded oligonucleotide having a sense strand (passenger) and antisense strand (guide), in which the antisense strand or part of the antisense strand is used by the Argonaute 2 (Ago2) endonuclease in the cleavage of a target mRNA or (b) a single stranded oligonucleotide having a single antisense strand, where that antisense strand (or part of that antisense strand) is used by the Ago2 endonuclease in the cleavage of a target mRNA. In some embodiments, the RNAi agent described herein also comprise a delivery moiety.

As used herein, “treatment” or “treating” refers to all processes wherein there may be a slowing, controlling, delaying, or stopping of the progression of the disorders or disease disclosed herein, or ameliorating disorder or disease symptoms, and need not indicate a total elimination of all disorder or disease symptoms. Treatment includes administration of an RNAi agent or pharmaceutical composition thereof for treatment of a disease or condition in a mammal including a human.

An “effective amount” refers to an amount necessary (for periods of time and for the means of administration) to achieve the desired therapeutic result. An effective amount of a RNAi agent may vary according to factors such as the disease state, age, sex, and weight of the individual, and the ability of the RNAi agent to elicit a desired response in the individual. An effective amount is also one in which any toxic or detrimental effects of the RNAi agent are outweighed by the therapeutically beneficial effects.

Provided herein are RNAi agents for reducing FAS gene expression, wherein the RNAi agent comprises a delivery moiety of Formula I conjugated to R, wherein R is a double stranded RNA (dsRNA) comprising an antisense strand and a sense strand:

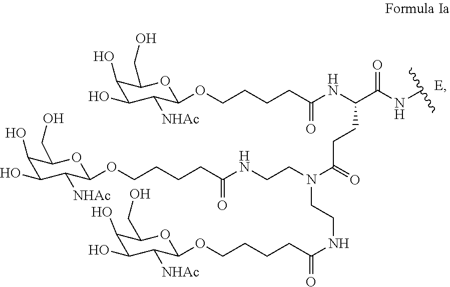

wherein R is conjugated to connection point E of Formula I, optionally via a linker, wherein the sense strand and the antisense strand form a duplex region, and wherein the antisense strand comprises a region of complementarity to a FAS mRNA target sequence of SEQ ID NO: 1, and wherein the sense and antisense strand each optionally comprise one or more modified nucleotides and one or more modified internucleotide linkages.

Also provided here are RNAi agents for reducing FAS gene expression, wherein the RNAi agent comprises a delivery moiety of Formula Ia conjugated to R, wherein R comprises an antisense strand and a sense strand:

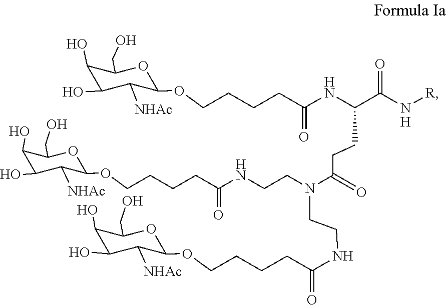

wherein R is optionally conjugated to Formula Ia via a linker, wherein the sense strand and the antisense strand form a duplex region, and wherein the antisense strand comprises a region of complementarity to a FAS mRNA target sequence of SEQ ID NO: 1, and wherein the sense and antisense strand each optionally comprise one or more modified nucleotides and one or more modified internucleotide linkages.

Disclosed herein are RNAi agents for reducing FAS gene expression, wherein the RNAi agents comprise a dsRNA comprising a sense strand and an antisense strand, wherein the sense strand and the antisense strand form a duplex region, and wherein the antisense strand comprises a region of complementarity of at least 15 nucleotides to the sequence as set forth in SEQ ID NO: 1, and wherein the sense strand and/or the antisense strand each optionally comprise one or more modified nucleotides and/or modified internucleotide linkages. In further embodiments, the antisense strand comprises at least 15 nucleotides of a sequence in Table 2. In further embodiments, the antisense strand comprises at least 18 nucleotides of a sequence in Table 2. In further embodiments, the RNAi agent reduces FAS gene expression by about 50% or greater in a cell expressing FAS, as compared to a control. In further embodiments, the RNAi agent reduces FAS gene expression by reducing the level of FAS mRNA transcript, the level of FAS protein, or both.

In further embodiments, the antisense strand is 15 to 50 nucleotides in length, and/or the sense strand is 15 to 50 nucleotides in length. In further embodiments, the sense and/or sense strand is independently 15 to 30 nucleotides in length. In further embodiments, the antisense strand is between 18 and 23 nucleotides in length. In further embodiments, the sense strand is between 18 and 21 nucleotides in length.

In further embodiments, the RNAi agent comprises an antisense strand that comprises at least 15 contiguous nucleotides of a sequence selected from the group consisting of SEQ ID NOs: 2-112. In still further embodiments, the antisense strand comprises at least 18 contiguous nucleotides of a sequence selected from the group consisting of SEQ ID NOs: 2-112.

In other further embodiments, the antisense strand comprises at least 18 contiguous nucleotides of a sequence selected from the group consisting of SEQ ID NOs: 224 to 334, 337, 338, 573, and 577.

In other further embodiments, the sense strand comprises at least 18 contiguous nucleotides of a sequence selected from the group consisting of SEQ ID NOs: 113 to 223, 335, 336, 572, and 576.

In further embodiments, the antisense strand of the RNAi agent is 23 nucleotides in length. In still further embodiments, the sense strand is 21 nucleotides in length. In another embodiment, the sense and antisense strand comprise a sequence selected from the sequences set forth in Table 3A.

The sense strand and the antisense strand of the RNAi agents disclosed herein do not require full complementarity. Accordingly, in the RNAi agents disclosed herein, the duplex region between the sense strand and the antisense strand comprises 0, 1, 2, or 3 mismatches between the sense strand and the antisense strand. In further embodiments, the duplex region between the sense strand and the antisense strand consists of 0, 1, 2, or 3 mismatches between the sense strand and the antisense strand.

In some embodiments, the RNAi agent comprises a sense strand comprising a first nucleic acid sequence, and an antisense strand comprises a second nucleic acid sequence, wherein the first nucleic acid sequence and the second nucleic acid sequence are selected from the group consisting of:
a. the first nucleic acid sequence has at least 90% sequence identity to SEQ ID NO: 129, and the second nucleic acid sequence has at least 90% sequence identity to SEQ ID NO: 240;
b. the first nucleic acid sequence has at least 90% sequence identity to SEQ ID NO: 116, and the second nucleic acid sequence has at least 90% sequence identity to SEQ ID NO: 227;
c. the first nucleic acid sequence has at least 90% sequence identity to SEQ ID NO: 151, and the second nucleic acid sequence has at least 90% sequence identity to SEQ ID NO: 262;
d. the first nucleic acid sequence has at least 90% sequence identity to SEQ ID NO: 128, and the second nucleic acid sequence has at least 90% sequence identity to SEQ ID NO: 239; and
e. the first nucleic acid sequence has at least 90% sequence identity to SEQ ID NO: 155, and the second nucleic acid sequence has at least 90% sequence identity to SEQ ID NO: 266.

In some embodiments, the RNAi agent comprises a sense strand comprising a first nucleic acid sequence, and an antisense strand comprises a second nucleic acid sequence, wherein the first nucleic acid sequence and the second nucleic acid sequence are selected from the group consisting of:
a. the first nucleic acid sequence has at least 95% sequence identity to SEQ ID NO: 129, and the second nucleic acid sequence has at least 95% sequence identity to SEQ ID NO: 240;
b. the first nucleic acid sequence has at least 95% sequence identity to SEQ ID NO: 116, and the second nucleic acid sequence has at least 95% sequence identity to SEQ ID NO: 227;
c. the first nucleic acid sequence has at least 95% sequence identity to SEQ ID NO: 151, and the second nucleic acid sequence has at least 95% sequence identity to SEQ ID NO: 262;
d. the first nucleic acid sequence has at least 95% sequence identity to SEQ ID NO: 128, and the second nucleic acid sequence has at least 95% sequence identity to SEQ ID NO: 239; and
e. the first nucleic acid sequence has at least 95% sequence identity to SEQ ID NO: 155, and the second nucleic acid sequence has at least 95% sequence identity to SEQ ID NO: 266.

In some embodiments, the RNAi agent comprises a sense strand comprising a first nucleic acid sequence, and an antisense strand comprises a second nucleic acid sequence, wherein the first nucleic acid sequence and the second nucleic acid sequence are selected from the group consisting of:
a. the first nucleic acid sequence comprises SEQ ID NO: 129, and the second nucleic acid sequence comprises SEQ ID NO: 240;
b. the first nucleic acid sequence comprises SEQ ID NO: 116, and the second nucleic acid sequence comprises SEQ ID NO: 227;
c. the first nucleic acid sequence comprises SEQ ID NO: 151, and the second nucleic acid sequence comprises SEQ ID NO: 262;
d. the first nucleic acid sequence comprises SEQ ID NO: 128, and the second nucleic acid sequence comprises SEQ ID NO: 239; and
e. the first nucleic acid sequence comprises SEQ ID NO: 155, and the second nucleic acid sequence comprises SEQ ID NO: 266.

In further embodiments, the sense strand and the antisense strand each independently comprise one or more modified nucleotides, such as 2′ fluoro modified nucleotides or 2′-O-methyl modified nucleotides. In still further embodiments of the RNAi agents disclosed herein, each nucleotide of the sense strand and each nucleotide of the antisense strand is a modified nucleotide. In further embodiments, each nucleotide is a 2′ fluoro modified nucleotide or a 2′-O-methyl modified nucleotide.

In further embodiments of the RNAi agents disclosed herein, the antisense strand is 23 nucleotides in length, each nucleotide of the antisense strand is a modified nucleotide, and 2′ fluoro modified nucleotides are present at
a. Positions 2, 3, 7, 14, and 16 from the 5′ end of the antisense strand; or
b. Positions 2, 5, 7, 14, and 16 from the 5′ end of the antisense strand; or
c. Positions 2, 3, 8, 14, and 16 from the 5′ end of the antisense strand; or
d. Positions 2, 5, 8, 14, and 16 from the 5′ end of the antisense strand; or
e. Positions 2, 6, 14, and 16 from the 5′ end of the antisense strand.

In further embodiments, the nucleotides that are not 2′ fluoro modified nucleotides are 2′-O-methyl modified nucleotides.

In further embodiments of the RNAi agents disclosed herein, the sense strand and antisense strand each independently comprise one or more modified internucleotide linkages, and each modified internucleotide linkage is a phosphorothioate linkage. In further embodiments, the sense strand and antisense strand each independently comprise four phosphorothioate linkages. In still further embodiments, the two terminal nucleotides at each of the 5′ and 3′ ends of each of the sense and antisense strand are phosphorothioate linkages.

In other embodiments, the 5′ nucleotide of the antisense strand comprises a phosphate group or a phosphate analog. As used herein, “phosphate analog” means a chemical moiety that mimics the electrostatic and/or steric properties of a phosphate group. In some embodiments, a phosphate analog is positioned at the 5′ terminal nucleotide of an oligonucleotide in place of a 5′-phosphate. A 5′ phosphate analog can include a phosphatase-resistant linkage. Examples of phosphate analogs include, but are not limited to, 5′ phosphonates, such as 5′ methylene phosphonate (5′-MP) and 5′-(E)-vinylphosphonate (5′-VP). An oligonucleotide can have a phosphate analog at a 4′-carbon position of the sugar (referred to as a “4′-phosphate analog”) at a 5′-terminal nucleotide. An example of a 4′-phosphate analog is oxymethylphosphonate, in which the oxygen atom of the oxymethyl group is bound to the sugar moiety (e.g., at its 4′-carbon) or analog thereof. See, e.g., Intl. Patent Application Publication No. WO 2018/045317. Other modifications have been developed for the 5′ end of oligonucleotides (see, e.g., Intl. Patent Application No. WO 2011/133871; U.S. Pat. No. 8,927,513; and Prakash et al. (2015) Nuc. Acids Res. 43:2993-3011).

In further embodiments of the RNAi agents disclosed herein, the antisense strand comprises a sequence selected from the group consisting of SEQ ID NOs: 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 563, 566, 567, 569, 570, 571, 575, 579, 581, 583, 585, or a sequence having at least 90% sequence identity thereto, wherein the 5′ terminal nucleotide of the antisense strand comprises a vinyl phosphonate, a phosphate, or a hydroxyl group. In other embodiments, the phosphate group listed at the 5′ end of the recited SEQ ID NO: is removed and replaced with an OH. In other embodiments, the phosphate group listed at the 5′ end of the recited SEQ ID NO: is replaced with a 5′ vinylphosphonate.

In further embodiments of the RNAi agents disclosed herein, the antisense strand comprises a sequence selected from the group consisting of SEQ ID NOs: 587, 589, 591, 593, 595, 597, 599, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 663, 665, 667, 669, 671, 673, 675, 677, 679, 681, 683, 685, 687, 689, 691, 693, 695, 697, 699, 701, 703, 705, 707, 709, 711, 713, 715, 717, 719, 721, 723, 725, 727, 729, 731, 733, 735, 737, 739, 741, 743, 745, 747, 749, 751, 753, 755, 757, 759, 761, 763, 765, 767, 769, 771, 773, 775, 777, 779, 781, 783, 785, 787, 789, 791, 793, 795, 797, 799, 801, 803, 805, 807, 813, 815, 817, 819, or a sequence having at least 90% sequence identity thereto.

In further embodiments the sense strand comprises a sequence selected from the group consisting of SEQ ID NOs: 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 564, 565, 568, 574, 578, 580, 582, 584, or a sequence having at least 90% sequence identity thereto.

In further embodiments the sense strand comprises a sequence selected from the group consisting of SEQ ID NOs: 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 678, 680, 682, 684, 686, 688, 690, 692, 694, 696, 698, 700, 702, 704, 706, 708, 710, 712, 714, 716, 718, 720, 722, 724, 726, 728, 730, 732, 734, 736, 738, 740, 742, 744, 746, 748, 750, 752, 754, 756, 758, 760, 762, 764, 766, 768, 770, 772, 774, 776, 778, 780, 782, 784, 786, 788, 790, 792, 794, 796, 798, 800, 802, 804, 806, 808, 809, 810, 811, 812, 814, 816, 818, or a sequence having at least 90% sequence identity thereto.

In still further embodiments of the RNAi agents disclosed herein, the sense strand and antisense strand are a pair of oligonucleotide sequences selected from Table 4A or 4B, or a sequence that is at least 80, 81, 82, 83, 84, 85, 86, 87, 88, 89, 90, 91, 92, 93, 94, 95, 96, 97, 98, or 99 percent identical to the sequence in Table 4A or 4B. In further embodiments, 1, 2, or 3 mismatches are introduced into the sense strand of the pair in Table 4A, Table 4B or Table 7. In further embodiments, 1, 2, or both terminal nucleotides of 5′ end of the antisense strand are changed.

In some embodiments, the RNAi agent comprises a sense strand comprising a first nucleic acid sequence, and an antisense strand comprises a second nucleic acid sequence, wherein the first nucleic acid sequence and the second nucleic acid sequence are selected from the group consisting of:
a. the first nucleic acid sequence has at least 95% sequence identity to SEQ ID NO: 339, and the second nucleic acid sequence has at least 95% sequence identity to SEQ ID NO: 340;
b. the first nucleic acid sequence has at least 95% sequence identity to SEQ ID NO: 341, and the second nucleic acid sequence has at least 95% sequence identity to SEQ ID NO: 342;
c. the first nucleic acid sequence has at least 95% sequence identity to SEQ ID NO: 343, and the second nucleic acid sequence has at least 95% sequence identity to SEQ ID NO: 344;
d. the first nucleic acid sequence has at least 95% sequence identity to SEQ ID NO:345, and the second nucleic acid sequence has at least 95% sequence identity to SEQ ID NO: 346;
e. the first nucleic acid sequence has at least 95% sequence identity to SEQ ID NO: 347, and the second nucleic acid sequence has at least 95% sequence identity to SEQ ID NO: 348;
f. the first nucleic acid sequence has at least 95% sequence identity to SEQ ID NO: 349, and the second nucleic acid sequence has at least 95% sequence identity to SEQ ID NO: 350;
g. the first nucleic acid sequence has at least 95% sequence identity to SEQ ID NO: 353, and the second nucleic acid sequence has at least 95% sequence identity to SEQ ID NO: 354;
h. the first nucleic acid sequence has at least 95% sequence identity to SEQ ID NO: 363 and the second nucleic acid sequence has at least 95% sequence identity to SEQ ID NO: 364; and
i. the first nucleic acid sequence has at least 95% sequence identity to SEQ ID NO: 381, and the second nucleic acid sequence has at least 95% sequence identity to SEQ ID NO: 382.

In some embodiments, the RNAi agent comprises a sense strand comprising a first nucleic acid sequence, and an antisense strand comprises a second nucleic acid sequence, wherein the first nucleic acid sequence and the second nucleic acid sequence are selected from the group consisting of:
a. the first nucleic acid sequence has at least 90% sequence identity to SEQ ID NO: 564 or 809, and the second nucleic acid sequence has at least 90% sequence identity to SEQ ID NO: 571;
b. the first nucleic acid sequence has at least 90% sequence identity to SEQ ID NO: 568 or 811, and the second nucleic acid sequence has at least 90% sequence identity to SEQ ID NO: 567;
c. the first nucleic acid sequence has at least 90% sequence identity to SEQ ID NO: 580 or 814, and the second nucleic acid sequence has at least 90% sequence identity to SEQ ID NO: 581 or 815;
d. the first nucleic acid sequence has at least 90% sequence identity to SEQ ID NO: 582 or 816, and the second nucleic acid sequence has at least 90% sequence identity to SEQ ID NO: 583 or 817; and
e. the first nucleic acid sequence has at least 90% sequence identity to SEQ ID NO: 584 or 818, and the second nucleic acid sequence has at least 90% sequence identity to SEQ ID NO: 585 or 819.

In some embodiments, the RNAi agent comprises a sense strand comprising a first nucleic acid sequence, and an antisense strand comprises a second nucleic acid sequence, wherein the first nucleic acid sequence and the second nucleic acid sequence are selected from the group consisting of:
a. the first nucleic acid sequence has at least 95% sequence identity to SEQ ID NO: 564 or 809, and the second nucleic acid sequence has at least 95% sequence identity to SEQ ID NO: 571;
b. the first nucleic acid sequence has at least 95% sequence identity to SEQ ID NO: 568 or 811, and the second nucleic acid sequence has at least 95% sequence identity to SEQ ID NO: 567;
c. the first nucleic acid sequence has at least 95% sequence identity to SEQ ID NO: 580 or 814, and the second nucleic acid sequence has at least 95% sequence identity to SEQ ID NO: 581 or 815;
d. the first nucleic acid sequence has at least 95% sequence identity to SEQ ID NO: 582 or 816, and the second nucleic acid sequence has at least 95% sequence identity to SEQ ID NO: 583 or 817; and
e. the first nucleic acid sequence has at least 95% sequence identity to SEQ ID NO: 584 or 818, and the second nucleic acid sequence has at least 95% sequence identity to SEQ ID NO: 585 or 819.

In some embodiments, the RNAi agent comprises a sense strand comprising a first nucleic acid sequence, and an antisense strand comprises a second nucleic acid sequence, wherein the first nucleic acid sequence and the second nucleic acid sequence are selected from the group consisting of:
a. the first nucleic acid sequence comprises SEQ ID NO: 564 or 809, and the second nucleic acid sequence comprises SEQ ID NO: 571;
b. the first nucleic acid sequence comprises SEQ ID NO: 568 or 811, and the second nucleic acid sequence comprises SEQ ID NO: 567;
c. the first nucleic acid sequence comprises SEQ ID NO: 580 or 814, and the second nucleic acid sequence comprises SEQ ID NO: 581 or 815;
d. the first nucleic acid sequence comprises SEQ ID NO: 582 or 816, and the second nucleic acid sequence comprises SEQ ID NO: 583 or 817; and
e. the first nucleic acid sequence comprises SEQ ID NO: 584 or 818, and the second nucleic acid sequence comprises SEQ ID NO: 585 or 819.

In some embodiments, the RNAi agent comprises a sense strand comprising a first nucleic acid sequence, and an antisense strand comprises a second nucleic acid sequence, wherein the first nucleic acid sequence and the second nucleic acid sequence are selected from the group consisting of:
a. the first nucleic acid sequence consists of SEQ ID NO: 564 or 809, and the second nucleic acid sequence consists of SEQ ID NO: 571;
b. the first nucleic acid sequence consists of SEQ ID NO: 568 or 811, and the second nucleic acid sequence consists of SEQ ID NO: 567;
c. the first nucleic acid sequence consists of SEQ ID NO: 580 or 814, and the second nucleic acid sequence consists of SEQ ID NO: 581 or 815;
d. the first nucleic acid sequence consists of SEQ ID NO: 582 or 816, and the second nucleic acid sequence consists of SEQ ID NO: 583 or 817; and
e. the first nucleic acid sequence consists of SEQ ID NO: 584 or 818, and the second nucleic acid sequence consists of SEQ ID NO: 585 or 819.

In further embodiments, the 5′ terminal nucleotide of the antisense strand is substituted such that the final sequence contains a vinylphosphonate, a phosphate group, or an OH group. For example, for antisense sequences of SEQ ID NOs: 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 563, 566, 567, 569, 570, 571, 575, 579, 581, 583, 585, or a sequence having at least 90% sequence identity thereto, the 5′ phosphate group is replaced with an OH group.

In other embodiments disclosed herein are RNAi agents having a delivery moiety of Formula I conjugated to R:

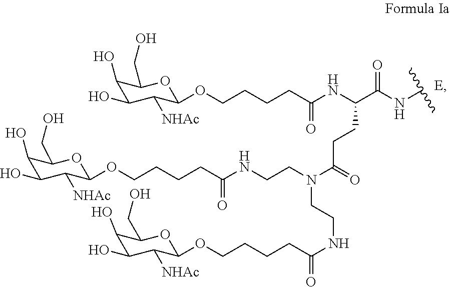

wherein R is a dsRNA comprises a sense strand and an antisense strand, wherein the antisense strand comprises at least 15 contiguous nucleotides that have complementarity to FAS mRNA target sequence of SEQ ID NO:1, and wherein the sense strand and the antisense strand form a region of complementarity of at least 15 nucleotides, and wherein the sense strand and antisense strand are each independently 18 to 23 nucleotides in length, and optionally wherein the sense strand and antisense strand each independently comprise one or more modified nucleotides, and optionally wherein the sense strand and the antisense strand each independently comprise one or more modified internucleotide linkages, and wherein R is optionally conjugated to Formula I via a linker. In further embodiments, the sense or the antisense strand is selected from Table 2, 3A, 3B, 4A, 4B, 7, or 8 disclosed herein. In other embodiments, the antisense or antisense strand of the RNAi agent has a sequence of at least 80%, 81%, 82%, 83%, 84%, 85%, 86%, 87%, 88%, 89%, 90%, 91%, 92%, 93%, 94%, 95%, 96%, 97%, 98%, or 99% sequence identity to the corresponding sequence selected from Table 2, 3A, 3B, 4A, 4B, 7, or 8 herein.

In other embodiments, the RNAi agent disclosed herein comprises a linker. In further embodiments, R is conjugated to Formula I via a linker. In other further embodiments R is conjugated to Formula I via a linker. In further embodiments, the linker comprises a linker of Formula II having connection points A and B or the linker comprises Formula III having connection points C and D, and wherein:

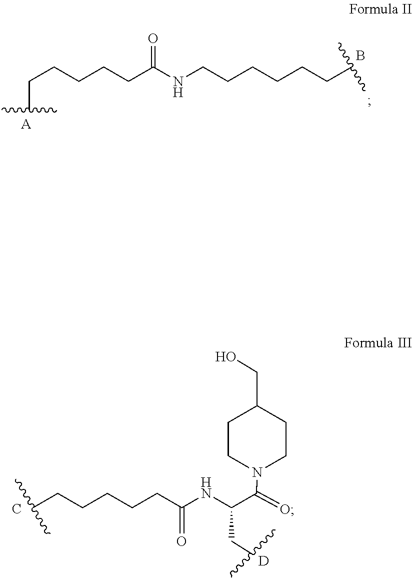

a. Formula I is conjugated to Formula II at connection point A and Formula II is conjugated to a phosphate group at connection point B, and the phosphate group is conjugated to R; or
b. Formula I is conjugated to Formula III at connection point C and Formula III is conjugated to a phosphate group at connection point D, and the phosphate group is further conjugated to R.

In other embodiments wherein the RNAi agent comprises a linker, R is conjugated to Formula I via a linker, and the linker is a linker comprising Formula III having connection points C and D:

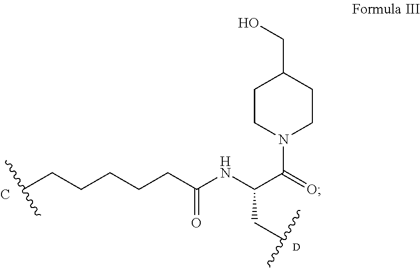

and wherein Formula I is conjugated to Formula III at connection point C and Formula III is conjugated to a phosphate group at connection point D, and the phosphate group is further conjugated to R.

The sense strand and antisense strand of FAS RNAi agent can be synthesized using any nucleic acid polymerization methods known in the art, for example, solid-phase synthesis by employing phosphoramidite chemistry methodology (e.g., Current Protocols in Nucleic Acid Chemistry, Beaucage, S. L. et al. (Edrs.), John Wiley & Sons, Inc., New York, NY, USA), H-phosphonate, phosphortriester chemistry, or enzymatic synthesis. Automated commercial synthesizers can be used, for example, MerMade™ 12 from LGC Biosearch Technologies, or other synthesizers from BioAutomation or Applied Biosystems. Phosphorothioate linkages can be introduced using a sulfurizing reagent such as phenylacetyl disulfide or DDTT (((dimethylaminomethylidene) amino)-3H-1,2,4-dithiazaoline-3-thione). It is well known to use similar techniques and commercially available modified amidites and controlled-pore glass (CPG) products to synthesize modified oligonucleotides.

In still other embodiments, the RNAi agent is capable of decreasing expression of the FAS gene in a liver cell. In other embodiments, the RNAi agents disclosed herein are for use in therapy. In further embodiments, the use is for the treatment of AIH.

The RNAi agents may be formulated into pharmaceutical compositions. Accordingly, disclosed herein are pharmaceutical compositions comprising the RNAi agent disclosed herein, and one or more pharmaceutically acceptable excipients. Pharmaceutical compositions can be prepared by methods well known in the art (e.g., Remington: The Science and Practice of Pharmacy, 23rd edition (2020), A. Loyd et al., Academic Press).

In other embodiment are uses of the RNAi agents herein for the manufacture of a medicament for the treatment of AIH.

In other embodiments are methods of treating AIH, in patients in need thereof, comprising administering a FAS RNAi agent disclosed herein, or a pharmaceutical composition thereof.

The RNAi agent can be administered to the patient intravenously or subcutaneously.

RNAi dosage regimens may be adjusted to provide the optimum desired response (e.g., a therapeutic response). For example, a single bolus may be administered, several divided doses may be administered over time, or the dose may be proportionally reduced or increased as indicated by the exigencies of the therapeutic situation.

Dosage values may vary with the type and severity of the condition to be alleviated. It is further understood that for any particular subject, specific dosage regimens should be adjusted over time according to the individual need and the professional judgment of the person administering or supervising the administration of the compositions.

In other embodiments are methods of decreasing FAS expression in a cell, comprising contacting the cell with an RNAi agent disclosed herein, and incubating the cell for a time sufficient for decreasing the level of FAS mRNA by at least 50% as compared to an untreated or control treated cell.

### Examples

Certain abbreviations are defined as follows: “1,2-DCE” refers to 1,2-dichloroethane; “DCM” refers to dichloromethane; “DIEA” refers to N,N-diisopropylethylamine; “DMF” refers to N,N-dimethylformamide; “DMAP” refers to 4-dimethylaminopyridine; “DMTCl” refers to 4,4′-dimethoxytrityl chloride; “DPP4” refers to dipeptidyl peptidase; “EDC” refers to 1-ethyl-3-(3-dimethylaminopropyl)carbodiimide; “EtOAc” refers to ethyl acetate; “GalNAc” refers to N-acetylgalactosamine; “HATU” refers to 1-[bis(dimethylamino)methylene]-1H-1,2,3-triazolo[4,5-b]pyridinium 3-oxid hexafluorophosphate; “HBTU” refers to 0-(benzotriazol-1-yl)-N,N,N′,N′-tetramethyluronium hexafluorophosphate; “HOBt” refers to 1-hydroxybenzotriazole hydrate; “HPRT” refers to hypoxanthine-guanine phosphoribosyltransferase; “IPA” refers to isopropanol and isopropyl alcohol; “LDHA” refers to lactate dehydrogenase-A; “MeCN” refers to acetonitrile; “MeOH” refers to methanol and methyl alcohol; “MWCO” refers to molecular weight cut-off, “NHS” refers to N-hydroxysuccinimide; “OD” refers to optical density; “PBS” refers to phosphate-buffered saline; “PhSiH3” refers to phenylsilane; “PTS” refers to portable endotoxin testing system; “siRNA” refers to small interfering ribonucleic acid; “TEA” refers to triethylamine; “TFA” refers to trifluoroacetic acid; “THF” refers to tetrahydrofuran; “TLC” refers to thin line chromatography; and “TMP” refers to 2,2,6,6-tetramethylpiperidine.

A delivery moiety comprising Formula I may be made by the following nonlimiting synthetic steps and schemes.

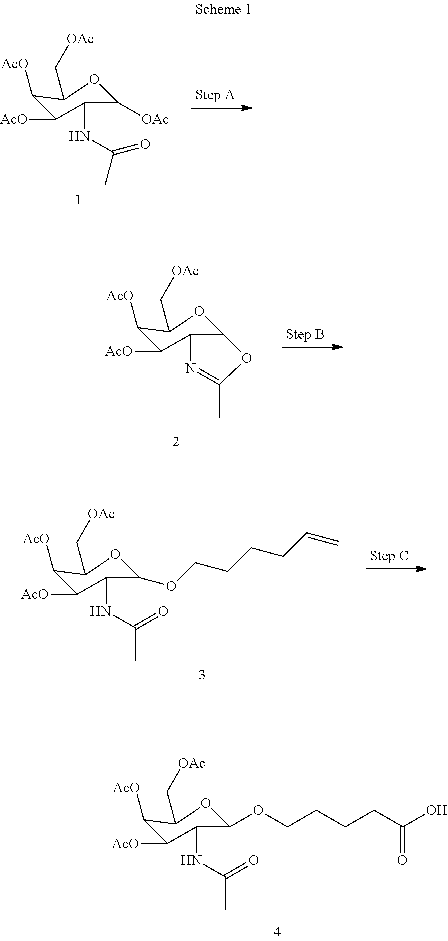 [SMILES: CC(=O)NC1C(OC(C)=O)OC(COC(C)=O)C(OC(C)=O)C1OC(C)=O ; CC(=O)OCC1OC2OC(C)=NC2C(OC(C)=O)C1OC(C)=O ; C=CCCCCOC1OC(COC(C)=O)C(OC(C)=O)C(OC(C)=O)C1NC(C)=O ; CC(=O)NC1C(OCCCCC(=O)O)OC(COC(C)=O)C(OC(C)=O)C1OC(C)=O]

Scheme 1, step A, depicts the cyclization of compound (1) using trimethyl trifluoromethanesulfonate in a solvent such as 1,2-DCE to give compound (2). Step B shows the addition of hex-5-en-1-ol to compound (2) using trimethylsilyl trifluoromethanesulfonate in a solvent such as 1,2-DCE to give compound (3). The oxidation of compound (3) using an appropriate oxidizing agent such as sodium periodate with a catalyst such as ruthenium(III) chloride to give compound (4) is shown in step C.

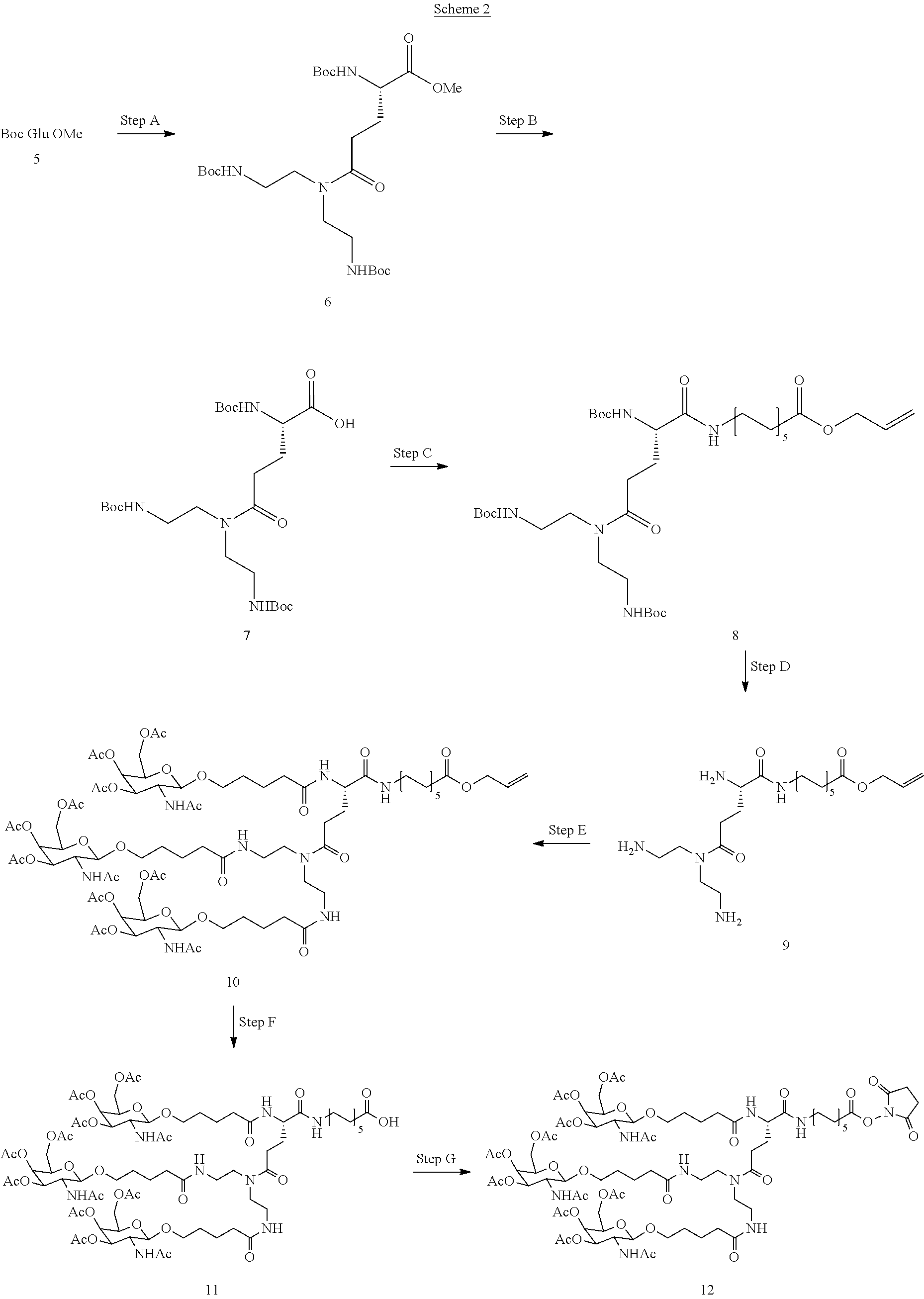

Scheme 2, step A, shows an amide coupling between compound (5) and tert-butyl N-[2-[2-(tert-butoxycarbonylamino)ethylamino]ethyl]carbamate using HBTU and HOBt with an appropriate base such as DIEA in a solvent such as DMF to give compound (6). Step B depicts a basic hydrolysis of compound (6) using a base such as aqueous NaOH in a THF and MeOH solvent system to give compound (7). Step C shows an amide coupling between compound (7) and allyl 11-aminoundecanoate hydrochloride using HATU with an appropriate base such as DIEA in a solvent such as DMF to give compound (8). Step D shows the acidic deprotection of compound (8) with TFA in a solvent such as DCM to give compound (9). The amide coupling between compound (9) and compound (4) using EDC and HOBt in a solvent such as DCM to give compound (10) is shown in step E. Step F shows the deprotection of compound (10) with tetrakis(triphenylphosphine)palladium and PhSiH3 in a solvent such as DCM to give compound (11). Step F depicts the coupling of compound (11) with NHS using EDC in a solvent such as DCM to give compound (12).

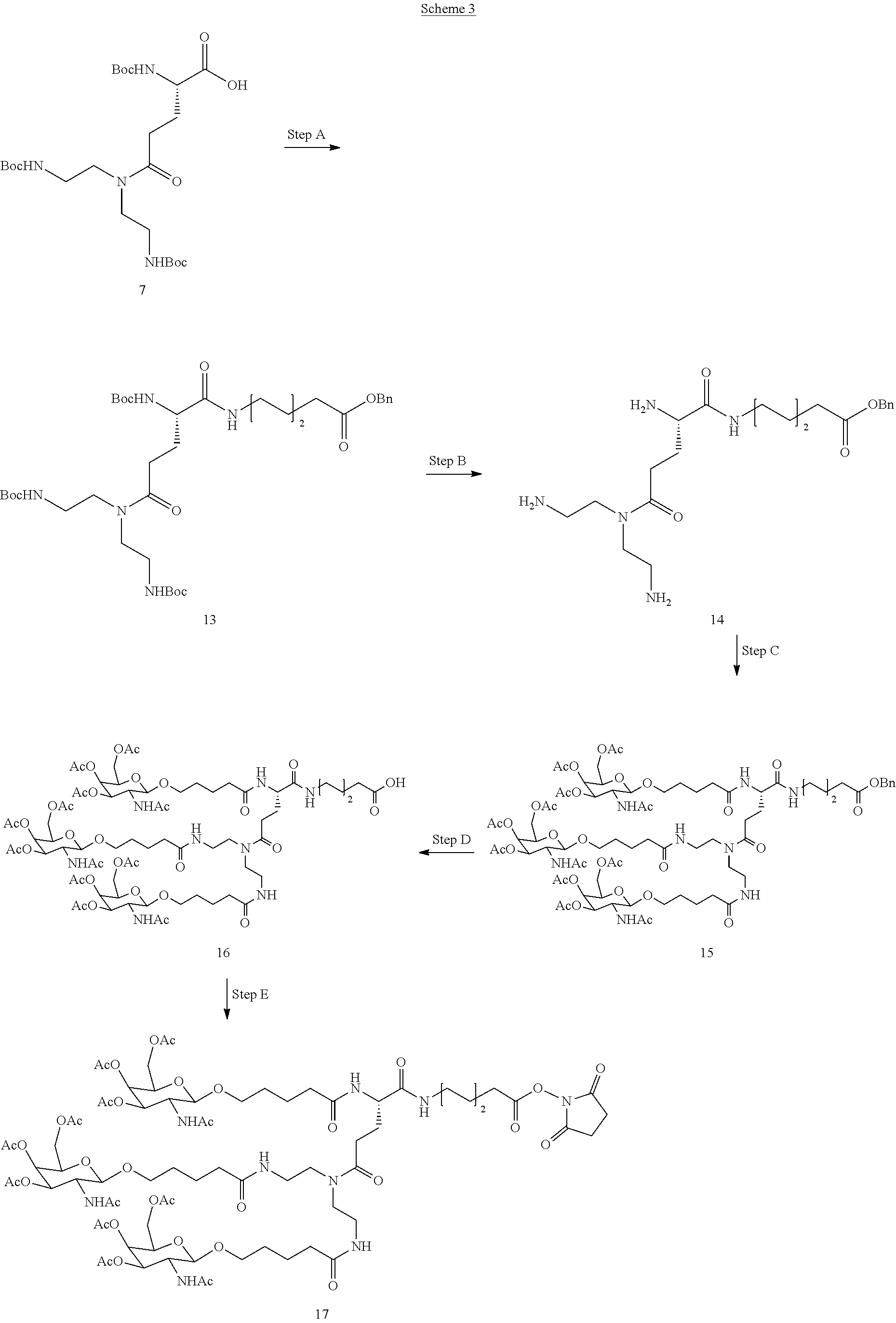

Scheme 3, steps A-C are essentially analogous to those of scheme 2, steps C-E beginning with compound (7) to give compounds (13), (14), and (15). Step D depicts the hydrogenation of compound (15) using palladium on carbon in a solvent such as MeOH to give compound (16). Step E is essentially analogous to the preparation of scheme 2, step G to give compound (17).

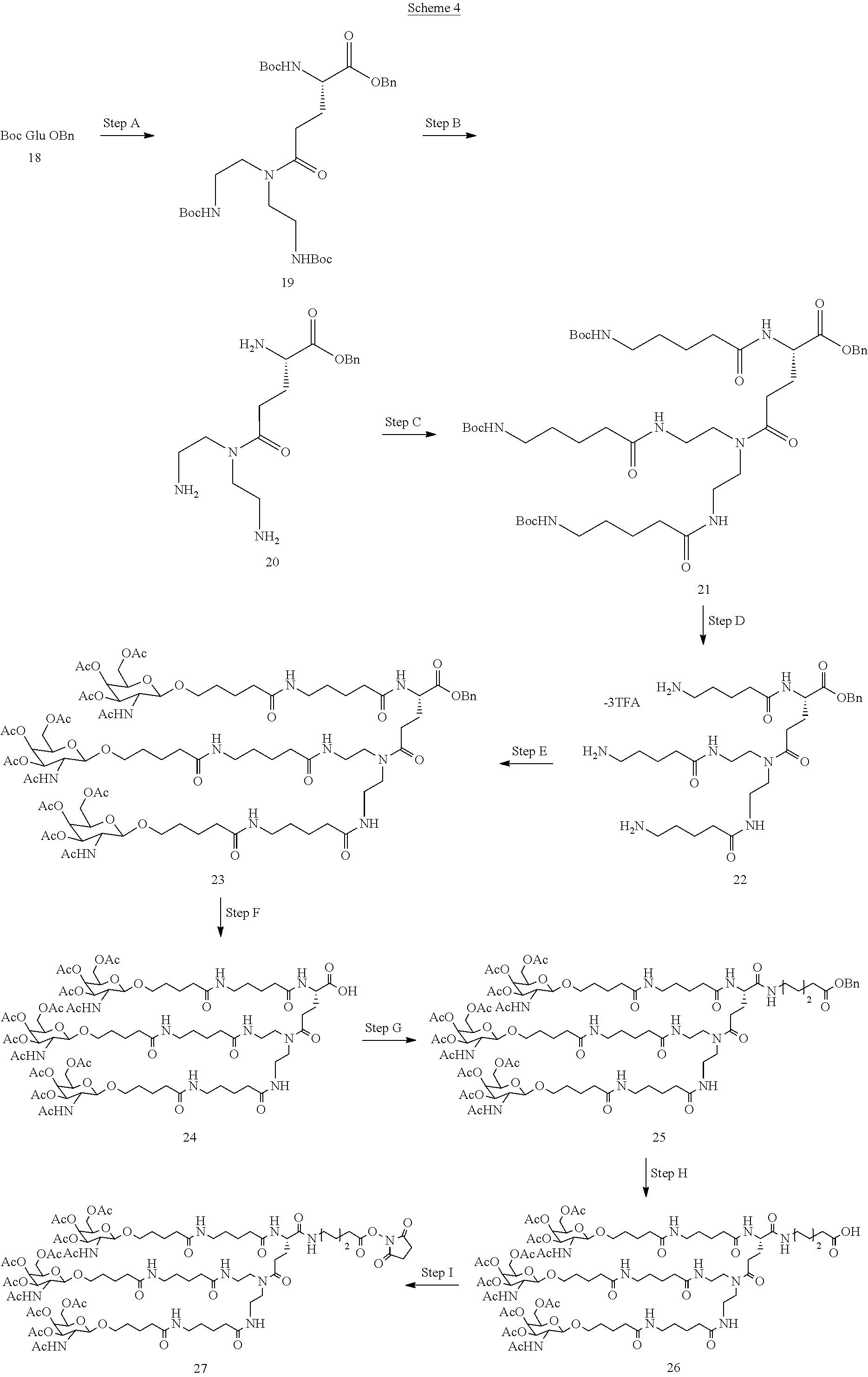

Scheme 4, steps A-I, are composed of a series of amide couplings and deprotections using methods essentially analogous to those found in schemes 2 and 3 beginning with compound (18) to give compound (27).

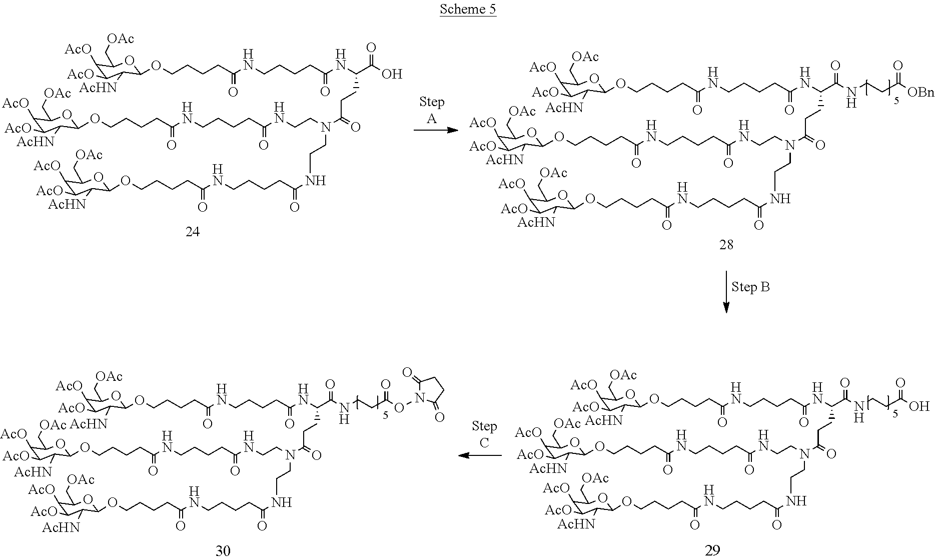

Scheme 5, steps A-C depict methods essentially analogous to those found in scheme 4, steps G-I beginning with compound (24) to give compound (30).

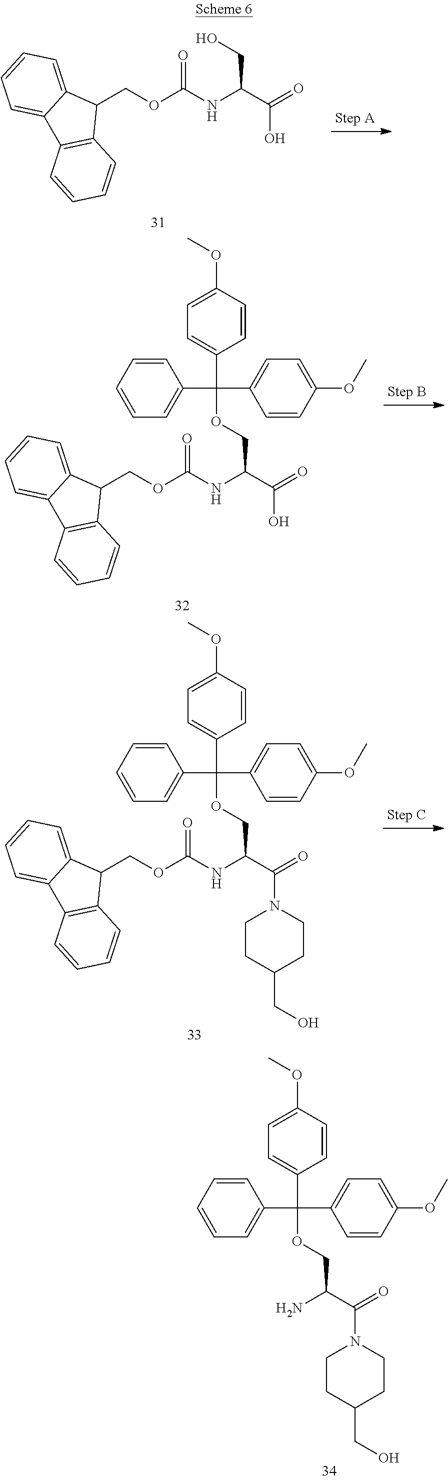 [SMILES: O=C(N[C@@H](CO)C(=O)O)OCC1c2ccccc2-c2ccccc21 ; COc1ccc(C(OC[C@H](NC(=O)OCC2c3ccccc3-c3ccccc32)C(=O)O)(c2ccccc2)c2ccc(OC)cc2)cc1 ; COc1ccc(C(OC[C@H](NC(=O)OCC2c3ccccc3-c3ccccc32)C(=O)N2CCC(CO)CC2)(c2ccccc2)c2ccc(OC)cc2)cc1 ; COc1ccc(C(OC[C@H](N)C(=O)N2CCC(CO)CC2)(c2ccccc2)c2ccc(OC)cc2)cc1]

Scheme 6, step A depicts the protection of compound (31) using DMTCl with a suitable base such as DIEA in a solvent such as DCM to give compound (32). Step B shows an amide coupling between compound (32) and piperidin-4-yl methanol using HBTU and HOBt with TMP in a solvent such as DCM to give compound (33). The deprotection of compound (33) with 20% piperidine in DMF to give compound (34) is shown in step C.

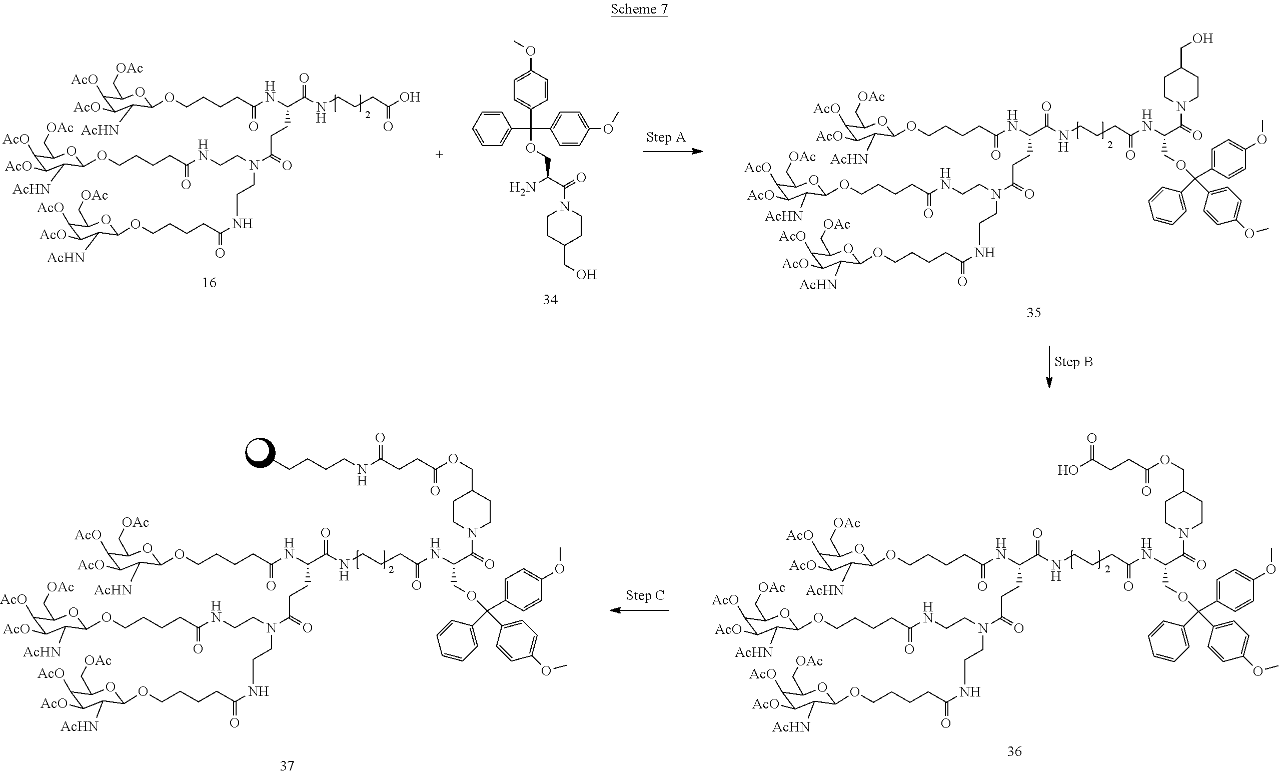

Scheme 7, step A is essentially analogous to scheme 2, step A to give compound (35) from the coupling of compounds (16) and (34). Step B shows the formation of compound (36) by adding succinic anhydride to compound (35) in an appropriate solvent such as DCM with a base system of TEA and DMAP. Step C depicts the loading of compound (36) onto resin with 2-(1H-benzotriazol-1-yl)-1,1,3,3-tetramethyluronium hexafluorophosphate and a base such as DIEA in a solvent system such as MeCN and DCM to give compound (37).

### Preparation 1

### (6,7-Diacetoxy-2-methyl-5,6,7,7a-tetrahydro-3aH-pyrano[3,2-d]oxazol-5-yl)methyl acetate

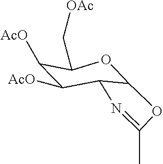 [SMILES: CC(=O)OCC1OC2OC(C)=NC2C(OC(C)=O)C1OC(C)=O]

To a solution of (5-acetamido-3,4,6-triacetoxy-tetrahydropyran-2-yl)methyl acetate (9.00 g, 23.1 mmol) in 1,2-DCE (46 mL) is added trimethylsilyl trifluoromethanesulfonate (6.5 mL, 35 mmol). The mixture is heated to 50° C. and stirred for 18 hours. After this time, the mixture is diluted with DCM (200 mL), washed with saturated NaHCO₃ (200 mL), and saturated aqueous sodium chloride solution (200 mL), dried over sodium sulfate, filtered, and concentrated in vacuo. The resulting residue is purified by silica gel flash chromatography eluting with 0-10% MeOH/DCM to give the title compound (6.434 g, 84%). ES/MS m/z 330 (M+H).

### Preparation 2

### (5-Acetamido-3,4-diacetoxy-6-hex-5-enoxy-tetrahydropyran-2-yl)methyl acetate

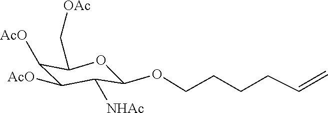 [SMILES: C=CCCCCOC1OC(COC(C)=O)C(OC(C)=O)C(OC(C)=O)C1NC(C)=O]

To a solution of (6,7-diacetoxy-2-methyl-5,6,7,7a-tetrahydro-3aH-pyrano[3,2-d]oxazol-5-yl)methyl acetate (30.43 g, 92.42 mmol) in 1,2-DCE (231 mL) is added hex-5-en-1-ol (22.2 mL, 185 mmol) followed by activated powdered 4 Å molecular sieves (15.6 g). The suspension is stirred at ambient temperature for 30 minutes and trimethylsilyl trifluoromethanesulfonate (19 mL, 101.9 mmol) is then added. The mixture is stirred at ambient temperature for 18 hours. After this time, the solution is filtered through diatomaceous earth and concentrated in vacuo. The resulting residue is purified by silica gel flash chromatography eluting with 30-100% EtOAc/hexanes to give the title compound (34.76 g, 86%). ES/MS m/z 430.4 (M+H).

### Preparation 3

### 5-[3-Acetamido-4,5-diacetoxy-6-(acetoxymethyl)tetrahydropyran-2-yl]oxypentanoic acid

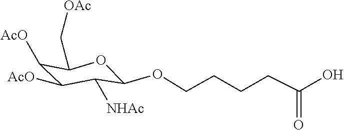 [SMILES: CC(=O)NC1C(OCCCCC(=O)O)OC(COC(C)=O)C(OC(C)=O)C1OC(C)=O]

A solution of (5-acetamido-3,4-diacetoxy-6-hex-5-enoxy-tetrahydropyran-2-yl)methyl acetate (34.76 g, 80.93 mmol) in MeCN (174 mL) and DCM (174 mL) is cooled to 0° C. A solution of sodium periodate (22.4 g, 104.7 mmol) is added and stirring is continued at 0° C. for 10 minutes. After this time, ruthenium(III) chloride (270 mg, 1.3 mmol) is added and the mixture is stirred while warming to ambient temperature. After stirring for 2 hours, additional sodium periodate (66 g, 308.4 mmol) is added and stirring is continued for 18 hours. After this time, the mixture is extracted with 3:1 CH3Cl:IPA (2×500 mL), washed with saturated aqueous sodium chloride solution (1 L), dried over sodium sulfate, filtered, and concentrated in vacuo. The resulting residue is purified by silica gel flash chromatography eluting with 0-40% MeOH/DCM to give the title compound (29.75 g, 82%). ES/MS m/z 448.4 (M+H).

### Preparation 4

### Benzyl 6-aminohexanoate hydrochloride

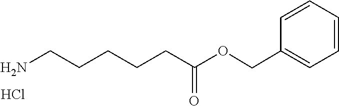 [SMILES: Cl ; NCCCCCC(=O)OCc1ccccc1]

To a suspension of 6-aminohexanoic acid (5.00 g, 38.1 mmol) in THE (38 mL) is added benzyl alcohol (47 mL, 453.7 mmol) and the mixture is cooled to 0° C. Thionyl chloride (8.6 mL, 120 mmol) is added dropwise and the mixture is stirred for 18 hours while warming to ambient temperature. After this time, ether (166 mL) is added and the reaction vessel is transferred to a freezer at −20° C. for 1 hour. After this time, the solid precipitate is collected by filtration to give the title compound (8.57 g, 81%). ES/MS m/z 222 (M+H).

### Preparation 5

### Benzyl 11-aminoundecanoate hydrochloride

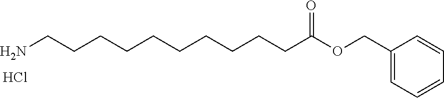 [SMILES: Cl ; NCCCCCCCCCCC(=O)OCc1ccccc1]

The title compound is prepared from 11-aminoundecanoic acid in a manner essentially analogous to the method of preparation 4. ES/MS m/z 292.2 (M+H).

### Preparation 6

### Allyl 11-aminoundecanoate hydrochloride

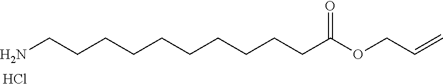 [SMILES: C=CCOC(=O)CCCCCCCCCCN ; Cl]

A vessel is charged with 11-aminoundecanoic acid (9.00 g, 44.7 mmol) in allyl alcohol (42 mL) and the mixture is cooled to 0° C. Thionyl chloride (6.5 mL, 89.4 mmol) is added and the mixture is stirred for 18 hours while warming to ambient temperature. After this time, the mixture is concentrated in vacuo and ether (200 mL) is added to the residue to obtain a white suspension. The mixture is stirred at ambient temperature for 10 minutes and the solid precipitate is collected by filtration to obtain the product (12.0 g, 97%). ES/MS m/z 242.2 (M+H).

### Preparation 7

### (2S)-3-[Bis(4-methoxyphenyl)-phenyl-methoxy]-2-(9H-fluoren-9ylmethoxycarbonylamino) propanoic acid

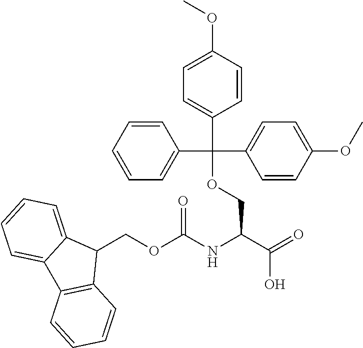 [SMILES: COc1ccc(C(OC[C@H](NC(=O)OCC2c3ccccc3-c3ccccc32)C(=O)O)(c2ccccc2)c2ccc(OC)cc2)cc1]

To a stirring solution of (2S)-2-(9H-fluoren-9-ylmethoxycarbonylamino)-3-hydroxy-propanoic acid (40 g, 0.122 mol) in dry DCM (400 mL) is added DIEA (64 mL, 0.366 mol) at 0° C. under inert atmosphere. To this, a solution of DMTCl (49.6 g, 0.146 mol) in DCM (200 mL) is added slowly. The resulting reaction mixture is brought to ambient temperature and stirred for 16 hours. After this time, the reaction mixture is diluted with water (12.5 vol) and extracted with DCM (25 vol). The organic layer is dried over anhydrous sodium sulphate, filtered, and concentrated in vacuo. The crude obtained is washed with 10% EtOAc/hexane (12.5 vol) and dried under vacuum to give the title compound as a pale brown solid (62 g, crude). This material was taken to next step without any further purification. TLC: 5% MeOH/CH2Cl2 (Rf: 0.5) UV, 254 nM.

### Preparation 8

### 9H-Fluoren-9-ylmethyl N-[(1S)-1-[[bis(4-methoxyphenyl)-phenyl-methoxy]methyl]-2-[4-(hydroxymethyl)-1-piperidyl]-2-oxo-ethyl]carbamate

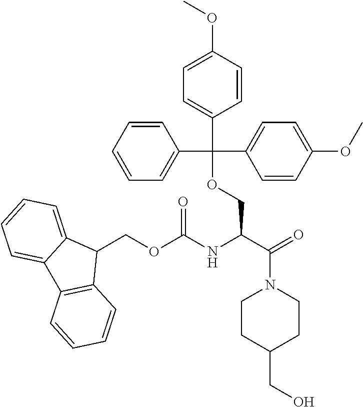 [SMILES: COc1ccc(C(OC[C@H](NC(=O)OCC2c3ccccc3-c3ccccc32)C(=O)N2CCC(CO)CC2)(c2ccccc2)c2ccc(OC)cc2)cc1]

To a stirring solution of (2S)-3-[bis(4-methoxyphenyl)-phenyl-methoxy]-2-(9H-fluoren-9ylmethoxycarbonylamino) propanoic acid (62 g, 0.103 mol) in DCM (750 mL) are added slowly HBTU (78.3 g, 0.206 mol), HOBt (27.9 g, 0.206 mol), and piperidin-4-yl methanol (15.4 g, 0.134 mol) followed by TMP (15 mL, 0.113 mol) at 0° C. under inert atmosphere. The resulting reaction mixture is brought to ambient temperature and stirred for 4 hours. After this time, the reaction mixture is diluted with water (8 vol) and extracted with DCM (15 vol). The organic layer is dried over anhydrous sodium sulphate, filtered, and concentrated in vacuo. The resulting residue is purified by silica gel flash chromatography eluting with 20-40% EtOAc/hexane and 1% MeOH/DCM to give the title compound (40 g, 52% over two steps). 1H NMR (DMSO-d6) δ 7.88 (br d, J=7.5 Hz, 2H), 7.79-7.59 (m, 3H), 7.45-7.12 (m, 13H), 6.92-6.76 (m, 4H), 4.79-4.44 (m, 2H), 4.32 (br d, J=11.4 Hz, 2H), 4.20 (br s, 2H), 3.71 (s, 6H), 3.21 (br s, 4H), 2.99-2.79 (m, 1H), 2.69 (br s, 2H), 1.81-1.43 (m, 3H), 1.08-0.73 (m, 2H).

### Preparation 9

### (2S)-2-Amino-3-[bis(4-methoxyphenyl)-phenyl-methoxy]-1-[4-(hydroxymethyl)-1-piperidyl]propan-1-one

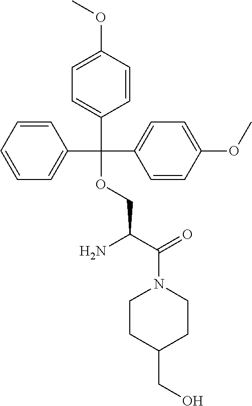 [SMILES: COc1ccc(C(OC[C@H](N)C(=O)N2CCC(CO)CC2)(c2ccccc2)c2ccc(OC)cc2)cc1]

A solution of 20% piperidine in DMF (400 mL) is added slowly to 9H-fluoren-9-ylmethyl N-[(1S)-1-[[bis(4-methoxyphenyl)-phenyl-methoxy]methyl]-2-[4-(hydroxymethyl)-1-piperidyl]-2-oxo-ethyl]carbamate (40 g, 0.055 mol) at 0° C. under inert atmosphere. The resulting reaction mixture is stirred at ambient temperature for 1 hour. After this time, the mixture is diluted with water (15 vol) and extracted with EtOAc (30 vol). The organic layer is dried over anhydrous sodium sulphate, filtered, and concentrated in vacuo. The resulting residue is purified by silica gel flash chromatography eluting with 1-8% MeOH/DCM to give the title compound as an off white solid (13 g, 47%). ES/MS m/z 1009.5 (2M+H).

### Preparation 10

### Methyl (2S)-5-[bis[2-(tert-butoxycarbonylamino)ethyl]amino]-2-(tert-butoxycarbonylamino)-5-oxo-pentanoate

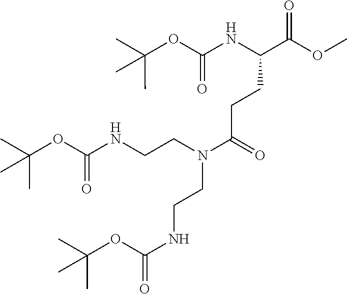 [SMILES: COC(=O)[C@H](CCC(=O)N(CCNC(=O)OC(C)(C)C)CCNC(=O)OC(C)(C)C)NC(=O)OC(C)(C)C]

To a flask containing (S)-4-((tert-butoxycarbonyl)amino)-5-methoxy-5-oxopentanoic acid (7.00 g, 26.8 mmol) and HOBt (4.16 g, 30.8 mmol) are added DMF (179 mL) and (2-(1H-benzotriazol-1-yl)-1,1,3,3-tetramethyluronium hexafluorophosphate (11.7 g, 30.9 mmol). DIEA (14 mL, 80.3 mmol) is added and the mixture is stirred at ambient temperature for 5 minutes. After this time, tert-butyl N-[2-[2-(tert-butoxycarbonylamino)ethylamino]ethyl]carbamate (8.94 g, 29.5 mmol) is added in one portion and stirring is continued at ambient temperature. After stirring for 18 hours, the mixture is diluted with EtOAc (400 mL), washed with water (2×400 mL) and saturated aqueous sodium chloride solution (400 mL), dried over sodium sulfate, filtered, and concentrated in vacuo. The resulting residue is purified by silica gel flash chromatography eluting with 40-100% EtOAc/hexanes to give the title compound (13.01 g, 89%). ES/MS m/z 547.40 (M+H).

### Preparation 11

### (2S)-5-[Bis[2-(tert-butoxycarbonylamino)ethyl]amino]-2-(tert-butoxycarbonylamino)-5-oxo-pentanoic acid

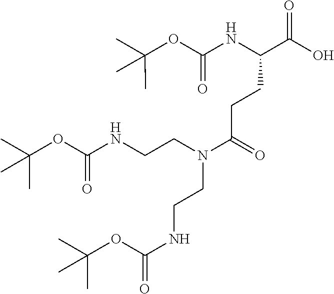 [SMILES: CC(C)(C)OC(=O)NCCN(CCNC(=O)OC(C)(C)C)C(=O)CC[C@H](NC(=O)OC(C)(C)C)C(=O)O]

A flask is charged with methyl (2S)-5-[bis[2-(tert-butoxycarbonylamino)ethyl]amino]-2-(tert-butoxycarbonylamino)-5-oxo-pentanoate (13.01 g, 23.8 mmol), THE (120 mL), and MeOH (120 mL). 1N NaOH (71 mL, 71 mmol) is added and the mixture is stirred at ambient temperature. After 1 hour, the mixture is concentrated in vacuo and redissolved in water (300 mL). 5N HCl (12 mL) is added to bring the pH to 4. The mixture is extracted with DCM (3×300 mL) and the combined organic layers are washed with saturated aqueous sodium chloride solution (1 L), dried over sodium sulfate, filtered, and concentrated to give the title compound (12.41 g, 98%). ES/MS m/z 531.60 (M−H).

### Preparation 12

### Allyl 11-[[(2S)-5-[bis[2-(tert-butoxycarbonylamino)ethyl]amino]-2-(tert-butoxycarbonylamino)-5-oxo-pentanoyl]amino]undecanoate

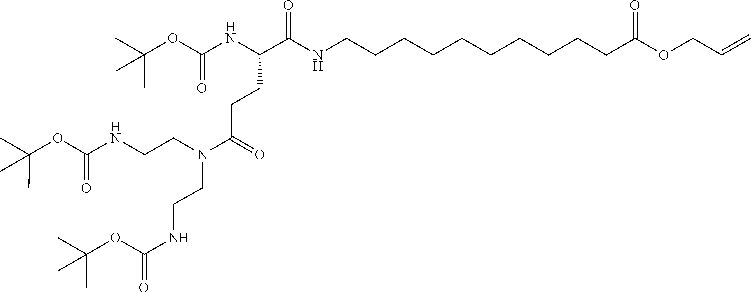 [SMILES: C=CCOC(=O)CCCCCCCCCCNC(=O)[C@H](CCC(=O)N(CCNC(=O)OC(C)(C)C)CCNC(=O)OC(C)(C)C)NC(=O)OC(C)(C)C]

To a flask containing (2S)-5-[bis[2-(tert-butoxycarbonylamino)ethyl]amino]-2-(tert-butoxycarbonylamino)-5-oxo-pentanoic acid (500 mg, 0.94 mmol) and allyl 11-aminoundecanoate hydrochloride (313 mg, 1.13 mmol) is added DMF (6.25 mL) and (1-[bis(dimethylamino)methylene]-1H-1,2,3-triazolo[4,5-b]pyridinium 3-oxide hexafluorophosphate (428 mg, 1.12 mmol). Following addition of DIEA (0.5 mL, 3 mmol) the mixture is stirred at ambient temperature for 18 hours. After this time, the mixture is diluted with EtOAc (200 mL), washed with water (3×200 mL) and saturated aqueous sodium chloride solution (200 mL), dried over sodium sulfate, filtered, and concentrated in vacuo. The resulting residue is purified by silica gel flash chromatography eluting with 40-100% EtOAc/hexanes to give the title compound (687 mg, 97%). 1H NMR (DMSO-d6) δ 7.78-7.64 (m, 1H), 6.98-6.7 (m, 2H), 5.96-5.84 (m, 1H), 5.31-5.25 (m, 1H), 5.23-5.17 (m, 1H), 4.56-4.50 (m, 2H), 3.88-3.67 (m, 1H), 3.30-3.19 (m, 4H), 3.11-2.91 (m, 6H), 2.35-2.12 (m, 4H), 1.88-1.65 (m, 2H), 1.58-1.47 (m, 2H), 1.46-1.30 (m, 30H), 1.30-1.18 (m, 12H).

### Preparation 13

### Allyl (S)-11-(2-amino-5-(bis(2-aminoethyl)amino)-5-oxopentanamido)undecanoate

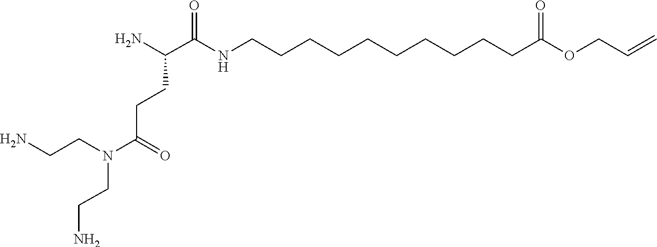 [SMILES: C=CCOC(=O)CCCCCCCCCCNC(=O)[C@@H](N)CCC(=O)N(CCN)CCN]

To a solution of allyl 11-[[(2S)-5-[bis[2-(tert-butoxycarbonylamino)ethyl]amino]-2-(tert-butoxycarbonylamino)-5-oxo-pentanoyl]amino]undecanoate (687 mg, 0.91 mmol) in DCM (15 mL) is added TFA (15 mL). The mixture is stirred at ambient temperature. After 1.5 hours, the mixture is concentrated in vacuo. The residue is taken up in MeOH and applied to an ion exchange cartridge. The cartridge is eluted with MeOH (150 mL) followed by 7N NH3/MeOH (150 mL). The basic fraction is concentrated in vacuo to give the title compound (410 mg, 99%). ES/MS m/z 456.4 (M+H).

### Preparation 14

### Allyl 11-[[(2S)-2-[5-[3-acetamido-4,5-diacetoxy-6-(acetoxymethyl)tetrahydropyran-2-yl]oxypentanoylamino]-5-[bis[2-[5-[3-acetamido-4,5-diacetoxy-6-(acetoxymethyl)tetrahydropyran-2-yl]oxypentanoylamino]ethyl]amino]-5-oxo-pentanoyl]amino]undecanoate

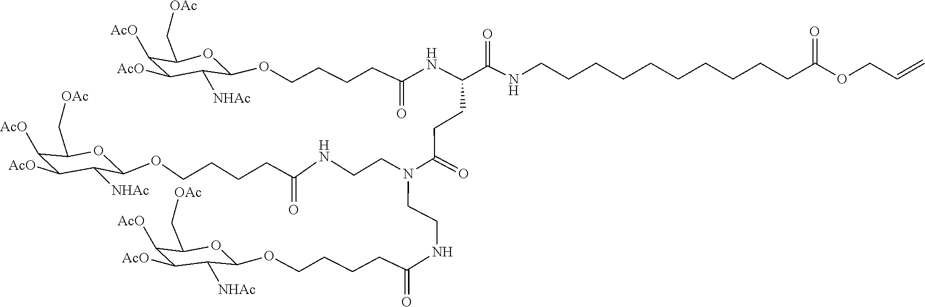

A flask is charged with 5-[3-acetamido-4,5-diacetoxy-6-(acetoxymethyl)tetrahydropyran-2-yl]oxypentanoic acid (489 mg, 1.09 mmol) and allyl (S)-11-(2-amino-5-(bis(2-aminoethyl)amino)-5-oxopentanamido)undecanoate (150 mg, 0.33 mmol). DCM (3.35 mL) is added followed by 1-hydroxybenzotriazole monohydrate (164 mg, 1.07 mmol) and 1-(3-dimethylaminopropyl)-3-ethylcarbodiimide hydrochloride (206 mg, 1.07 mmol). The mixture is stirred at ambient temperature for 18 hours. After this time, the solution is diluted with EtOAc (100 mL), washed with saturated NaHCO₃ (2×100 mL), saturated aqueous NH4Cl (100 mL), and saturated aqueous sodium chloride solution (100 mL). The organic layer is dried over sodium sulfate, filtered, and concentrated in vacuo. The resulting residue is purified by silica gel flash chromatography eluting with 0-10% MeOH/DCM to give the title compound (424 mg, 74%). ES/MS m/z 872.80 (M+2H)/2.

### Preparation 15

### 11-[[(2S)-2-[5-[3-Acetamido-4,5-diacetoxy-6-(acetoxymethyl)tetrahydropyran-2-yl]oxypentanoylamino]-5-[bis[2-[5-[3-acetamido-4,5-diacetoxy-6-(acetoxymethyl)tetrahydropyran-2-yl]oxypentanoylamino]ethyl]amino]-5-oxo-pentanoyl]amino]undecanoic acid

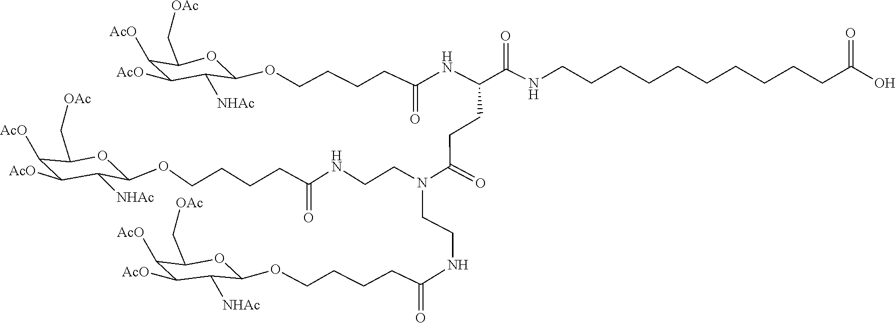

To a solution of allyl 11-[[(2S)-2-[5-[3-acetamido-4,5-diacetoxy-6-(acetoxymethyl)tetrahydropyran-2-yl]oxypentanoylamino]-5-[bis[2-[5-[3-acetamido-4,5-diacetoxy-6-(acetoxymethyl)tetrahydropyran-2-yl]oxypentanoylamino]ethyl]amino]-5-oxo-pentanoyl]amino]undecanoate (354 mg, 0.20 mmol) in DCM (2 mL) is added tetrakis(triphenylphosphine)palladium (29 mg, 0.02 mmol) followed by PhSiH3 (51 uL, 0.41 mmol). The mixture is stirred at ambient temperature for 2 hours, after which it is diluted with saturated aqueous NaHCO₃ (100 mL). 1N NaOH (15 mL) is added to bring the pH to about 10. The aqueous solution is washed with DCM (3×100 mL) and then acidified with concentrated HCl (5 mL) and then aqueous 5N HCl (15 mL). The aqueous layer is extracted with DCM (100 mL) and the organic layer is dried over sodium sulfate, filtered, and concentrated in vacuo. The resulting residue is purified by silica gel flash chromatography eluting with 0-20% MeOH/DCM to give the title compound (151 mg, 44%). ES/MS m/z 852.60 (M+2H)/2.

### Preparation 16

### (2,5-Dioxopyrrolidin-1-yl) 11-[[(2S)-2-[5-[3-acetamido-4,5-diacetoxy-6-(acetoxymethyl)tetrahydropyran-2-yl]oxypentanoylamino]-5-[bis[2-[5-[3-acetamido-4,5-diacetoxy-6-(acetoxymethyl)tetrahydropyran-2-yl]oxypentanoylamino]ethyl]amino]-5-oxo-pentanoyl]amino]undecanoate

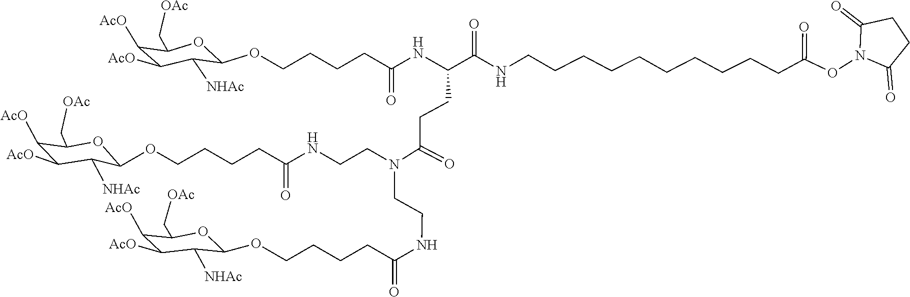 [SMILES: CC(=O)NC1C(OCCCCC(=O)NCCN(CCNC(=O)CCCCOC2OC(COC(C)=O)C(OC(C)=O)C(OC(C)=O)C2NC(C)=O)C(=O)CC[C@H](NC(=O)CCCCOC2OC(COC(C)=O)C(OC(C)=O)C(OC(C)=O)C2NC(C)=O)C(=O)NCCCCCCCCCCC(=O)ON2C(=O)CCC2=O)OC(COC(C)=O)C(OC(C)=O)C1OC(C)=O]

To a reaction vial are added 11-[[(2S)-2-[5-[3-acetamido-4,5-diacetoxy-6-(acetoxymethyl)tetrahydropyran-2-yl]oxypentanoylamino]-5-[bis[2-[5-[3-acetamido-4,5-diacetoxy-6-(acetoxymethyl)tetrahydropyran-2-yl]oxypentanoylamino]ethyl]amino]-5-oxo-pentanoyl]amino]undecanoic acid (50 mg, 0.03 mmol), N-hydroxysuccinimide (5 mg, 0.04 mmol), and 1-(3-dimethylaminopropyl)-3-ethylcarbodiimide hydrochloride (8 mg, 0.04 mmol). DCM (0.3 mL) is added and the mixture is stirred at ambient temperature. After 18 hours, the mixture is loaded directly onto a silica gel cartridge and the crude mixture is purified by silica gel flash chromatography eluting with 0-10% MeOH/DCM to give the title compound (49 mg, 93%). ES/MS m/z 901.40 (M+2H)/2.

### Preparation 17

### Benzyl 6-[[(2S)-5-[bis[2-(tert-butoxycarbonylamino)ethyl]amino]-2-(tert-butoxycarbonylamino)-5-oxo-pentanoyl]amino]hexanoate

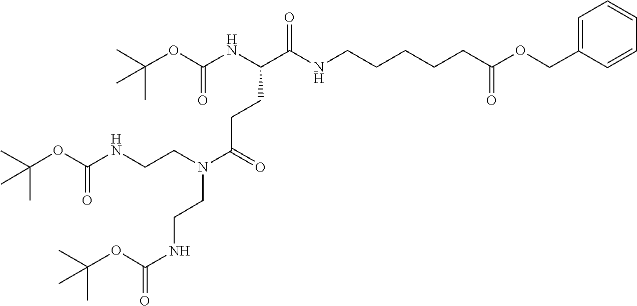 [SMILES: CC(C)(C)OC(=O)NCCN(CCNC(=O)OC(C)(C)C)C(=O)CC[C@H](NC(=O)OC(C)(C)C)C(=O)NCCCCCC(=O)OCc1ccccc1]

The title compound is prepared from (2S)-5-[bis[2-(tert-butoxycarbonylamino)ethyl]amino]-2-(tert-butoxycarbonylamino)-5-oxo-pentanoic acid and benzyl 6-aminohexanoate hydrochloride in a manner essentially analogous to the method of preparation 10. ES/MS m/z 736.40 (M+H).

### Preparation 18

### Benzyl 6-[[(2S)-2-amino-5-[bis(2-aminoethyl)amino]-5-oxo-pentanoyl]amino]hexanoate tris(trifluoroacetic acid)

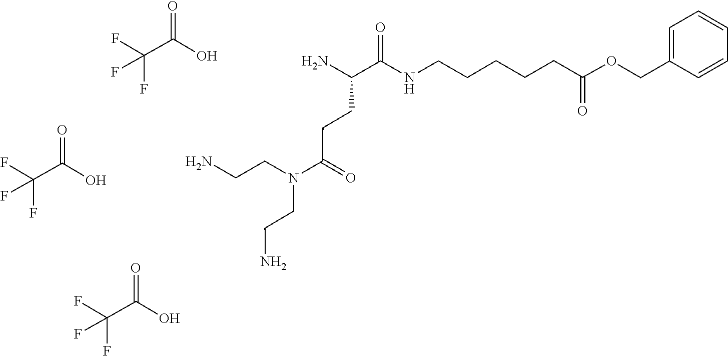 [SMILES: O=C(O)C(F)(F)F ; O=C(O)C(F)(F)F ; NCCN(CCN)C(=O)CC[C@H](N)C(=O)NCCCCCC(=O)OCc1ccccc1 ; O=C(O)C(F)(F)F]

To a solution of benzyl 6-[[(2S)-5-[bis[2-(tert-butoxycarbonylamino)ethyl]amino]-2-(tert-butoxycarbonylamino)-5-oxo-pentanoyl]amino]hexanoate (15.47 g, 21.02 mmol) in DCM (105 mL) is added TFA (16 mL, 210.2 mmol). The mixture is stirred at ambient temperature for 24 hours. After this time, additional TFA (16 mL, 210.2 mmol) is added and stirring is continued for an additional 2 hours. After this time, the mixture is concentrated in vacuo. The resulting residue is azeotroped with toluene (2×30 mL). The resulting oil is further dried in a vacuum oven at 40° C. for 4 hours to give the title compound (28.08 g, 58% purity accounting for residual toluene, 99+%). ES/MS m/z 436.40 (M+H). The compound is dissolved in 70 mL DMF to make a 0.3M solution that is used in the next step.

### Preparation 19

### Benzyl 6-[[(2S)-2-[5-[3-acetamido-4,5-diacetoxy-6-(acetoxymethyl)tetrahydropyran-2-yl]oxypentanoylamino]-5-[bis[2-[5-[3-acetamido-4,5-diacetoxy-6-(acetoxymethyl)tetrahydropyran-2-yl]oxypentanoylamino]ethyl]amino]-5-oxo-pentanoyl]amino]hexanoate

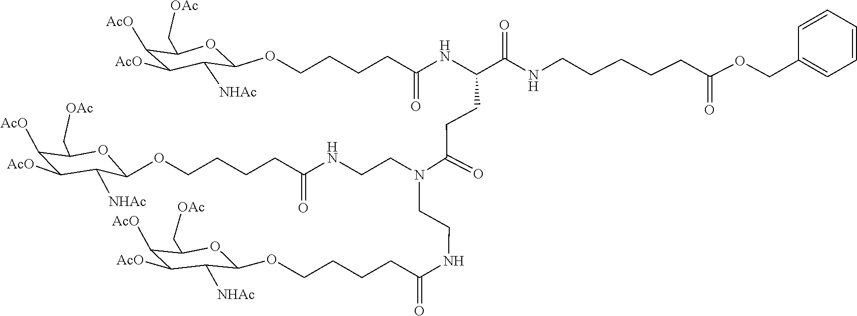 [SMILES: CC(=O)NC1C(OCCCCC(=O)NCCN(CCNC(=O)CCCCOC2OC(COC(C)=O)C(OC(C)=O)C(OC(C)=O)C2NC(C)=O)C(=O)CC[C@H](NC(=O)CCCCOC2OC(COC(C)=O)C(OC(C)=O)C(OC(C)=O)C2NC(C)=O)C(=O)NCCCCCC(=O)OCc2ccccc2)OC(COC(C)=O)C(OC(C)=O)C1OC(C)=O]

The title compound is prepared from 5-[3-acetamido-4,5-diacetoxy-6-(acetoxymethyl)tetrahydropyran-2-yl]oxypentanoic acid and benzyl 6-[[(2S)-2-amino-5-[bis(2-aminoethyl)amino]-5-oxo-pentanoyl]amino]hexanoate tris trifluoroacetic acid and in a manner essentially analogous to the method of preparation 10. ES/MS m/z 862 (M+2H)/2.

### Preparation 20

### 6-[[(2S)-2-[5-[3-Acetamido-4,5-diacetoxy-6-(acetoxymethyl)tetrahydropyran-2-yl]oxypentanoylamino]-5-[bis[2-[5-[3-acetamido-4,5-diacetoxy-6-(acetoxymethyl)tetrahydropyran-2-yl]oxypentanoylamino]ethyl]amino]-5-oxo-pentanoyl]amino]hexanoic acid

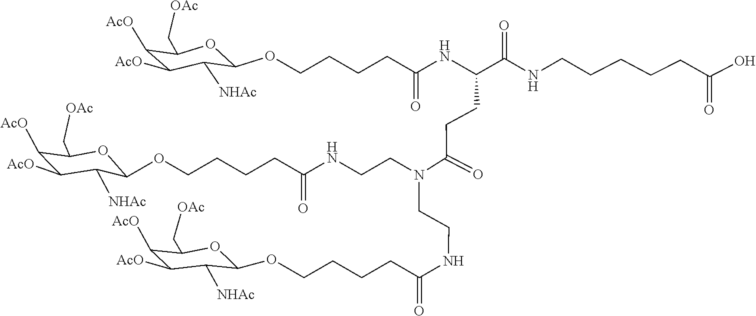 [SMILES: CC(=O)NC1C(OCCCCC(=O)NCCN(CCNC(=O)CCCCOC2OC(COC(C)=O)C(OC(C)=O)C(OC(C)=O)C2NC(C)=O)C(=O)CC[C@H](NC(=O)CCCCOC2OC(COC(C)=O)C(OC(C)=O)C(OC(C)=O)C2NC(C)=O)C(=O)NCCCCCC(=O)O)OC(COC(C)=O)C(OC(C)=O)C1OC(C)=O]

Palladium on carbon (1.90 g, 0.89 mmol, 5 mass %, 50% wet) is placed in a round-bottom flask and the vessel is evacuated and backfilled with nitrogen three times. A solution of benzyl 6-[[(2S)-2-[5-[3-acetamido-4,5-diacetoxy-6-(acetoxymethyl)tetrahydropyran-2-yl]oxypentanoylamino]-5-[bis[2-[5-[3-acetamido-4,5-diacetoxy-6-(acetoxymethyl)tetrahydropyran-2-yl]oxypentanoylamino]ethyl]amino]-5-oxo-pentanoyl]amino]hexanoate (15.41 g, 8.94 mmol) in MeOH (178 mL) is added via syringe. The flask is evacuated and backfilled with 1 atm hydrogen and the mixture is stirred at ambient temperature under 1 atm hydrogen for 18 hours. After this time, the mixture is filtered through diatomaceous earth and the filtrate is concentrated in vacuo to give the title compound (13.85 g, 95%). ES/MS m/z 817.2 (M+2H)/2.

### Preparation 21

### (2,5-Dioxopyrrolidin-1-yl) 6-[[(2S)-2-[5-[3-acetamido-4,5-diacetoxy-6-(acetoxymethyl)tetrahydropyran-2-yl]oxypentanoylamino]-5-[bis[2-[5-[3-acetamido-4,5-diacetoxy-6-(acetoxymethyl)tetrahydropyran-2-yl]oxypentanoylamino]ethyl]amino]-5-oxo-pentanoyl]amino]hexanoate

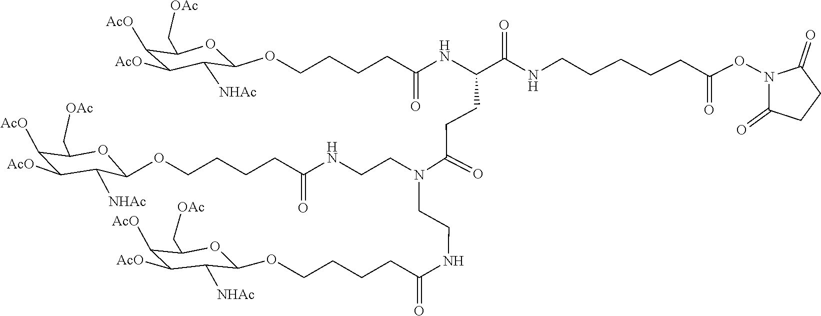 [SMILES: CC(=O)NC1C(OCCCCC(=O)NCCN(CCNC(=O)CCCCOC2OC(COC(C)=O)C(OC(C)=O)C(OC(C)=O)C2NC(C)=O)C(=O)CC[C@H](NC(=O)CCCCOC2OC(COC(C)=O)C(OC(C)=O)C(OC(C)=O)C2NC(C)=O)C(=O)NCCCCCC(=O)ON2C(=O)CCC2=O)OC(COC(C)=O)C(OC(C)=O)C1OC(C)=O]

The title compound is prepared from 6-[[(2S)-2-[5-[3-acetamido-4,5-diacetoxy-6-(acetoxymethyl)tetrahydropyran-2-yl]oxypentanoylamino]-5-[bis[2-[5-[3-acetamido-4,5-diacetoxy-6-(acetoxymethyl)tetrahydropyran-2-yl]oxypentanoylamino]ethyl]amino]-5-oxo-pentanoyl]amino]hexanoic acid in a manner essentially analogous to the method of preparation 16. ES/MS m/z 866.20 (M+2H)/2.

### Preparation 22

### Benzyl (2S)-5-[bis[2-(tert-butoxycarbonylamino)ethyl]amino]-2-(tert-butoxycarbonylamino)-5-oxo-pentanoate

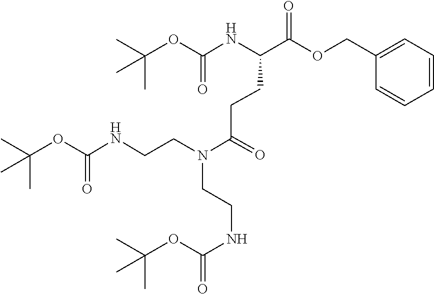 [SMILES: CC(C)(C)OC(=O)NCCN(CCNC(=O)OC(C)(C)C)C(=O)CC[C@H](NC(=O)OC(C)(C)C)C(=O)OCc1ccccc1]

The title compound is prepared from tert-butyl N-[2-[2-(tert-butoxycarbonylamino)ethylamino]ethyl]carbamate and (4S)-5-benzyloxy-4-(tert-butoxycarbonylamino)-5-oxo-pentanoic acid in a manner essentially analogous to the method of preparation 12. ES/MS m/z 623.6 (M+H).

### Preparation 23

### Benzyl (2S)-2-amino-5-[bis(2-aminoethyl)amino]-5-oxo-pentanoate tris(trifluoroacetic acid) salt

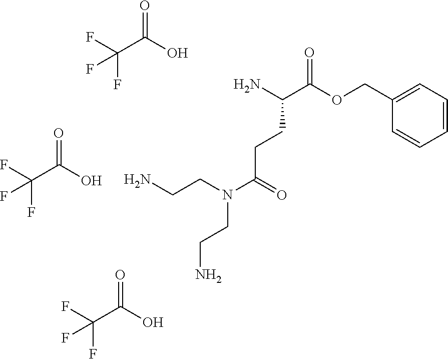 [SMILES: O=C(O)C(F)(F)F ; O=C(O)C(F)(F)F ; NCCN(CCN)C(=O)CC[C@H](N)C(=O)OCc1ccccc1 ; O=C(O)C(F)(F)F]

The title compound is prepared from benzyl (2S)-5-[bis[2-(tert-butoxycarbonylamino)ethyl]amino]-2-(tert-butoxycarbonylamino)-5-oxo-pentanoate in a manner essentially analogous to the method of preparation 18. ES/MS m/z 323.2 (M+H).

### Preparation 24

### Benzyl (2S)-5-[bis[2-[5-(tert-butoxycarbonylamino)pentanoylamino]ethyl]amino]-2-[5-(tert-butoxycarbonylamino)pentanoylamino]-5-oxo-pentanoate

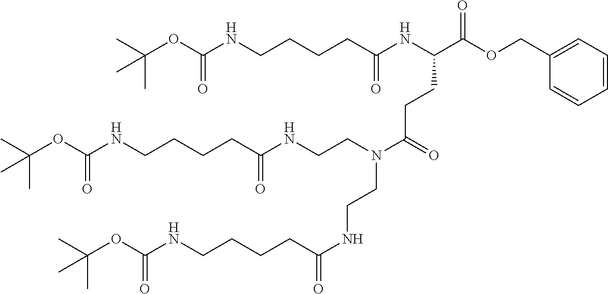 [SMILES: CC(C)(C)OC(=O)NCCCCC(=O)NCCN(CCNC(=O)CCCCNC(=O)OC(C)(C)C)C(=O)CC[C@H](NC(=O)CCCCNC(=O)OC(C)(C)C)C(=O)OCc1ccccc1]

The title compound is prepared from 5-(tert-butoxycarbonylamino)pentanoic acid and benzyl (2S)-2-amino-5-[bis(2-aminoethyl)amino]-5-oxo-pentanoate tris(trifluoroacetic acid) salt in a manner essentially analogous to the method of preparation 10. ES/MS m/z 920.6 (M+H).

### Preparation 25

### Benzyl (2S)-2-(5-aminopentanoylamino)-5-[bis[2-(5-aminopentanoylamino)ethyl]amino]-5-oxo-pentanoate tris(trifluoroacetic acid) salt

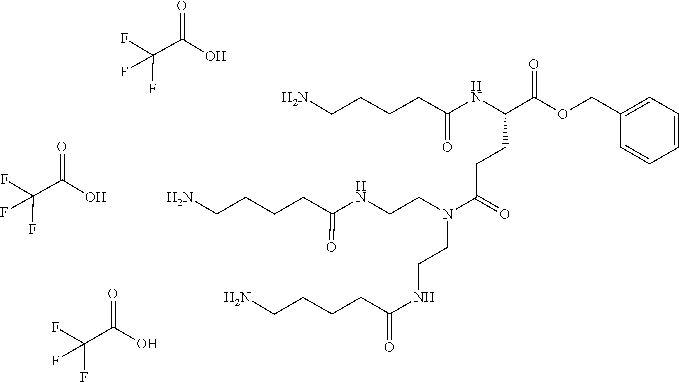 [SMILES: O=C(O)C(F)(F)F ; O=C(O)C(F)(F)F ; O=C(O)C(F)(F)F ; NCCCCC(=O)NCCN(CCNC(=O)CCCCN)C(=O)CC[C@H](NC(=O)CCCCN)C(=O)OCc1ccccc1]

The title compound is prepared from benzyl (2S)-5-[bis[2-[5-(tert-butoxycarbonylamino)pentanoylamino]ethyl]amino]-2-[5-(tert-butoxycarbonylamino)pentanoylamino]-5-oxo-pentanoate in a manner essentially analogous to the method of preparation 18. ES/MS m/z 620.4 (M+H).

### Preparation 26

### Benzyl (2S)-2-[5-[5-[3-acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoate

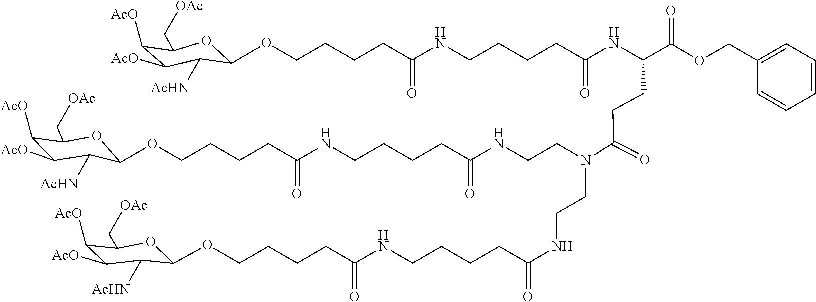 [SMILES: CC(=O)NC1C(OCCCCC(=O)NCCCCC(=O)NCCN(CCNC(=O)CCCCNC(=O)CCCCOC2OC(COC(C)=O)C(OC(C)=O)C(OC(C)=O)C2NC(C)=O)C(=O)CC[C@H](NC(=O)CCCCNC(=O)CCCCOC2OC(COC(C)=O)C(OC(C)=O)C(OC(C)=O)C2NC(C)=O)C(=O)OCc2ccccc2)OC(COC(C)=O)C(OC(C)=O)C1OC(C)=O]

The title compound is prepared from 5-[3-acetamido-4,5-diacetoxy-6-(acetoxymethyl)tetrahydropyran-2-yl]oxypentanoic acid and benzyl (2S)-2-(5-aminopentanoylamino)-5-[bis[2-(5-aminopentanoylamino)ethyl]amino]-5-oxo-pentanoate tris(trifluoroacetic acid) salt and in a manner essentially analogous to the method of preparation 10. ES/MS m/z 954.80 (M+2H)/2.

### Preparation 27

### (2S)-2-[5-[5-[3-Acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoic acid

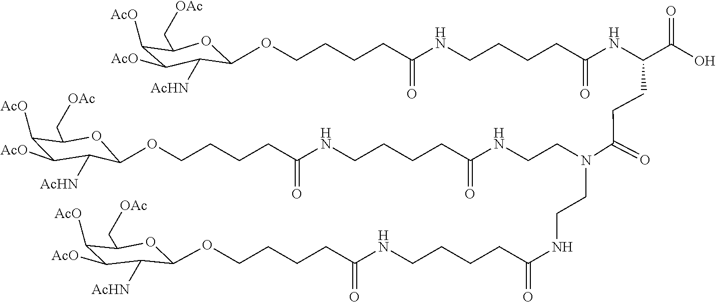 [SMILES: CC(=O)NC1C(OCCCCC(=O)NCCCCC(=O)NCCN(CCNC(=O)CCCCNC(=O)CCCCOC2OC(COC(C)=O)C(OC(C)=O)C(OC(C)=O)C2NC(C)=O)C(=O)CC[C@H](NC(=O)CCCCNC(=O)CCCCOC2OC(COC(C)=O)C(OC(C)=O)C(OC(C)=O)C2NC(C)=O)C(=O)O)OC(COC(C)=O)C(OC(C)=O)C1OC(C)=O]

A round-bottom flask is charged with palladium on carbon (467 mg, 0.22 mmol, 5 mass %, 50% wet) and the flask is evacuated and backfilled with nitrogen three times. A solution of benzyl (2S)-2-[5-[5-[3-acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoate (4.19 g, 2.20 mmol) in MeOH (44 mL) is added via syringe followed by three drops of acetic acid. The flask is evacuated and backfilled with 1 atm hydrogen and the mixture is stirred at ambient temperature under 1 atm hydrogen. After 2 hours, the mixture is filtered through diatomaceous earth and the filtrate is concentrated in vacuo to give the title compound (3.99 g, 99+%). ES/MS m/z 909.6 (M+2H)/2.

### Preparation 28

### Benzyl 6-[[(2S)-2-[5-[5-[3-acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoyl]amino]hexanoate

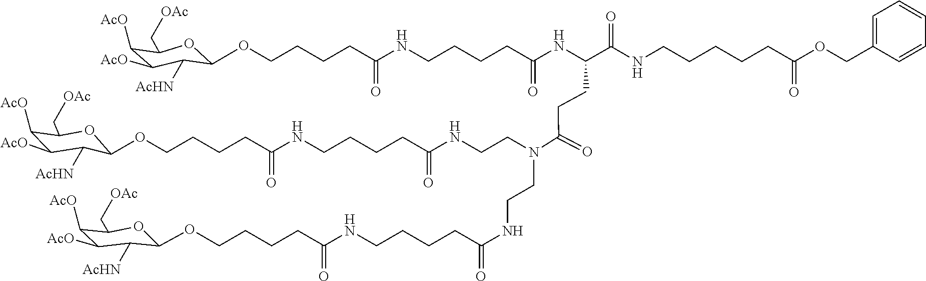 [SMILES: CC(=O)NC1C(OCCCCC(=O)NCCCCC(=O)NCCN(CCNC(=O)CCCCNC(=O)CCCCOC2OC(COC(C)=O)C(OC(C)=O)C(OC(C)=O)C2NC(C)=O)C(=O)CC[C@H](NC(=O)CCCCNC(=O)CCCCOC2OC(COC(C)=O)C(OC(C)=O)C(OC(C)=O)C2NC(C)=O)C(=O)NCCCCCC(=O)OCc2ccccc2)OC(COC(C)=O)C(OC(C)=O)C1OC(C)=O]

The title compound is prepared from (2S)-2-[5-[5-[3-acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoic acid and benzyl 6-aminohexanoate hydrochloride and in a manner essentially analogous to the method of preparation 10. ES/MS m/z 1011.6 (M+2H)/2.

### Preparation 29

### 6-[[(2S)-2-[5-[5-[3-Acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoyl]amino]hexanoic acid

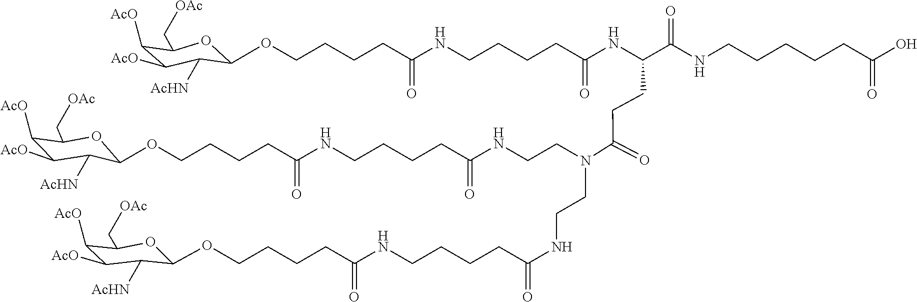 [SMILES: CC(=O)NC1C(OCCCCC(=O)NCCCCC(=O)NCCN(CCNC(=O)CCCCNC(=O)CCCCOC2OC(COC(C)=O)C(OC(C)=O)C(OC(C)=O)C2NC(C)=O)C(=O)CC[C@H](NC(=O)CCCCNC(=O)CCCCOC2OC(COC(C)=O)C(OC(C)=O)C(OC(C)=O)C2NC(C)=O)C(=O)NCCCCCC(=O)O)OC(COC(C)=O)C(OC(C)=O)C1OC(C)=O]

A round-bottom flask is charged with palladium on carbon (24 mg, 0.01 mmol, 5% by mass, 50% wet) and the flask is evacuated and backfilled with nitrogen. A solution of benzyl 6-[[(2S)-2-[5-[5-[3-acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoyl]amino]hexanoate (222 mg, 0.11 mmol) in MeOH (2.2 mL) is added via syringe followed by three drops of acetic acid. The flask is evacuated and backfilled with 1 atm hydrogen and the mixture is stirred under 1 atm hydrogen at ambient temperature. After 5 hours, the flask is purged with nitrogen and the mixture is filtered through diatomaceous earth. The filtrate is concentrated in vacuo to give the title compound (180 mg, 85%). ES/MS m/z 966.2 (M+2H)/2.

### Preparation 30

### (2,5-Dioxopyrrolidin-1-yl) 6-[[(2S)-2-[5-[5-[3-acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoyl]amino]hexanoate

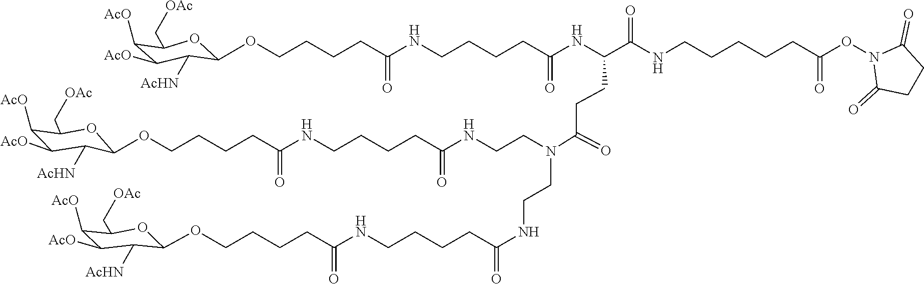 [SMILES: CC(=O)NC1C(OCCCCC(=O)NCCCCC(=O)NCCN(CCNC(=O)CCCCNC(=O)CCCCOC2OC(COC(C)=O)C(OC(C)=O)C(OC(C)=O)C2NC(C)=O)C(=O)CC[C@H](NC(=O)CCCCNC(=O)CCCCOC2OC(COC(C)=O)C(OC(C)=O)C(OC(C)=O)C2NC(C)=O)C(=O)NCCCCCC(=O)ON2C(=O)CCC2=O)OC(COC(C)=O)C(OC(C)=O)C1OC(C)=O]

The title compound is prepared from 6-[[(2S)-2-[5-[5-[3-acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoyl]amino]hexanoic acid in a manner essentially analogous to the method of preparation 16. ES/MS m/z 1014.6 (M+2H)/2.

### Preparation 31

### Benzyl 11-[[(2S)-2-[5-[5-[3-acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoyl]amino]undecanoate

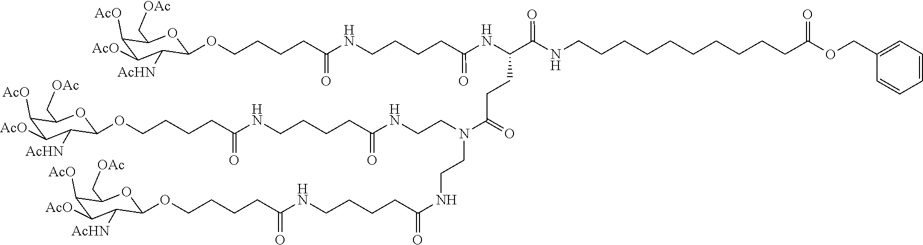 [SMILES: CC(=O)NC1C(OCCCCC(=O)NCCCCC(=O)NCCN(CCNC(=O)CCCCNC(=O)CCCCOC2OC(COC(C)=O)C(OC(C)=O)C(OC(C)=O)C2NC(C)=O)C(=O)CC[C@H](NC(=O)CCCCNC(=O)CCCCOC2OC(COC(C)=O)C(OC(C)=O)C(OC(C)=O)C2NC(C)=O)C(=O)NCCCCCCCCCCC(=O)OCc2ccccc2)OC(COC(C)=O)C(OC(C)=O)C1OC(C)=O]

The title compound is prepared from (2S)-2-[5-[5-[3-acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoic acid and benzyl 11-aminoudecanoate hydrochloride in a manner essentially analogous to the method of preparation 10. ES/MS m/z 1046.6 (M+2H)/2.

### Preparation 32

### 11-[[(2S)-2-[5-[5-[3-Acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoyl]amino]undecanoic acid

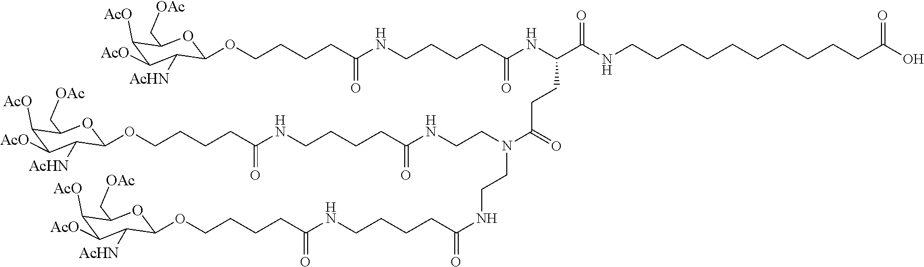 [SMILES: CC(=O)NC1C(OCCCCC(=O)NCCCCC(=O)NCCN(CCNC(=O)CCCCNC(=O)CCCCOC2OC(COC(C)=O)C(OC(C)=O)C(OC(C)=O)C2NC(C)=O)C(=O)CC[C@H](NC(=O)CCCCNC(=O)CCCCOC2OC(COC(C)=O)C(OC(C)=O)C(OC(C)=O)C2NC(C)=O)C(=O)NCCCCCCCCCCC(=O)O)OC(COC(C)=O)C(OC(C)=O)C1OC(C)=O]

To a round-bottom flask is added palladium on carbon (35 mg, 0.02 mmol, 5 mass %, 50% wet) and the flask is evacuated and backfilled with nitrogen three times. A solution of benzyl 11-[[(2S)-2-[5-[5-[3-acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoyl]amino]undecanoate (285 mg, 80% purity, 0.11 mmol) is added via syringe. The vessel is evacuated and backfilled with 1 atm hydrogen and the mixture is then stirred at ambient temperature under 1 atm hydrogen. After stirring for 3 hours, the flask is purged with nitrogen and the mixture is filtered through diatomaceous earth. The filtrate is concentrated to give the title compound (213 mg, 79% purity, 77%). ES/MS m/z 1001.20 (M+2H)/2.

### Preparation 33

### (2,5-Dioxopyrrolidin-1-yl) 11-[[(2S)-2-[5-[5-[3-acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoyl]amino]undecanoate

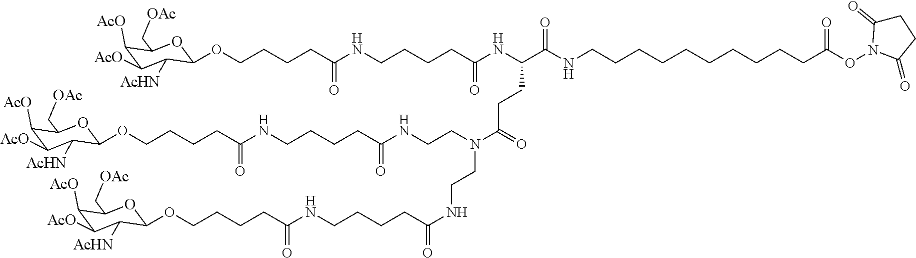 [SMILES: CC(=O)NC1C(OCCCCC(=O)NCCCCC(=O)NCCN(CCNC(=O)CCCCNC(=O)CCCCOC2OC(COC(C)=O)C(OC(C)=O)C(OC(C)=O)C2NC(C)=O)C(=O)CC[C@H](NC(=O)CCCCNC(=O)CCCCOC2OC(COC(C)=O)C(OC(C)=O)C(OC(C)=O)C2NC(C)=O)C(=O)NCCCCCCCCCCC(=O)ON2C(=O)CCC2=O)OC(COC(C)=O)C(OC(C)=O)C1OC(C)=O]

The title compound is prepared from 11-[[(2S)-2-[5-[5-[3-acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoyl]amino]undecanoic acid in a manner essentially analogous to the method of preparation 16. ES/MS m/z 1050 (M+2H)/2

### Preparation 34

### [5-Acetamido-6-[5-[2-[[(4S)-4-[5-[3-acetamido-4,5-diacetoxy-6-(acetoxymethyl)tetrahydropyran-2-yl]oxypentanoylamino]-5-[[6-[[(1S)-1-[[bis(4-methoxyphenyl)-phenyl-methoxy]methyl]-2-[4-(hydroxymethyl)-1-piperidyl]-2-oxo-ethyl]amino]-6-oxo-hexyl]amino]-5-oxo-pentanoyl]-[2-[5-[3-acetamido-4,5-diacetoxy-6-(acetoxymethyl)tetrahydropyran-2-yl]oxypentanoylamino]ethyl]amino]ethylamino]-5-oxo-pentoxy]-3,4-diacetoxy-tetrahydropyran-2-yl]methyl acetate

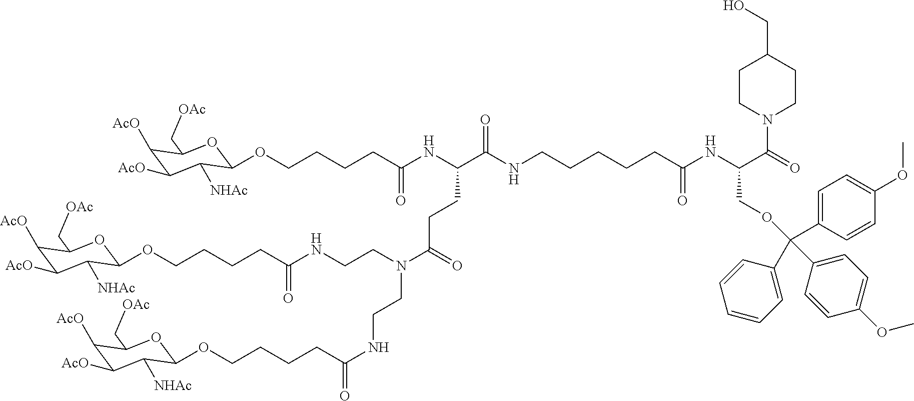 [SMILES: COc1ccc(C(OC[C@H](NC(=O)CCCCCNC(=O)[C@H](CCC(=O)N(CCNC(=O)CCCCOC2OC(COC(C)=O)C(OC(C)=O)C(OC(C)=O)C2NC(C)=O)CCNC(=O)CCCCOC2OC(COC(C)=O)C(OC(C)=O)C(OC(C)=O)C2NC(C)=O)NC(=O)CCCCOC2OC(COC(C)=O)C(OC(C)=O)C(OC(C)=O)C2NC(C)=O)C(=O)N2CCC(CO)CC2)(c2ccccc2)c2ccc(OC)cc2)cc1]

The title compound is prepared from 6-[[(2S)-2-[5-[3-acetamido-4,5-diacetoxy-6-(acetoxymethyl)tetrahydropyran-2-yl]oxypentanoylamino]-5-[bis[2-[5-[3-acetamido-4,5-diacetoxy-6-(acetoxymethyl)tetrahydropyran-2-yl]oxypentanoylamino]ethyl]amino]-5-oxo-pentanoyl]amino]hexanoic acid and (2S)-2-amino-3-[bis(4-methoxyphenyl)-phenyl-methoxy]-1-[4-(hydroxymethyl)-1-piperidyl]propan-1-one in a manner essentially analogous to the method of preparation 10. ES/MS m/z 1059.2 (M−2H)/2.

### Preparation 35

### 4-[[1-[(2S)-2-[6-[[(2S)-2-[5-[3-Acetamido-4,5-diacetoxy-6-(acetoxymethyl)tetrahydropyran-2-yl]oxypentanoylamino]-5-[bis[2-[5-[3-acetamido-4,5-diacetoxy-6-(acetoxymethyl)tetrahydropyran-2-yl]oxypentanoylamino]ethyl]amino]-5-oxo-pentanoyl]amino]hexanoylamino]-3-[bis(4-methoxyphenyl)-phenyl-methoxy]propanoyl]-4-piperidyl]methoxy]-4-oxo-butanoic acid

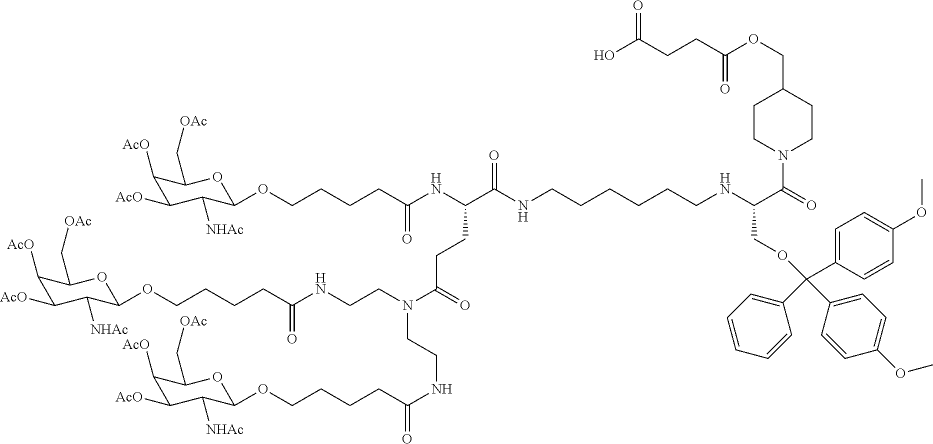 [SMILES: COc1ccc(C(OC[C@H](NCCCCCCNC(=O)[C@H](CCC(=O)N(CCNC(=O)CCCCOC2OC(COC(C)=O)C(OC(C)=O)C(OC(C)=O)C2NC(C)=O)CCNC(=O)CCCCOC2OC(COC(C)=O)C(OC(C)=O)C(OC(C)=O)C2NC(C)=O)NC(=O)CCCCOC2OC(COC(C)=O)C(OC(C)=O)C(OC(C)=O)C2NC(C)=O)C(=O)N2CCC(COC(=O)CCC(=O)O)CC2)(c2ccccc2)c2ccc(OC)cc2)cc1]

To a solution of [5-acetamido-6-[5-[2-[[(4S)-4-[5-[3-acetamido-4,5-diacetoxy-6-(acetoxymethyl)tetrahydropyran-2-yl]oxypentanoylamino]-5-[[6-[[(1S)-1-[[bis(4-methoxyphenyl)-phenyl-methoxy]methyl]-2-[4-(hydroxymethyl)-1-piperidyl]-2-oxo-ethyl]amino]-6-oxo-hexyl]amino]-5-oxo-pentanoyl]-[2-[5-[3-acetamido-4,5-diacetoxy-6-(acetoxymethyl)tetrahydropyran-2-yl]oxypentanoylamino]ethyl]amino]ethylamino]-5-oxo-pentoxy]-3,4-diacetoxy-tetrahydropyran-2-yl]methyl acetate (1.194 g, 0.56 mmol) in DCM (11 mL) is added succinic anhydride (113 mg, 1.13 mmol), TEA (0.4 mL, 3 mmol) and DMAP (213 mg, 1.69 mmol). The mixture is stirred at ambient temperature for 1 hour. After this time, the mixture is diluted with saturated NH4Cl (200 mL) and extracted with DCM (3×200 mL) and 3:1 CHCl3:IPA (200 mL). The organic layers are combined, dried over sodium sulfate, filtered, and concentrated in vacuo. The resulting residue is purified by silica gel flash chromatography eluting with 0-40% MeOH/DCM and the resulting product is dried in a vacuum oven at 40° C. for 3 hours to give the title compound (1.081 g, 86%). ES/MS m/z 1109.60 (M−2H)/2.

### Preparation 36

### Resin Loading

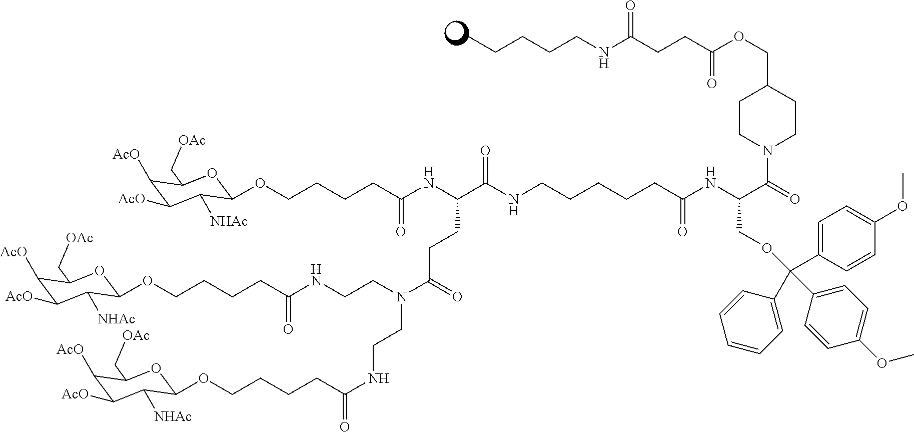 [SMILES: CCCCCNC(=O)CCC(=O)OCC1CCN(C(=O)[C@H](COC(c2ccccc2)(c2ccc(OC)cc2)c2ccc(OC)cc2)NC(=O)CCCCCNC(=O)[C@H](CCC(=O)N(CCNC(=O)CCCCOC2OC(COC(C)=O)C(OC(C)=O)C(OC(C)=O)C2NC(C)=O)CCNC(=O)CCCCOC2OC(COC(C)=O)C(OC(C)=O)C(OC(C)=O)C2NC(C)=O)NC(=O)CCCCOC2OC(COC(C)=O)C(OC(C)=O)C(OC(C)=O)C2NC(C)=O)CC1]

A solution of 4-[[1-[(2S)-2-[6-[[(2S)-2-[5-[3-acetamido-4,5-diacetoxy-6-(acetoxymethyl)tetrahydropyran-2-yl]oxypentanoylamino]-5-[bis[2-[5-[3-acetamido-4,5-diacetoxy-6-(acetoxymethyl)tetrahydropyran-2-yl]oxypentanoylamino]ethyl]amino]-5-oxo-pentanoyl]amino]hexanoylamino]-3-[bis(4-methoxyphenyl)-phenyl-methoxy]propanoyl]-4-piperidyl]methoxy]-4-oxo-butanoic acid (1.00 g, 0.61 mmol) in MeCN (6 mL) and DCM (1 mL) is transferred to a resin loading cartridge. To the vessel are added 2-(1H-benzotriazol-1-yl)-1,1,3,3-tetramethyluronium hexafluorophosphate (386 mg, 0.97 mmol) and DIEA (0.25 mL, 0.48 mmol) and the cartridge is shaken at ambient temperature for 5 minutes. After this time, 1000 Å LCAA controlled-pore glass resin (5.39 g, 90 μmol/g loading, purchased from ChemGenes) is added and the mixture is shaken at ambient temperature for 18 hours. After this time, the cartridge is drained by suction and the resin is washed by shaking with DCM (10 mL) for 10 minutes. The cartridge is drained and the washing and draining procedure is repeated with 10% MeOH/DCM (10 mL) and Et2O (10 mL). After draining, a solution of acetic anhydride (6.4 mL), pyridine (20 mL) and TEA (0.22 mL) is added and the cartridge is shaken for 2 hours. After this time, the cartridge is drained and the washing and draining procedure above is repeated using DCM (10 mL), 10% MeOH/DCM (10 mL) and diethyl ether (10 mL). After draining, the resin is dried under vacuum for 30 minutes. The resin loading is determined using a standard trityl assay. The resin loading was calculated to be 34.7 μmol/g.

### Preparation 37

### Benzyl 2-[2-[[(2S)-2-[5-[5-[3-acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoyl]amino]ethoxy]acetate

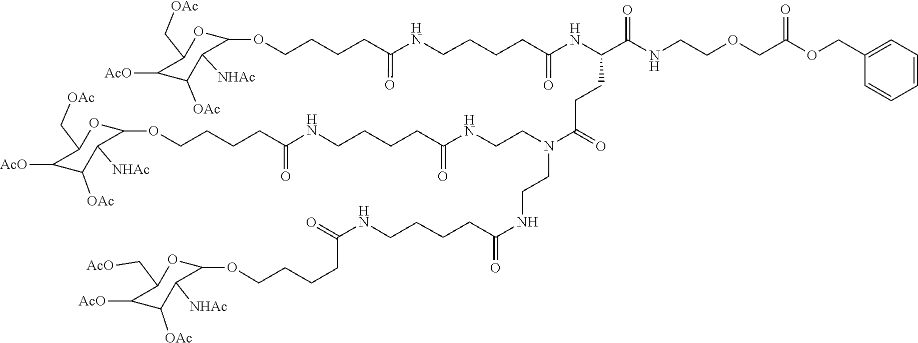 [SMILES: CC(=O)NC1C(OCCCCC(=O)NCCCCC(=O)NCCN(CCNC(=O)CCCCNC(=O)CCCCOC2OC(COC(C)=O)C(OC(C)=O)C(OC(C)=O)C2NC(C)=O)C(=O)CC[C@H](NC(=O)CCCCNC(=O)CCCCOC2OC(COC(C)=O)C(OC(C)=O)C(OC(C)=O)C2NC(C)=O)C(=O)NCCOCC(=O)OCc2ccccc2)OC(COC(C)=O)C(OC(C)=O)C1OC(C)=O]

The title compound is prepared from (2S)-2-[5-[5-[3-Acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoic acid and benzyl 2-(2-aminoethoxy)acetate hydrochloride in a manner essentially analogous to the method of preparation 10. ES/MS m/z 1005.2 (M+2H/2).

### Preparation 38

### 2-[2-[[(2S)-2-[5-[5-[3-Acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoyl]amino]ethoxy]acetic acid

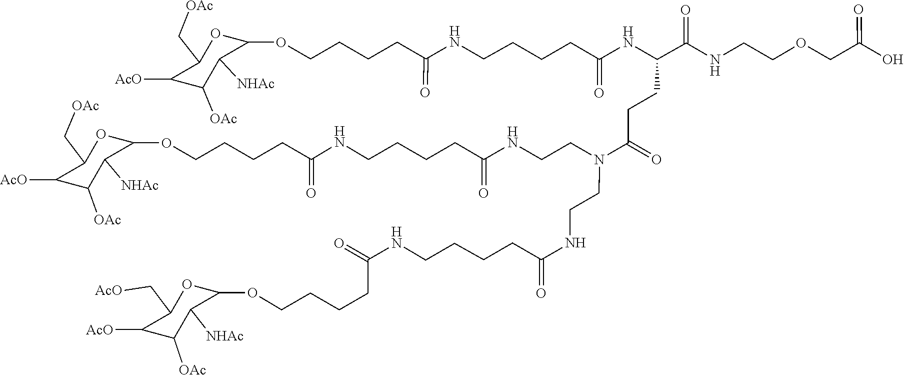 [SMILES: CC(=O)NC1C(OCCCCC(=O)NCCCCC(=O)NCCN(CCNC(=O)CCCCNC(=O)CCCCOC2OC(COC(C)=O)C(OC(C)=O)C(OC(C)=O)C2NC(C)=O)C(=O)CC[C@H](NC(=O)CCCCNC(=O)CCCCOC2OC(COC(C)=O)C(OC(C)=O)C(OC(C)=O)C2NC(C)=O)C(=O)NCCOCC(=O)O)OC(COC(C)=O)C(OC(C)=O)C1OC(C)=O]

Benzyl 2-[2-[[(2S)-2-[5-[5-[3-acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoyl]amino]ethoxy]acetate (0.120 mmol, 240 mg) is combined with 5% Pd/C (1.17 mmol, 124 mg) in MeOH (12.0 ml). The mixture is hydrogenated on a Parr shaker (ambient temperature, 10 psi) for 48 minutes, filtered through diatomaceous earth, and concentrated in vacuo to give the title compound as a gray solid (187 mg, 82%). ES/MS m/z 960.0 (M+2H/2).

### Preparation 39

### (2,3,5,6-Tetrafluorophenyl) 2-[2-[[(2S)-2-[5-[5-[3-acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoyl]amino]ethoxy]acetate

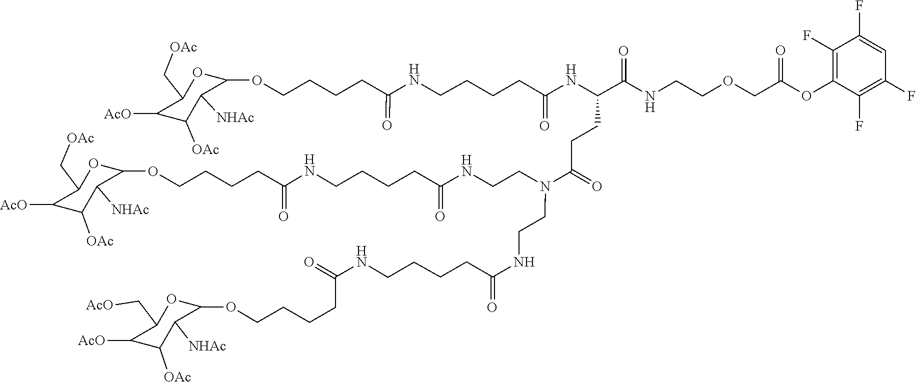 [SMILES: CC(=O)NC1C(OCCCCC(=O)NCCCCC(=O)NCCN(CCNC(=O)CCCCNC(=O)CCCCOC2OC(COC(C)=O)C(OC(C)=O)C(OC(C)=O)C2NC(C)=O)C(=O)CC[C@H](NC(=O)CCCCNC(=O)CCCCOC2OC(COC(C)=O)C(OC(C)=O)C(OC(C)=O)C2NC(C)=O)C(=O)NCCOCC(=O)Oc2c(F)c(F)cc(F)c2F)OC(COC(C)=O)C(OC(C)=O)C1OC(C)=O]

To 2-[2-[[(2S)-2-[5-[5-[3-acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoyl]amino]ethoxy]acetic acid (0.096 mmol, 184 mg) and DIEA (0.765 mmol, 140 μL) in DCM (3.0 ml) is added (2,3,5,6-tetrafluorophenyl) 2,2,2-trifluoroacetate (0.383 mmol, 100 mg) to the mixture dropwise. The mixture is stirred at ambient temperature for 16 hours. The reaction mixture is purified directly by silica gel flash chromatography eluting with 0% to 50% MeOH/DCM to give the title compound as a tan solid (197 mg, 99%). ES/MS m/z 1034.0 (M+2H/2).

### Preparation 40

### Benzyl 2-[2-[2-[2-[[(2S)-2-[5-[5-[3-acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoyl]amino]ethoxy]ethoxy]ethoxy]acetate

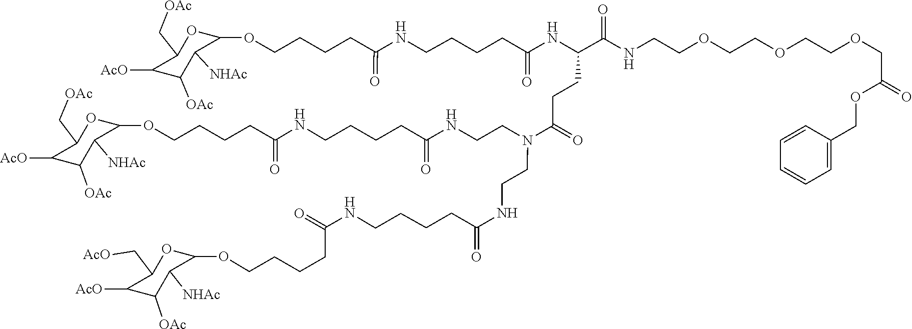 [SMILES: CC(=O)NC1C(OCCCCC(=O)NCCCCC(=O)NCCN(CCNC(=O)CCCCNC(=O)CCCCOC2OC(COC(C)=O)C(OC(C)=O)C(OC(C)=O)C2NC(C)=O)C(=O)CC[C@H](NC(=O)CCCCNC(=O)CCCCOC2OC(COC(C)=O)C(OC(C)=O)C(OC(C)=O)C2NC(C)=O)C(=O)NCCOCCOCCOCC(=O)OCc2ccccc2)OC(COC(C)=O)C(OC(C)=O)C1OC(C)=O]

The title compound is prepared from (2S)-2-[5-[5-[3-acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoic acid and benzyl 2-[2-[2-(2-aminoethoxy)ethoxy]ethoxy]acetate hydrochloride in a manner essentially analogous to the method of preparation 10. ES/MS m/z 1049.0 (M+2H/2).

### Preparation 41

### 2-[2-[2-[2-[[(2S)-2-[5-[5-[3-Acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoyl]amino]ethoxy]ethoxy]ethoxy]acetic acid

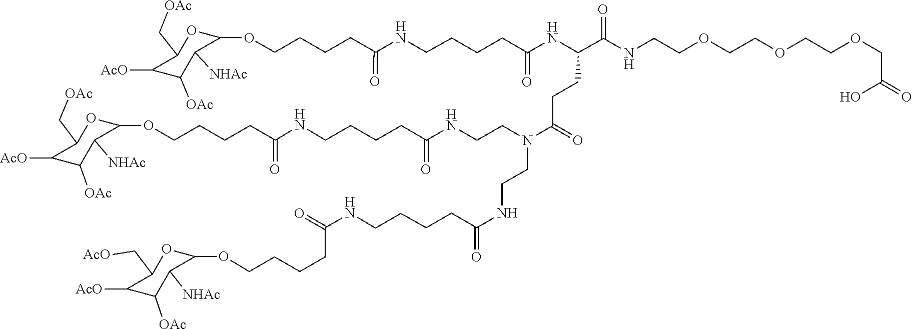 [SMILES: CC(=O)NC1C(OCCCCC(=O)NCCCCC(=O)NCCN(CCNC(=O)CCCCNC(=O)CCCCOC2OC(COC(C)=O)C(OC(C)=O)C(OC(C)=O)C2NC(C)=O)C(=O)CC[C@H](NC(=O)CCCCNC(=O)CCCCOC2OC(COC(C)=O)C(OC(C)=O)C(OC(C)=O)C2NC(C)=O)C(=O)NCCOCCOCCOCC(=O)O)OC(COC(C)=O)C(OC(C)=O)C1OC(C)=O]

Benzyl 2-[2-[2-[2-[[(2S)-2-[5-[5-[3-acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoyl]amino]ethoxy]ethoxy]ethoxy]acetate (0.118 mmol, 247 mg) is combined with 5% Pd/C (1.17 mmol, 124 mg) in MeOH (12.0 mL). The mixture is hydrogenated on a Parr shaker (ambient temperature, 10 psi) for 1 hour, filtered through diatomaceous earth, and concentrated in vacuo to give the title compound as a gray solid (227 mg, 96%). ES/MS m/z 1004.0 (M+2H/2).

### Preparation 42

### (2,3,5,6-Tetrafluorophenyl) 2-[2-[2-[2-[[(2S)-2-[5-[5-[3-acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoyl]amino]ethoxy]ethoxy]ethoxy]acetate

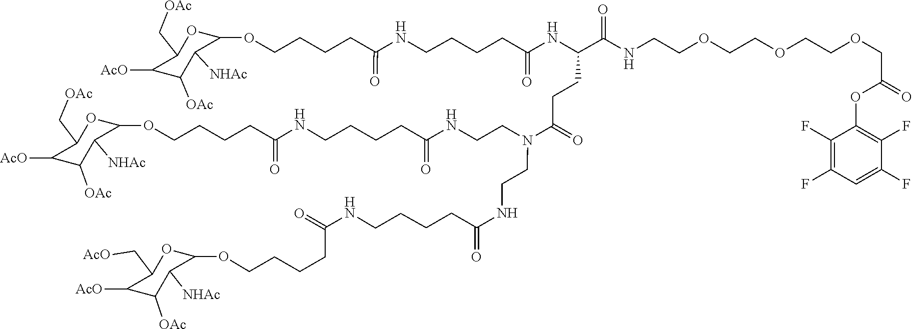 [SMILES: CC(=O)NC1C(OCCCCC(=O)NCCCCC(=O)NCCN(CCNC(=O)CCCCNC(=O)CCCCOC2OC(COC(C)=O)C(OC(C)=O)C(OC(C)=O)C2NC(C)=O)C(=O)CC[C@H](NC(=O)CCCCNC(=O)CCCCOC2OC(COC(C)=O)C(OC(C)=O)C(OC(C)=O)C2NC(C)=O)C(=O)NCCOCCOCCOCC(=O)Oc2c(F)c(F)cc(F)c2F)OC(COC(C)=O)C(OC(C)=O)C1OC(C)=O]

To 2-[2-[2-[2-[[(2S)-2-[5-[5-[3-Acetamido-4,5-diacetoxy-6-(acetoxymethyl)tetrahydropyran-2-yl]oxypentanoylamino]pentanoylamino]-5-[bis[2-[5-[5-[3-acetamido-4,5-diacetoxy-6-(acetoxymethyl)tetrahydropyran-2-yl]oxypentanoylamino]pentanoylamino]ethyl]amino]-5-oxo-pentanoyl]amino]ethoxy]ethoxy]ethoxy]acetic acid (0.111 mmol, 222 mg) and DIEA (0.883 mmol, 154 μL) in DCM (3.0 ml) is added (2,3,5,6-tetrafluorophenyl) 2,2,2-trifluoroacetate (0.443 mmol, 116 mg) to the mixture dropwise. The mixture is stirred at ambient temperature for 16 hours. The reaction mixture is purified directly by silica gel flash chromatography eluting with 0% to 50% MeOH/DCM to give the title compound as a tan solid (174 mg, 73%). ES/MS m/z 1078.2 (M+2H/2).

### Example 1: Conjugation Protocol

For the synthesis of GalNAc-conjugated sense strands, a sense strand with a 3′ C6-NH2 functional group was first synthesized using standard phosphoramidite chemistry. A stock solution of GalNAc ligand-NHS ester (10 mmol/L in acetonitrile; 1 eq) was prepared. Borate buffer (10% v/v; 20×) was added to oligonucleotide C6-NH2 sense strand in an Eppendorf tube, then GalNAc ligand (5 eq) was added. The mixture was shaken at ambient temperature for 16 hours. After this time, the mixture was transferred to a 15 mL falcon tube, ammonium hydroxide (28 mass %) was added, and the mixture was shaken at ambient temperature for 2 hours. The ammonia was then removed in vacuo. The residue was purified by ion-exchange chromatography. Conditions: Solvent A: 15% MeCN/20 mM NaH2PO4, Solvent B: 15% MeCN/20 mM NaH2PO4, 1M NaBr; 35-55% B over 5 CV at 8 mL/min, column temperature 60° C. The desired fractions were pooled and desalted by spin-filtration using an Eppendorf centrifuge or desalting column. After desalting, the material was recovered and OD and volume were measured to obtain concentration.

Alternatively, conjugation was to the 5′ position of the sense strand through immobilizing the GalNAc ligand on microporous polystyrene resin or controlled pore glass and synthesizing using established solid phase oligonucleotide synthesis methods with 5′-CE β-cyanoethyl) phosphoramidites.

Alternatively, the GalNAc ligand was converted to a suitable phosphoramidite and delivered to the 5′ position of the sense strand using standard phosphoramidite chemistry.

### Example 2: Annealing

To generate the siRNA duplexes of a sense and antisense strand, the following procedures were performed. To a falcon tube containing oligonucleotide sense strand-GalNAc conjugate, the corresponding antisense oligonucleotide (1 eq) was added and vortexed for 10 seconds before spin-filtering through 100K MWCO Amicon filter unit to remove particulates. The filtrate was recovered and concentrated in vacuo on a Genevac evaporator. The residue was reconstituted in 1×PBS, filtered through 0.2μ filter, and OD and volume were measured to obtain concentration.

An endotoxin test was performed using a Limulus amebocyte lysate on an Endosafe®-nexgen PTS instrument.

TABLE 1
Exemplary molecules synthesized ustilizing the aforementioned conjugation
and annealing protocols.

| Molecule | |
|---|---|
| Identifier | Ligand attached to 3′ of sense strand |
| 1 | 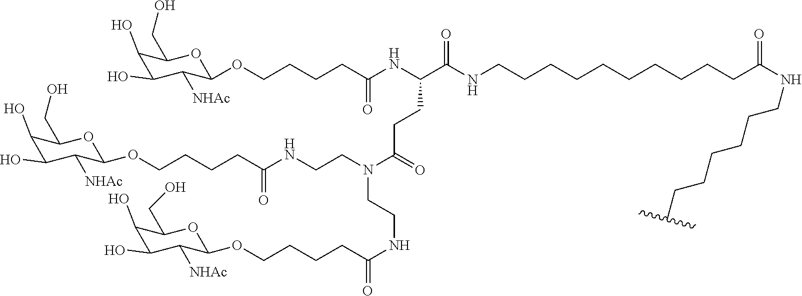 [SMILES: *CCCCCCNC(=O)CCCCCCCCCCNC(=O)[C@H](CCC(=O)N(CCNC(=O)CCCCOC1OC(CO)C(O)C(O)C1NC(C)=O)CCNC(=O)CCCCOC1OC(CO)C(O)C(O)C1NC(C)=O)NC(=O)CCCCOC1OC(CO)C(O)C(O)C1NC(C)=O] |
| 2 | 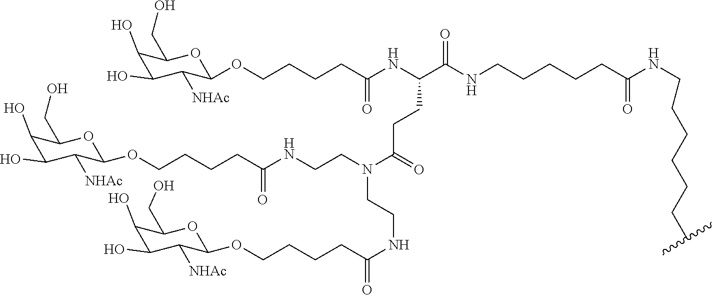 [SMILES: *CCCCCCNC(=O)CCCCCNC(=O)[C@H](CCC(=O)N(CCNC(=O)CCCCOC1OC(CO)C(O)C(O)C1NC(C)=O)CCNC(=O)CCCCOC1OC(CO)C(O)C(O)C1NC(C)=O)NC(=O)CCCCOC1OC(CO)C(O)C(O)C1NC(C)=O] |
| 3 | 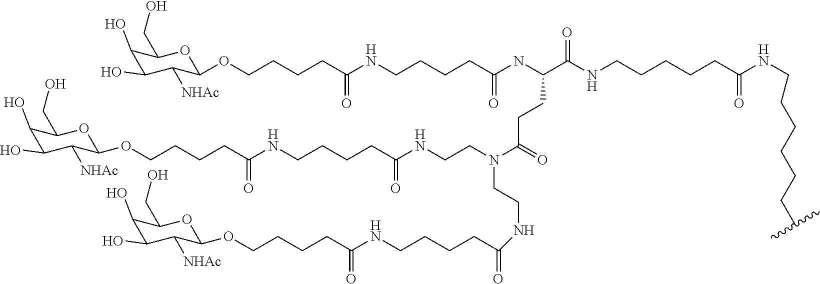 [SMILES: *CCCCCCNC(=O)CCCCCNC(=O)[C@H](CCC(=O)N(CCNC(=O)CCCCNC(=O)CCCCOC1OC(CO)C(O)C(O)C1NC(C)=O)CCNC(=O)CCCCNC(=O)CCCCOC1OC(CO)C(O)C(O)C1NC(C)=O)NC(=O)CCCCNC(=O)CCCCOC1OC(CO)C(O)C(O)C1NC(C)=O] |
| 4 | 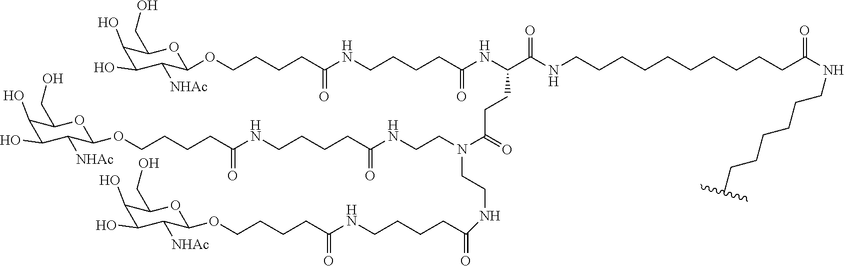 [SMILES: *CCCCCCNC(=O)CCCCCCCCCCNC(=O)[C@H](CCC(=O)N(CCNC(=O)CCCCNC(=O)CCCCOC1OC(CO)C(O)C(O)C1NC(C)=O)CCNC(=O)CCCCNC(=O)CCCCOC1OC(CO)C(O)C(O)C1NC(C)=O)NC(=O)CCCCNC(=O)CCCCOC1OC(CO)C(O)C(O)C1NC(C)=O] |
| 5 | 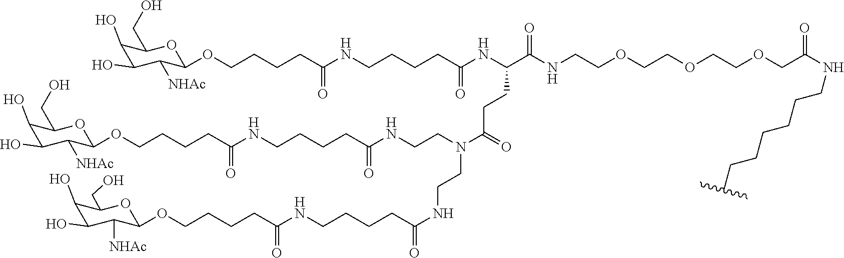 [SMILES: *CCCCCCNC(=O)COCCOCCOCCNC(=O)[C@H](CCC(=O)N(CCNC(=O)CCCCNC(=O)CCCCOC1OC(CO)C(O)C(O)C1NC(C)=O)CCNC(=O)CCCCNC(=O)CCCCOC1OC(CO)C(O)C(O)C1NC(C)=O)NC(=O)CCCCNC(=O)CCCCOC1OC(CO)C(O)C(O)C1NC(C)=O] |
| 6 | 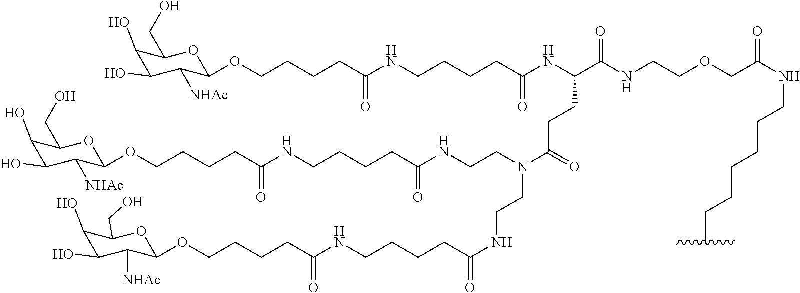 [SMILES: *CCCCCCNC(=O)COCCNC(=O)[C@H](CCC(=O)N(CCNC(=O)CCCCNC(=O)CCCCOC1OC(CO)C(O)C(O)C1NC(C)=O)CCNC(=O)CCCCNC(=O)CCCCOC1OC(CO)C(O)C(O)C1NC(C)=O)NC(=O)CCCCNC(=O)CCCCOC1OC(CO)C(O)C(O)C1NC(C)=O] |

### Example 3

### General Procedure for Oligo Synthesis Using GalNAc-Functionalized CPG

Oligo synthesis was conducted on a MerMade™ 12 instrument using phosphoramidite chemistry. Sense strands were synthesized from the prefunctionalized GalNAc solid support and antisense strands were synthesized using standard support preloaded with the first nucleotide of the oligo sequence. Oligos were cleaved and deprotected using concentrated ammonium hydroxide solution (28% by mass) and purified by ion exchange chromatography using conditions described above. Desalting, annealing, and endotoxin testing were conducted.

The sequence of antisense oligonucleotides were designed using 15 to 50 nucleotides of the following FAS transcript (SEQ ID NO: 1), where T nucleotides were replaced by U nucleotides, and where one or more nucleotides and one or more internucleotide linkages were optionally further modified as described herein.

Homo sapiens FAS Cell Death Receptor (FAS) Transcript,

| SEQ ID NO: 1 | |
|---|---|
| 1 | ctcttctccc gcgggttggt ggacccgctc agtacggagt tggggaagct ctttcacttc |
| 61 | ggaggattgc tcaacaacca tgctgggcat ctggaccctc ctacctctgg ttcttacgtc |
| 121 | tgttgctaga ttatcgtcca aaagtgttaa tgcccaagtg actgacatca actccaaggg |
| 181 | attggaattg aggaagactg ttactacagt tgagactcag aacttggaag gcctgcatca |
| 241 | tgatggccaa ttctgccata agccctgtcc tccaggtgaa aggaaagcta gggactgcac |
| 301 | agtcaatggg gatgaaccag actgcgtgcc ctgccaagaa gggaaggagt acacagacaa |
| 361 | agcccatttt tcttccaaat gcagaagatg tagattgtgt gatgaaggac atggcttaga |
| 421 | agtggaaata aactgcaccc ggacccagaa taccaagtgc agatgtaaac caaacttttt |
| 481 | ttgtaactct actgtatgtg aacactgtga cccttgcacc aaatgtgaac atggaatcat |
| 541 | caaggaatgc acactcacca gcaacaccaa gtgcaaagag gaaggatcca gatctaactt |
| 601 | ggggtggctt tgtcttcttc ttttgccaat tccactaatt gtttgggtga agagaaagga |
| 661 | agtacagaaa acatgcagaa agcacagaaa ggaaaaccaa ggttctcatg aatctccaac |
| 721 | tttaaatcct gaaacagtgg caataaattt atctgatgtt gacttgagta aatatatcac |
| 781 | cactattgct ggagtcatga cactaagtca agttaaaggc tttgttcgaa agaatggtgt |
| 841 | caatgaagcc aaaatagatg agatcaagaa tgacaatgtc caagacacag cagaacagaa |
| 901 | agttcaactg cttcgtaatt ggcatcaact tcatggaaag aaagaagcgt atgacacatt |
| 961 | gattaaagat ctcaaaaaag ccaatctttg tactcttgca gagaaaattc agactatcat |
| 1021 | cctcaaggac attactagtg actcagaaaa ttcaaacttc agaaatgaaa tccaaagctt |
| 1081 | ggtctagagt gaaaaacaac aaattcagtt ctgagtatat gcaattagtg tttgaaaaga |
| 1141 | ttcttaatag ctggctgtaa atactgcttg gttttttact gggtacattt tatcatttat |
| 1201 | tagcgctgaa gagccaacat atttgtagat ttttaatatc tcatgattct gcctccaagg |
| 1261 | atgtttaaaa tctagttggg aaaacaaact tcatcaagag taaatgcagt ggcatgctaa |
| 1321 | gtacccaaat aggagtgtat gcagaggatg aaagattaag attatgctct ggcatctaac |
| 1381 | atatgattct gtagtatgaa tgtaatcagt gtatgttagt acaaatgtct atccacaggc |
| 1441 | taaccccact ctatgaatca atagaagaag ctatgacctt ttgctgaaat atcagttact |
| 1501 | gaacaggcag gccactttgc ctctaaatta cctctgataa ttctagagat tttaccatat |
| 1561 | ttctaaactt tgtttataac tctgagaaga tcatatttat gtaaagtata tgtatttgag |
| 1621 | tgcagaattt aaataaggct ctacctcaaa gacctttgca cagtttattg gtgtcatatt |
| 1681 | atacaatatt tcaattgtga attcacatag aaaacattaa attataatgt ttgactatta |
| 1741 | tatatgtgta tgcattttac tggctcaaaa ctacctactt ctttctcagg catcaaaagc |
| 1801 | attttgagca ggagagtatt actagagctt tgccacctct ccatttttgc cttggtgctc |
| 1861 | atcttaatgg cctaatgcac ccccaaacat ggaaatatca ccaaaaaata cttaatagtc |
| 1921 | caccaaaagg caagactgcc cttagaaatt ctagcctggt ttggagatac taactgctct |
| 1981 | cagagaaagt agctttgtga catgtcatga acccatgttt gcaatcaaag atgataaaat |
| 2041 | agattcttat ttttccccca cccccgaaaa tgttcaataa tgtcccatgt aaaacctgct |
| 2101 | acaaatggca gcttatacat agcaatggta aaatcatcat ctggatttag gaattgctct |
| 2161 | tgtcataccc ccaagtttct aagatttaag attctcctta ctactatcct acgtttaaat |
| 2221 | atctttgaaa gtttgtatta aatgtgaatt ttaagaaata atatttatat ttctgtaaat |
| 2281 | gtaaactgtg aagatagtta taaactgaag cagatacctg gaaccaccta aagaacttcc |
| 2341 | atttatggag gatttttttg ccccttgtgt ttggaattat aaaatatagg taaaagtacg |
| 2401 | taattaaata atgtttttgg tatttctggt tttctctttt ttggtagggg cttgcttttt |
| 2461 | ggttttgtct tccttttctc taactgatgc taaatataac ttgtctttaa tgcttcttgg |
| 2521 | atcccttaga aggtacttcc tttttaacct taaccctttt agtagttaaa taattatttc |
| 2581 | cataggttgc tattgccaag aagacctctt ccaaacagca catgattatt cgtcaaacag |
| 2641 | tttcgtattc cagatactgg aatgtggata agaaagtata catttcaagg ggtaggtttt |
| 2701 | attattaaga aagccaaatg aggattttga aatattcttt cctgcatatt atccattcta |
| 2761 | gctacatgct ggccagtggg ccacctttct tttctgcaat ttaatgctag taatatattc |
| 2821 | tatttaaccc atgagtccca aagtattagc atttcaacat gtaagcatgt cggtaagata |
| 2881 | gttgtgcttt gcttagggtt ccctcctgtg ttatggtctg gaaagtgtct ttaggcagaa |
| 2941 | agtctgagtg atcacagggt tcactcatta atttctcttt tctgagccat catagtctgt |
| 3001 | gctgtctgct ctccagtttt ctatttctag acagaagtag ggcaagttag gtactagtta |
| 3061 | ttcttcatgg ccagaagtgc aagttctact ttgcaagaca agattaagtt agagaacacc |
| 3121 | ctattccact ttggtgaact cagagcaaga actttgagtt cctttgggag gaagacagtg |
| 3181 | gagaagtctt tgtacttggt gatgtggttt ttttcctcat ggcttcacct agtggcccca |
| 3241 | agcatgactt ctcccatgtc aatgagcaca gccacattcc cgagttgagg tgaccccacg |
| 3301 | gtccagaatc atcctcattc tggtgaacct ggttctcttt gtggtgggca tactgggtag |
| 3361 | gagaatcacc caaaggtcac ccatgagctg cagaaaaaaa ggctatttgc agaaggagct |
| 3421 | cacagatcac attgaaagca ttgcatattc aaacatcttg gtcttcttta ttggcatgcc |
| 3481 | cacagggtct tctgacctct gattagatca gacacttttt agatattgaa tcatcagttt |
| 3541 | ctgtacaact atctgaataa ggtatataat caatgaaatt tagaattttt ttctatgctt |
| 3601 | actcctgatt ggtaatttgt ttgggtttag aattctatac aaggccattt gtaattttcc |
| 3661 | tcagcacttt aaaaatatta aaccatgttt tcttaa |

Exemplary antisense strand sequences of 18 nucleotides in length are shown in Table 2 below, which may be optionally further modified and synthesized and incorporated into the RNAi agents, as described herein.

TABLE 2
Antisense 18 mers of FAS RNAi agents

| | AntiSense 18mer |
|---|---|
| SEQ ID NO: | 5′ to 3′ |
| SEQ ID NO: 2 | UCUAAGCCAUGUCCUUCA |
| SEQ ID NO: 3 | UACAAAAAAAGUUUGGUU |
| SEQ ID NO: 4 | AUCACACAAUCUACAUCU |
| SEQ ID NO: 5 | AUAUAUUUACUCAAGUCA |
| SEQ ID NO: 6 | UGGACAUUGUCAUUCUUG |
| SEQ ID NO: 7 | UAUAUUUACUCAAGUCAA |
| SEQ ID NO: 8 | UUGAUCUCAUCUAUUUUG |
| SEQ ID NO: 9 | CAAUCUACAUCUUCUGCA |
| SEQ ID NO: 10 | ACCAUUCUUUCGAACAAA |
| SEQ ID NO: 11 | GUGAUAUAUUUACUCAAG |
| SEQ ID NO: 12 | UGUCAUUCUUGAUCUCAU |
| SEQ ID NO: 13 | GUGGUGAUAUAUUUACUC |
| SEQ ID NO: 14 | CAUUCUUGAUCUCAUCUA |
| SEQ ID NO: 15 | CACCAUUCUUUCGAACAA |
| SEQ ID NO: 16 | AAGCCAUGUCCUUCAUCA |
| SEQ ID NO: 17 | UGAUAUAUUUACUCAAGU |
| SEQ ID NO: 18 | UACGAAGCAGUUGAACUU |
| SEQ ID NO: 19 | ACACAAUCUACAUCUUCU |
| SEQ ID NO: 20 | CAAGGGUCACAGUGUUCA |
| SEQ ID NO: 21 | CUUUAACUUGACUUAGUG |
| SEQ ID NO: 22 | UACAUCUGCACUUGGUAU |
| SEQ ID NO: 23 | GCUGUGUCUUGGACAUUG |
| SEQ ID NO: 24 | UUCUAAGCCAUGUCCUUC |
| SEQ ID NO: 25 | UACAUCUUCUGCAUUUGG |
| SEQ ID NO: 26 | ACAUUGUCAUUCUUGAUC |
| SEQ ID NO: 27 | UUGGUUUACAUCUGCACU |
| SEQ ID NO: 28 | ACACCAUUCUUUCGAACA |
| SEQ ID NO: 29 | CACACAAUCUACAUCUUC |
| SEQ ID NO: 30 | UCUCUGCAAGAGUACAAA |
| SEQ ID NO: 31 | UUGGUGCAAGGGUCACAG |
| SEQ ID NO: 32 | CUACAUCUUCUGCAUUUG |
| SEQ ID NO: 33 | UCUGCUGUGUCUUGGACA |
| SEQ ID NO: 34 | GUACUCCUUCCCUUCUUG |
| SEQ ID NO: 35 | UGGUGAUAUAUUUACUCA |
| SEQ ID NO: 36 | GUCAUUCUUGAUCUCAUC |
| SEQ ID NO: 37 | GAUAUAUUUACUCAAGUC |
| SEQ ID NO: 38 | AUAGUGGUGAUAUAUUUA |
| SEQ ID NO: 39 | GUUUACAUCUGCACUUGG |
| SEQ ID NO: 40 | GUCACAGUGUUCACAUAC |
| SEQ ID NO: 41 | GGACAUUGUCAUUCUUGA |
| SEQ ID NO: 42 | UUAACUUGACUUAGUGUC |
| SEQ ID NO: 43 | AUUCUUGAUCUCAUCUAU |
| SEQ ID NO: 44 | GGGUCACAGUGUUCACAU |
| SEQ ID NO: 45 | UUACAUCUGCACUUGGUA |
| SEQ ID NO: 46 | CAUUGUCAUUCUUGAUCU |
| SEQ ID NO: 47 | UUCUCUGCAAGAGUACAA |
| SEQ ID NO: 48 | UCUCAUCUAUUUUGGCUU |
| SEQ ID NO: 49 | UAGUAAUGUCCUUGAGGA |
| SEQ ID NO: 50 | AGGGUCACAGUGUUCACA |
| SEQ ID NO: 51 | AGUCACUUGGGCAUUAAC |
| SEQ ID NO: 52 | CCAAUUACGAAGCAGUUG |
| SEQ ID NO: 53 | CACAAUCUACAUCUUCUG |
| SEQ ID NO: 54 | UAGUGGUGAUAUAUUUAC |
| SEQ ID NO: 55 | UGGUGAGUGUGCAUUCCU |
| SEQ ID NO: 56 | CCUAGCUUUCCUUUCACC |
| SEQ ID NO: 57 | GUGUCUUGGACAUUGUCA |
| SEQ ID NO: 58 | UGCUGGUGAGUGUGCAUU |
| SEQ ID NO: 59 | CCUUUCACCUGGAGGACA |
| SEQ ID NO: 60 | ACAAUCUACAUCUUCUGC |
| SEQ ID NO: 61 | CCAUGUCCUUCAUCACAC |
| SEQ ID NO: 62 | UUACGAAGCAGUUGAACU |
| SEQ ID NO: 63 | CAGUCACUUGGGCAUUAA |
| SEQ ID NO: 64 | UAUUUACUCAAGUCAACA |
| SEQ ID NO: 65 | AUCUCAUCUAUUUUGGCU |
| SEQ ID NO: 66 | CAAUUACGAAGCAGUUGA |
| SEQ ID NO: 67 | AAUUUUCUCUGCAAGAGU |
| SEQ ID NO: 68 | UCACUAGUAAUGUCCUUG |
| SEQ ID NO: 69 | UGUCUGUGUACUCCUUCC |
| SEQ ID NO: 70 | AACUUGACUUAGUGUCAU |
| SEQ ID NO: 71 | CACUAGUAAUGUCCUUGA |
| SEQ ID NO: 72 | CCAUUCUUUCGAACAAAG |
| SEQ ID NO: 73 | UGGCAGGGCACGCAGUCU |
| SEQ ID NO: 74 | UGAUCUCAUCUAUUUUGG |
| SEQ ID NO: 75 | CUGUGUACUCCUUCCCUU |
| SEQ ID NO: 76 | UGUCCUUCAUCACACAAU |
| SEQ ID NO: 77 | AGCAAUCCUCCGAAGUGA |
| SEQ ID NO: 78 | UGCAGUCCCUAGCUUUCC |
| SEQ ID NO: 79 | UUCCACUUCUAAGCCAUG |
| SEQ ID NO: 80 | AUUUUCUCUGCAAGAGUA |
| SEQ ID NO: 81 | GGGCACGCAGUCUGGUUC |
| SEQ ID NO: 82 | UAACUUGACUUAGUGUCA |
| SEQ ID NO: 83 | GACACCAUUCUUUCGAAC |
| SEQ ID NO: 84 | UGGUGCAAGGGUCACAGU |
| SEQ ID NO: 85 | UCCGGGUGCAGUUUAUUU |
| SEQ ID NO: 86 | UCACUUGGGCAUUAACAC |
| SEQ ID NO: 87 | AAUUACGAAGCAGUUGAA |
| SEQ ID NO: 88 | UUGAGCAAUCCUCCGAAG |
| SEQ ID NO: 89 | CUCAUCUAUUUUGGCUUC |
| SEQ ID NO: 90 | GUUGAGCAAUCCUCCGAA |
| SEQ ID NO: 91 | CCCUAGCUUUCCUUUCAC |
| SEQ ID NO: 92 | GUCUGUGUACUCCUUCCC |
| SEQ ID NO: 93 | GGGUGCAGUUUAUUUCCA |
| SEQ ID NO: 94 | UGUGUCUUGGACAUUGUC |
| SEQ ID NO: 95 | AUGUCCUUCAUCACACAA |
| SEQ ID NO: 96 | ACGCAGUCUGGUUCAUCC |
| SEQ ID NO: 97 | UCAGUCACUUGGGCAUUA |
| SEQ ID NO: 98 | GGCAGGGCACGCAGUCUG |
| SEQ ID NO: 99 | GGUGCAAGGGUCACAGUG |
| SEQ ID NO: 100 | UUUGUCUGUGUACUCCUU |
| SEQ ID NO: 101 | CACGCAGUCUGGUUCAUC |
| SEQ ID NO: 102 | CUUCUUGGCAGGGCACGC |
| SEQ ID NO: 103 | UGCAAGGGUCACAGUGUU |
| SEQ ID NO: 104 | CAUGGUUGUUGAGCAAUC |
| SEQ ID NO: 105 | UUUAACUUGACUUAGUGU |
| SEQ ID NO: 106 | AGUCCCUAGCUUUCCUUU |
| SEQ ID NO: 107 | UGCUGUGUCUUGGACAUU |
| SEQ ID NO: 108 | GUCCCUAGCUUUCCUUUC |
| SEQ ID NO: 109 | UCCUCCGAAGUGAAAGAG |
| SEQ ID NO: 110 | AUCUUCUGCAUUUGGAAG |
| SEQ ID NO: 111 | UGGUUGUUGAGCAAUCCU |
| SEQ ID NO: 112 | UACUCCUUCCCUUCUUGG |

TABLE 3A
Exemplary full-length sense and antisense strands of FAS RNAi agents

| | SEQ ID | | SEQ ID | |
|---|---|---|---|---|
| Duplex | NO: | Sense Strand | NO: | Antisense Strand |
| D: 1 | SEQ ID | GAUGAAGGACAUGGCUUAGAA | SEQ ID | UUCUAAGCCAUGUCCUUCAUCAC |
| | NO: | | NO: 224 | |
| | 113 | | | |
| D: 2 | SEQ ID | UAAACCAAACUUUUUUUGUAA | SEQ ID | UUACAAAAAAAGUUUGGUUUACA |
| | NO: | | NO: 225 | |
| | 114 | | | |
| D: 3 | SEQ ID | GAAGAUGUAGAUUGUGUGAUA | SEQ ID | UAUCACACAAUCUACAUCUUCUG |
| | NO: | | NO: 226 | |
| | 115 | | | |
| D: 4 | SEQ ID | GUUGACUUGAGUAAAUAUAUA | SEQ ID | UAUAUAUUUACUCAAGUCAACAU |
| | NO: | | NO: 227 | |
| | 116 | | | |
| D: 5 | SEQ ID | AUCAAGAAUGACAAUGUCCAA | SEQ ID | UUGGACAUUGUCAUUCUUGAUCU |
| | NO: | | NO: 228 | |
| | 117 | | | |
| D: 6 | SEQ ID | UGUUGACUUGAGUAAAUAUAA | SEQ ID | UUAUAUUUACUCAAGUCAACAUC |
| | NO: | | NO: 229 | |
| | 118 | | | |
| D: 7 | SEQ ID | GCCAAAAUAGAUGAGAUCAAA | SEQ ID | UUUGAUCUCAUCUAUUUUGGCUU |
| | NO: | | NO: 230 | |
| | 119 | | | |
| D: 8 | SEQ ID | AAUGCAGAAGAUGUAGAUUGA | SEQ ID | UCAAUCUACAUCUUCUGCAUUUG |
| | NO: | | NO: 231 | |
| | 120 | | | |
| D: 9 | SEQ ID | GCUUUGUUCGAAAGAAUGGUA | SEQ ID | UACCAUUCUUUCGAACAAAGCCU |
| | NO: | | NO: 232 | |
| | 121 | | | |
| D: 10 | SEQ ID | GACUUGAGUAAAUAUAUCACA | SEQ ID | UGUGAUAUAUUUACUCAAGUCAA |
| | NO: | | NO: 233 | |
| | 122 | | | |
| D: 11 | SEQ ID | AGAUGAGAUCAAGAAUGACAA | SEQ ID | UUGUCAUUCUUGAUCUCAUCUAU |
| | NO: | | NO: 234 | |
| | 123 | | | |
| D: 12 | SEQ ID | UUGAGUAAAUAUAUCACCACA | SEQ ID | UGUGGUGAUAUAUUUACUCAAGU |
| | NO: | | NO: 235 | |
| | 124 | | | |
| D: 13 | SEQ ID | AAUAGAUGAGAUCAAGAAUGA | SEQ ID | UCAUUCUUGAUCUCAUCUAUUUU |
| | NO: | | NO: 236 | |
| | 125 | | | |
| D: 14 | SEQ ID | CUUUGUUCGAAAGAAUGGUGA | SEQ ID | UCACCAUUCUUUCGAACAAAGCC |
| | NO: | | NO: 237 | |
| | 126 | | | |
| D: 15 | SEQ ID | UGUGAUGAAGGACAUGGCUUA | SEQ ID | UAAGCCAUGUCCUUCAUCACACA |
| | NO: | | NO: 238 | |
| | 127 | | | |
| D: 16 | SEQ ID | UGACUUGAGUAAAUAUAUCAA | SEQ ID | UUGAUAUAUUUACUCAAGUCAAC |
| | NO: | | NO: 239 | |
| | 128 | | | |
| D: 17 | SEQ ID | GAAAGUUCAACUGCUUCGUAA | SEQ ID | UUACGAAGCAGUUGAACUUUCUG |
| | NO: | | NO: 240 | |
| | 129 | | | |
| D: 18 | SEQ ID | GCAGAAGAUGUAGAUUGUGUA | SEQ ID | UACACAAUCUACAUCUUCUGCAU |
| | NO: | | NO: 241 | |
| | 130 | | | |
| D: 19 | SEQ ID | UGUGAACACUGUGACCCUUGA | SEQ ID | UCAAGGGUCACAGUGUUCACAUA |
| | NO: | | NO: 242 | |
| | 131 | | | |
| D: 20 | SEQ ID | GACACUAAGUCAAGUUAAAGA | SEQ ID | UCUUUAACUUGACUUAGUGUCAU |
| | NO: | | NO: 243 | |
| | 132 | | | |
| D: 21 | SEQ ID | GAAUACCAAGUGCAGAUGUAA | SEQ ID | UUACAUCUGCACUUGGUAUUCUG |
| | NO: | | NO: 244 | |
| | 133 | | | |
| D: 22 | SEQ ID | GACAAUGUCCAAGACACAGCA | SEQ ID | UGCUGUGUCUUGGACAUUGUCAU |
| | NO: | | NO: 245 | |
| | 134 | | | |
| D: 23 | SEQ ID | AUGAAGGACAUGGCUUAGAAA | SEQ ID | UUUCUAAGCCAUGUCCUUCAUCA |
| | NO: | | NO: 246 | |
| | 135 | | | |
| D: 24 | SEQ ID | UUCCAAAUGCAGAAGAUGUAA | SEQ ID | UUACAUCUUCUGCAUUUGGAAGA |
| | NO: | | NO: 247 | |
| | 136 | | | |
| D: 25 | SEQ ID | GAGAUCAAGAAUGACAAUGUA | SEQ ID | UACAUUGUCAUUCUUGAUCUCAU |
| | NO: | | NO: 248 | |
| | 137 | | | |
| D: 26 | SEQ ID | CAAGUGCAGAUGUAAACCAAA | SEQ ID | UUUGGUUUACAUCUGCACUUGGU |
| | NO: | | NO: 249 | |
| | 138 | | | |
| D: 27 | SEQ ID | UUUGUUCGAAAGAAUGGUGUA | SEQ ID | UACACCAUUCUUUCGAACAAAGC |
| | NO: | | NO: 250 | |
| | 139 | | | |
| D: 28 | SEQ ID | CAGAAGAUGUAGAUUGUGUGA | SEQ ID | UCACACAAUCUACAUCUUCUGCA |
| | NO: | | NO: 251 | |
| | 140 | | | |
| D: 29 | SEQ ID | UCUUUGUACUCUUGCAGAGAA | SEQ ID | UUCUCUGCAAGAGUACAAAGAUU |
| | NO: | | NO: 252 | |
| | 141 | | | |
| D: 30 | SEQ ID | CACUGUGACCCUUGCACCAAA | SEQ ID | UUUGGUGCAAGGGUCACAGUGUU |
| | NO: | | NO: 253 | |
| | 142 | | | |
| D: 31 | SEQ ID | UCCAAAUGCAGAAGAUGUAGA | SEQ ID | UCUACAUCUUCUGCAUUUGGAAG |
| | NO: | | NO: 254 | |
| | 143 | | | |
| D: 32 | SEQ ID | AAUGUCCAAGACACAGCAGAA | SEQ ID | UUCUGCUGUGUCUUGGACAUUGU |
| | NO: | | NO: 255 | |
| | 144 | | | |
| D: 33 | SEQ ID | GCCAAGAAGGGAAGGAGUACA | SEQ ID | UGUACUCCUUCCCUUCUUGGCAG |
| | NO: | | NO: 256 | |
| | 145 | | | |
| D: 34 | SEQ ID | CUUGAGUAAAUAUAUCACCAA | SEQ ID | UUGGUGAUAUAUUUACUCAAGUC |
| | NO: | | NO: 257 | |
| | 146 | | | |
| D: 35 | SEQ ID | UAGAUGAGAUCAAGAAUGACA | SEQ ID | UGUCAUUCUUGAUCUCAUCUAUU |
| | NO: | | NO: 258 | |
| | 147 | | | |
| D: 36 | SEQ ID | UUGACUUGAGUAAAUAUAUCA | SEQ ID | UGAUAUAUUUACUCAAGUCAACA |
| | NO: | | NO: 259 | |
| | 148 | | | |
| D: 37 | SEQ ID | AGUAAAUAUAUCACCACUAUA | SEQ ID | UAUAGUGGUGAUAUAUUUACUCA |
| | NO: | | NO: 260 | |
| | 149 | | | |
| D: 38 | SEQ ID | UACCAAGUGCAGAUGUAAACA | SEQ ID | UGUUUACAUCUGCACUUGGUAUU |
| | NO: | | NO: 261 | |
| | 150 | | | |
| D: 39 | SEQ ID | CUGUAUGUGAACACUGUGACA | SEQ ID | UGUCACAGUGUUCACAUACAGUA |
| | NO: | | NO: 262 | |
| | 151 | | | |
| D: 40 | SEQ ID | GAUCAAGAAUGACAAUGUCCA | SEQ ID | UGGACAUUGUCAUUCUUGAUCUC |
| | NO: | | NO: 263 | |
| | 152 | | | |
| D: 41 | SEQ ID | AUGACACUAAGUCAAGUUAAA | SEQ ID | UUUAACUUGACUUAGUGUCAUGA |
| | NO: | | NO: 264 | |
| | 153 | | | |
| D: 42 | SEQ ID | AAAUAGAUGAGAUCAAGAAUA | SEQ ID | UAUUCUUGAUCUCAUCUAUUUUG |
| | NO: | | NO: 265 | |
| | 154 | | | |
| D: 43 | SEQ ID | GUAUGUGAACACUGUGACCCA | SEQ ID | UGGGUCACAGUGUUCACAUACAG |
| | NO: | | NO: 266 | |
| | 155 | | | |
| D: 44 | SEQ ID | AAUACCAAGUGCAGAUGUAAA | SEQ ID | UUUACAUCUGCACUUGGUAUUCU |
| | NO: | | NO: 267 | |
| | 156 | | | |
| D: 45 | SEQ ID | UGAGAUCAAGAAUGACAAUGA | SEQ ID | UCAUUGUCAUUCUUGAUCUCAUC |
| | NO: | | NO: 268 | |
| | 157 | | | |
| D: 46 | SEQ ID | CUUUGUACUCUUGCAGAGAAA | SEQ ID | UUUCUCUGCAAGAGUACAAAGAU |
| | NO: | | NO: 269 | |
| | 158 | | | |
| D: 47 | SEQ ID | UGAAGCCAAAAUAGAUGAGAA | SEQ ID | UUCUCAUCUAUUUUGGCUUCAUU |
| | NO: | | NO: 270 | |
| | 159 | | | |
| D: 48 | SEQ ID | CAUCCUCAAGGACAUUACUAA | SEQ ID | UUAGUAAUGUCCUUGAGGAUGAU |
| | NO: | | NO: 271 | |
| | 160 | | | |
| D: 49 | SEQ ID | UAUGUGAACACUGUGACCCUA | SEQ ID | UAGGGUCACAGUGUUCACAUACA |
| | NO: | | NO: 272 | |
| | 161 | | | |
| D: 50 | SEQ ID | GUGUUAAUGCCCAAGUGACUA | SEQ ID | UAGUCACUUGGGCAUUAACACUU |
| | NO: | | NO: 273 | |
| | 162 | | | |
| D: 51 | SEQ ID | UUCAACUGCUUCGUAAUUGGA | SEQ ID | UCCAAUUACGAAGCAGUUGAACU |
| | NO: | | NO: 274 | |
| | 163 | | | |
| D: 52 | SEQ ID | UGCAGAAGAUGUAGAUUGUGA | SEQ ID | UCACAAUCUACAUCUUCUGCAUU |
| | NO: | | NO: 275 | |
| | 164 | | | |
| D: 53 | SEQ ID | GAGUAAAUAUAUCACCACUAA | SEQ ID | UUAGUGGUGAUAUAUUUACUCAA |
| | NO: | | NO: 276 | |
| | 165 | | | |
| D: 54 | SEQ ID | CAAGGAAUGCACACUCACCAA | SEQ ID | UUGGUGAGUGUGCAUUCCUUGAU |
| | NO: | | NO: 277 | |
| | 166 | | | |
| D: 55 | SEQ ID | CAGGUGAAAGGAAAGCUAGGA | SEQ ID | UCCUAGCUUUCCUUUCACCUGGA |
| | NO: | | NO: 278 | |
| | 167 | | | |
| D: 56 | SEQ ID | AAUGACAAUGUCCAAGACACA | SEQ ID | UGUGUCUUGGACAUUGUCAUUCU |
| | NO: | | NO: 279 | |
| | 168 | | | |
| D: 57 | SEQ ID | GGAAUGCACACUCACCAGCAA | SEQ ID | UUGCUGGUGAGUGUGCAUUCCUU |
| | NO: | | NO: 280 | |
| | 169 | | | |
| D: 58 | SEQ ID | CCUGUCCUCCAGGUGAAAGGA | SEQ ID | UCCUUUCACCUGGAGGACAGGGC |
| | NO: | | NO: 281 | |
| | 170 | | | |
| D: 59 | SEQ ID | AUGCAGAAGAUGUAGAUUGUA | SEQ ID | UACAAUCUACAUCUUCUGCAUUU |
| | NO: | | NO: 282 | |
| | 171 | | | |
| D: 60 | SEQ ID | UUGUGUGAUGAAGGACAUGGA | SEQ ID | UCCAUGUCCUUCAUCACACAAUC |
| | NO: | | NO: 283 | |
| | 172 | | | |
| D: 61 | SEQ ID | AAAGUUCAACUGCUUCGUAAA | SEQ ID | UUUACGAAGCAGUUGAACUUUCU |
| | NO: | | NO: 284 | |
| | 173 | | | |
| D: 62 | SEQ ID | UGUUAAUGCCCAAGUGACUGA | SEQ ID | UCAGUCACUUGGGCAUUAACACU |
| | NO: | | NO: 285 | |
| | 174 | | | |
| D: 63 | SEQ ID | GAUGUUGACUUGAGUAAAUAA | SEQ ID | UUAUUUACUCAAGUCAACAUCAG |
| | NO: | | NO: 286 | |
| | 175 | | | |
| D: 64 | SEQ ID | GAAGCCAAAAUAGAUGAGAUA | SEQ ID | UAUCUCAUCUAUUUUGGCUUCAU |
| | NO: | | NO: 287 | |
| | 176 | | | |
| D: 65 | SEQ ID | GUUCAACUGCUUCGUAAUUGA | SEQ ID | UCAAUUACGAAGCAGUUGAACUU |
| | NO: | | NO: 288 | |
| | 177 | | | |
| D: 66 | SEQ ID | GUACUCUUGCAGAGAAAAUUA | SEQ ID | UAAUUUUCUCUGCAAGAGUACAA |
| | NO: | | NO: 289 | |
| | 178 | | | |
| D: 67 | SEQ ID | CUCAAGGACAUUACUAGUGAA | SEQ ID | UUCACUAGUAAUGUCCUUGAGGA |
| | NO: | | NO: 290 | |
| | 179 | | | |
| D: 68 | SEQ ID | AGGGAAGGAGUACACAGACAA | SEQ ID | UUGUCUGUGUACUCCUUCCCUUC |
| | NO: | | NO: 291 | |
| | 180 | | | |
| D: 69 | SEQ ID | UCAUGACACUAAGUCAAGUUA | SEQ ID | UAACUUGACUUAGUGUCAUGACU |
| | NO: | | NO: 292 | |
| | 181 | | | |
| D: 70 | SEQ ID | CCUCAAGGACAUUACUAGUGA | SEQ ID | UCACUAGUAAUGUCCUUGAGGAU |
| | NO: | | NO: 293 | |
| | 182 | | | |
| D: 71 | SEQ ID | GGCUUUGUUCGAAAGAAUGGA | SEQ ID | UCCAUUCUUUCGAACAAAGCCUU |
| | NO: | | NO: 294 | |
| | 183 | | | |
| D: 72 | SEQ ID | CCAGACUGCGUGCCCUGCCAA | SEQ ID | UUGGCAGGGCACGCAGUCUGGUU |
| | NO: | | NO: 295 | |
| | 184 | | | |
| D: 73 | SEQ ID | AGCCAAAAUAGAUGAGAUCAA | SEQ ID | UUGAUCUCAUCUAUUUUGGCUUC |
| | NO: | | NO: 296 | |
| | 185 | | | |
| D: 74 | SEQ ID | AGAAGGGAAGGAGUACACAGA | SEQ ID | UCUGUGUACUCCUUCCCUUCUUG |
| | NO: | | NO: 297 | |
| | 186 | | | |
| D: 75 | SEQ ID | AGAUUGUGUGAUGAAGGACAA | SEQ ID | UUGUCCUUCAUCACACAAUCUAC |
| | NO: | | NO: 298 | |
| | 187 | | | |
| D: 76 | SEQ ID | UUUCACUUCGGAGGAUUGCUA | SEQ ID | UAGCAAUCCUCCGAAGUGAAAGA |
| | NO: | | NO: 299 | |
| | 188 | | | |
| D: 77 | SEQ ID | AAGGAAAGCUAGGGACUGCAA | SEQ ID | UUGCAGUCCCUAGCUUUCCUUUC |
| | NO: | | NO: 300 | |
| | 189 | | | |
| D: 78 | SEQ ID | GACAUGGCUUAGAAGUGGAAA | SEQ ID | UUUCCACUUCUAAGCCAUGUCCU |
| | NO: | | NO: 301 | |
| | 190 | | | |
| D: 79 | SEQ ID | UGUACUCUUGCAGAGAAAAUA | SEQ ID | UAUUUUCUCUGCAAGAGUACAAA |
| | NO: | | NO: 302 | |
| | 191 | | | |
| D: 80 | SEQ ID | AUGAACCAGACUGCGUGCCCA | SEQ ID | UGGGCACGCAGUCUGGUUCAUCC |
| | NO: | | NO: 303 | |
| | 192 | | | |
| D: 81 | SEQ ID | CAUGACACUAAGUCAAGUUAA | SEQ ID | UUAACUUGACUUAGUGUCAUGAC |
| | NO: | | NO: 304 | |
| | 193 | | | |
| D: 82 | SEQ ID | UUGUUCGAAAGAAUGGUGUCA | SEQ ID | UGACACCAUUCUUUCGAACAAAG |
| | NO: | | NO: 305 | |
| | 194 | | | |
| D: 83 | SEQ ID | ACACUGUGACCCUUGCACCAA | SEQ ID | UUGGUGCAAGGGUCACAGUGUUC |
| | NO: | | NO: 306 | |
| | 195 | | | |
| D: 84 | SEQ ID | GGAAAUAAACUGCACCCGGAA | SEQ ID | UUCCGGGUGCAGUUUAUUUCCAC |
| | NO: | | NO: 307 | |
| | 196 | | | |
| D: 85 | SEQ ID | AAGUGUUAAUGCCCAAGUGAA | SEQ ID | UUCACUUGGGCAUUAACACUUUU |
| | NO: | | NO: 308 | |
| | 197 | | | |
| D: 86 | SEQ ID | AGUUCAACUGCUUCGUAAUUA | SEQ ID | UAAUUACGAAGCAGUUGAACUUU |
| | NO: | | NO: 309 | |
| | 198 | | | |
| D: 87 | SEQ ID | CACUUCGGAGGAUUGCUCAAA | SEQ ID | UUUGAGCAAUCCUCCGAAGUGAA |
| | NO: | | NO: 310 | |
| | 199 | | | |
| D: 88 | SEQ ID | AUGAAGCCAAAAUAGAUGAGA | SEQ ID | UCUCAUCUAUUUUGGCUUCAUUG |
| | NO: | | NO: 311 | |
| | 200 | | | |
| D: 89 | SEQ ID | ACUUCGGAGGAUUGCUCAACA | SEQ ID | UGUUGAGCAAUCCUCCGAAGUGA |
| | NO: | | NO: 312 | |
| | 201 | | | |
| D: 90 | SEQ ID | AGGUGAAAGGAAAGCUAGGGA | SEQ ID | UCCCUAGCUUUCCUUUCACCUGG |
| | NO: | | NO: 313 | |
| | 202 | | | |
| D: 91 | SEQ ID | AAGGGAAGGAGUACACAGACA | SEQ ID | UGUCUGUGUACUCCUUCCCUUCU |
| | NO: | | NO: 314 | |
| | 203 | | | |
| D: 92 | SEQ ID | AGUGGAAAUAAACUGCACCCA | SEQ ID | UGGGUGCAGUUUAUUUCCACUUC |
| | NO: | | NO: 315 | |
| | 204 | | | |
| D: 93 | SEQ ID | AUGACAAUGUCCAAGACACAA | SEQ ID | UUGUGUCUUGGACAUUGUCAUUC |
| | NO: | | NO: 316 | |
| | 205 | | | |
| D: 94 | SEQ ID | GAUUGUGUGAUGAAGGACAUA | SEQ ID | UAUGUCCUUCAUCACACAAUCUA |
| | NO: | | NO: 317 | |
| | 206 | | | |
| D: 95 | SEQ ID | GGGGAUGAACCAGACUGCGUA | SEQ ID | UACGCAGUCUGGUUCAUCCCCAU |
| | NO | | NO: 318 | |
| | 207 | | | |
| D: 96 | SEQ ID | GUUAAUGCCCAAGUGACUGAA | SEQ ID | UUCAGUCACUUGGGCAUUAACAC |
| | NO: | | NO: 319 | |
| | 208 | | | |
| D: 97 | SEQ ID | ACCAGACUGCGUGCCCUGCCA | SEQ ID | UGGCAGGGCACGCAGUCUGGUUC |
| | NO: | | NO: 320 | |
| | 209 | | | |
| D: 98 | SEQ ID | AACACUGUGACCCUUGCACCA | SEQ ID | UGGUGCAAGGGUCACAGUGUUCA |
| | NO: | | NO: 321 | |
| | 210 | | | |
| D: 99 | SEQ ID | GGAAGGAGUACACAGACAAAA | SEQ ID | UUUUGUCUGUGUACUCCUUCCCU |
| | NO: | | NO: 322 | |
| | 211 | | | |
| D: 100 | SEQ ID | GGGAUGAACCAGACUGCGUGA | SEQ ID | UCACGCAGUCUGGUUCAUCCCCA |
| | NO: | | NO: 323 | |
| | 212 | | | |
| D: 101 | SEQ ID | CUGCGUGCCCUGCCAAGAAGA | SEQ ID | UCUUCUUGGCAGGGCACGCAGUC |
| | NO: | | NO: 324 | |
| | 213 | | | |
| D: 102 | SEQ ID | UGAACACUGUGACCCUUGCAA | SEQ ID | UUGCAAGGGUCACAGUGUUCACA |
| | NO: | | NO: 325 | |
| | 214 | | | |
| D: 103 | SEQ ID | AGGAUUGCUCAACAACCAUGA | SEQ ID | UCAUGGUUGUUGAGCAAUCCUCC |
| | NO: | | NO: 326 | |
| | 215 | | | |
| D: 104 | SEQ ID | UGACACUAAGUCAAGUUAAAA | SEQ ID | UUUUAACUUGACUUAGUGUCAUG |
| | NO: | | NO: 327 | |
| | 216 | | | |
| D: 105 | SEQ ID | UGAAAGGAAAGCUAGGGACUA | SEQ ID | UAGUCCCUAGCUUUCCUUUCACC |
| | NO: | | NO: 328 | |
| | 217 | | | |
| D: 106 | SEQ ID | ACAAUGUCCAAGACACAGCAA | SEQ ID | UUGCUGUGUCUUGGACAUUGUCA |
| | NO: | | NO: 329 | |
| | 218 | | | |
| D: 107 | SEQ ID | GUGAAAGGAAAGCUAGGGACA | SEQ ID | UGUCCCUAGCUUUCCUUUCACCU |
| | NO: | | NO: 330 | |
| | 219 | | | |
| D: 108 | SEQ ID | AGCUCUUUCACUUCGGAGGAA | SEQ ID | UUCCUCCGAAGUGAAAGAGCUUC |
| | NO: | | NO: 331 | |
| | 220 | | | |
| D: 109 | SEQ ID | UUCUUCCAAAUGCAGAAGAUA | SEQ ID | UAUCUUCUGCAUUUGGAAGAAAA |
| | NO: | | NO: 332 | |
| | 221 | | | |
| D: 110 | SEQ ID | GGAGGAUUGCUCAACAACCAA | SEQ ID | UUGGUUGUUGAGCAAUCCUCCGA |
| | NO | | NO: 333 | |
| | 222 | | | |
| D: 111 | SEQ ID | UGCCAAGAAGGGAAGGAGUAA | SEQ ID | UUACUCCUUCCCUUCUUGGCAGG |
| | NO: | | NO: 334 | |
| | 223 | | | |
| D: 112 | SEQ ID | GAAAGUUCA-(AP)- | SEQ ID | UUACGAAGCAGUUGAACUUUCUG |
| | NO: | CUGCUUCGUAA | NO: 240 | |
| | 335 | | | |
| D: 113 | SEQ ID | GUUGACUUG-(AP)- | SEQ ID | UAUAUAUUUACUCAAGUCAACAU |
| | NO: | GUAAAUAUAUA | NO: 227 | |
| | 336 | | | |
| D: 114 | SEQ ID | GUUGACUUGAGUAAAUAUAUA | SEQ ID | UAUAU-GNA (A)- |
| | NO: | | NO: 337 | UUUACUCAAGUCAACAU |
| | 116 | | | |
| D: 115 | SEQ ID | GUUGACUUGAGUAAAUAUAUA | SEQ ID | UAUAUA-GNA (U)-UUACUCAAGUCAACAU |
| | NO: | | NO: 338 | |
| | 116 | | | |
| D:236 | SEQ ID | AGAAAGUUCAACUGCUUCGUA | SEQ ID | UACGAAGCAGUUGAACUUUCUGU |
| | NO: | | NO: 577 | |
| | 576 | | | |

GNA indicates a glycol nucleic acid nucleotide (structure shown in Table 3B); (AP) means an apurinic/apyrimidinic residue, also called an abasic residue (structure shown in Table 3B).

TABLE 3B
Structures of GNA and abasic residue

| | |
|---|---|
| GNA | 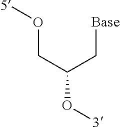 |
| Abasic | 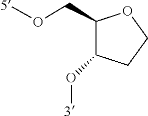 |

### Example 4

### In Vitro Knockdown of hFAS in HepG2 Cells

The RNAi agents in Tables 4A and 4B were tested in HepG2 cells. Reverse transfection was carried out by adding 24.7 μl of Opti-MEM plus 0.3 μl of Lipofectamine RNAiMAX per well to 25 μl of each 4× human FAS-GalNAc siRNA to an individual well in a 96-well collagen I-coated plate. The mixture was incubated at room temperature for 20 minutes and then fifty μl of Growth Media containing HepG2 cells at 300,000 cells/ml were added to the human FAS-GalNAc siRNA/RNAiMAX mixture. Final concentration of the siRNAs as with the above was 500 nM for a single concentration screen. Cells were incubated for 24-48 hours before RNA isolation with Quick-RNA 96 Kit. RNA was then stored at −80° C. or subject to cDNA synthesis. Briefly, cDNA was synthesized from the purified RNA using Fast Advanced RT Master Mix (Invitrogen). A master mix of 5 μl 2× Fast Advanced RT Buffer and 0.5 μl 20× Fast Advanced RT Enzyme Mix per reaction was prepared. 5.5 μl master mix and 4.5 μl RNA were mixed for a final volume of 10 μl. cDNA was generated using a ProFlex PCR System (Life Technologies) through the following steps: 37° C. for 30 minutes, 95° C. for 5 minutes, and 4° C. hold.

Two μl of cDNA were added to a master mix containing 2.5 μl of H2O, 0.5 μl 20× TaqMan Gene Expression Assay Buffer (Life Technologies) and 5 μl 2× TaqMan Universal PCR Master Mix (Life Technologies). A QuantStudio 7 Flex Real-Time PCR System (Life Technologies) was used to complete the following PCR cycles: 50° C. for 2 minutes, 95° C. for 10 minutes, 40 cycles of 95° C. for 15 seconds and 60° C. for 1 minute. TaqMan Gene Expression Assays were performed. Data analysis uses the ddCt method.

Select siRNAs from each assay were used for determining an IC50, using 1:3 serial dilution to final concentrations of 200, 67, 22, 7.41, 2.47, 0.82, and 0.27 nM FAS-GalNAc RNAi agent for concentration response curves. IC50 values were calculated using a 4-parameter fit model using XLFit.

Data is shown in Table 5.

### Example 5

### In Vivo Knockdown in hFAS-AAV Treated Mice with the FAS RNAi Agents Herein

Mice were administered AAV vector for expressing human FAS (1×1011 GC/mouse) via retroorbital injection after anesthetization via isoflurane. 100 ul of AAV (in PBS) is injected into the venous sinus and mice were monitored for recovery in cage. Two weeks following AAV administration, mice were administered a set of siRNA agents of Table 4A and 4B, as indicated in Table 6A and 6B, subcutaneously, except that all siRNA agents were lacking the phosphate addition on the 5′ end of the antisense strand for administration to mice.

Mice were sacrificed, and serum and livers (in RNAlater Stabilization Solution, Ambion) were collected. Total liver RNA was isolated, purified, and subject to QRT-PCR as described above.

Results showed the gene expression of human FAS target gene normalized to mouse Rplp0 (Life Technologies, part #: Mm01974474_gH), and represented as the relative knockdown of human FAS mRNA expression compared to vehicle-treated control animals. Knockdown results at 2 weeks post treatment at 5 mg/kg (mpk), or 10 weeks at 1 mg/kg, 3 mg/kg and 5 mg/kg doses are shown for the RNAi agents indicated in Tables 6A and 6B.

Some RNAi agents tested for gene expression knockdown in vivo were further tested for protein knock-down per Example 6.

TABLE 4A
FAS-GalNAc RNAi agents, modified sense and antisense strands

| Duplex | SEQ ID NO: | Modified Sequence |
|---|---|---|
| D: 112 | SEQ ID NO: 339 | mG*mA*mUmGmAmAfGmGfAfCfAmUmGmGmCmUmUmAmG*mA*mAP |
| | SEQ ID NO: 340 | PmU*fU*mCmUmAfAmGmCmCmAmUmGmUfCmCfUmUmCmAmUmC*mA*mC |
| D: 113 | SEQ ID NO: 341 | mU*mA*mAmAmCmCfAmAfAfCfUmUmUmUmUmUmUmGmU*mA*mAP |
| | SEQ ID NO: 342 | PmU*fU*mAmCmAfAmAmAmAmAmAmGmUfUmUfGmGmUmUmUmA*mC*mA |
| D: 114 | SEQ ID NO: 343 | mG*mA*mAmGmAmUfGmUfAfGfAmUmUmGmUmGmUmGmA*mU*mAP |
| | SEQ ID NO: 344 | PmU*fA*mUmCmAfCmAmCmAmAmUmCmUfAmCfAmUmCmUmUmC*mU*mG |
| D: 115 | SEQ ID NO: 345 | mG*mU*mUmGmAmCfUmUfGfAfGmUmAmAmAmUmAmUmA*mU*mAP |
| | SEQ ID NO: 346 | PmU*fA*mUmAmUfAmUmUmUmAmCmUmCfAmAfGmUmCmAmAmC*mA*mU |
| D: 116 | SEQ ID NO: 347 | mA*mUmCmAmAmGfAmAfUfGfAmCmAmAmUmGmUmCmC*mA*mAP |
| | SEQ ID NO: 348 | PmU*fU*mGmGmAfCmAmUmUmGmUmCmAfUmUfCmUmUmGmAmU*mC*mU |
| D: 117 | SEQ ID NO: 349 | mU*mG*mUmUmGmAfCmUfUfGfAmGmUmAmAmAmUmAmU*mA*mAP |
| | SEQ ID NO: 350 | PmU*fU*mAmUmAfUmUmUmAmCmUmCmAfAmGfUmCmAmAmCmA*mU*mC |
| D: 118 | SEQ ID NO: 351 | mG*mC*mCmAmAmAfAmUfAfGfAmUmGmAmGmAmUmCmA*mA*mAP |
| | SEQ ID NO: 352 | PmU*fUmUmGmAfUmCmUmCmAmUmCmUfAmUfUmUmUmGmGmC*mU*mU |
| D: 119 | SEQ ID NO: 353 | mA*mA*mUmGmCmAfGmAfAfGfAmUmGmUmAmGmAmUmU*mG*mAP |
| | SEQ ID NO: 354 | PmU*fC*mAmAmUfCmUmAmCmAmUmCmUfUmCfUmGmCmAmUmU*mU*mG |
| D: 120 | SEQ ID NO: 355 | mG*mC*mUmUmUmGfUmUfCfGfAmAmAmGmAmAmUmGmG*mU*mAP |
| | SEQ ID NO: 356 | PmU*fA*mCmCmAfUmUmCmUmUmUmCmGfAmAfCmAmAmAmGmC*mC*mU |
| D: 121 | SEQ ID NO: 357 | mG*mA*mCmUmUmGfAmGfUfAfAmAmUmAmUmAmUmCmA*mC*mAP |
| | SEQ ID NO: 358 | PmU*G*mUmGmAfUmAmUmAmUmUmUmAfCmUfCmAmAmGmUmC*mA*mA |
| D: 122 | SEQ ID NO: 359 | mA*mG*mAmUmGmAfGmAfUfCfAmAmGmAmAmUmGmAmC*mA*mAP |
| | SEQ ID NO: 360 | PmU*U*mGmUmCfAmUmUmCmUmUmGmAfUmCfUmCmAmUmCmU*mA*mU |
| D: 123 | SEQ ID NO: 361 | mU*mUmGmAmGmUfAmAfAfUfAmUmAmUmCmAmCmCmA*mC*mAP |
| | SEQ ID NO: 362 | PmU*fG*mUmGmGfUmGmAmUmAmUmAmUfUmUfAmCmUmCmAmA*mG*mU |
| D: 124 | SEQ ID NO: 363 | mA*mA*mUmAmGmAfUmGfAfGfAmUmCmAmAmGmAmAmU*mG*mAP |
| | SEQ ID NO: 364 | PmU*fC*mAmUmUfCmUmUmGmAmUmCmUfCmAfUmCmUmAmUmU*mU*mU |
| D: 125 | SEQ ID NO: 365 | mC*mUmUmUmGmUfUmCfGfAfAmAmGmAmAmUmGmGmU*mG*mAP |
| | SEQ ID NO: 366 | PmU*fC*mAmCmCfAmUmUmCmUmUmUmCfGmAfAmCmAmAmAmG*mC*mC |
| D: 126 | SEQ ID NO: 367 | mU*mG*mUmGmAmUfGmAfAfGfGmAmCmAmUmGmGmCmU*mU*mAP |
| | SEQ ID NO: 368 | PmU*fA*mAmGmCfCmAmUmGmUmCmCmUfUmCfAmUmCmAmCmA*mC*mA |
| D: 127 | SEQ ID NO: 369 | mU*mG*mAmCmUmUfGmAfGfUfAmAmAmUmAmUmAmUmC*mA*mAP |
| | SEQ ID NO: 370 | PmU*fU*mGmAmUfAmUmAmUmUmUmAmCfUmCfAmAmGmUmCmA*mA*mC |
| D: 128 | SEQ ID NO: 371 | mG*mA*mAmAmGmUfUmCfAfAfCmUmGmCmUmUmCmGmU*mA*mAP |
| | SEQ ID NO: 372 | PmU*fU*mAmCmGfAmAmGmCmAmGmUmUfGmAfAmCmUmUmUmC*mU*mG |
| D: 129 | SEQ ID NO: 373 | mG*mC*mAmGmAmAfGmAfUfGfUmAmGmAmUmUmGmUmG*mU*mAP |
| | SEQ ID NO: 374 | PmU*fA*mCmAmCfAmAmUmCmUmAmCmAfUmCfUmUmCmUmGmC*mA*mU |
| D: 130 | SEQ ID NO: 375 | mU*mG*mUmGmAmAfCmAfCfUfGmUmGmAmCmCmCmUmU*mG*mAP |
| | SEQ ID NO: 376 | PmU*fC*mAmAmGfGmGmUmCmAmCmAmGfUmGfUmUmCmAmCmA*mU*mA |
| D: 131 | SEQ ID NO: 377 | mG*mA*mCmAmCmUfAmAfGfUfCmAmAmGmUmUmAmAmA*mG*mAP |
| | SEQ ID NO: 378 | PmU*fC*mUmUmUfAmAmCmUmUmGmAmCfUmUfAmGmUmGmUmC*mA*mU |
| D: 132 | SEQ ID NO: 379 | mG*mA*mAmUmAmCfCmAfAfGfUmGmCmAmGmAmUmGmU*mA*mAP |
| | SEQ ID NO: 380 | PmU*fU*mAmCmAfUmCmUmGmCmAmCmUfUmGfGmUmAmUmUmC*mU*mG |
| D: 133 | SEQ ID NO: 381 | mG*mA*mCmAmAmUfGmUfCfCfAmAmGmAmCmAmCmAmG*mC*mAP |
| | SEQ ID NO: 382 | PmU*fG*mCmUmGfUmGmUmCmUmUmGmGfAmCfAmUmUmGmUmC*mA*mU |
| D: 134 | SEQ ID NO: 383 | mA*mU*mGmAmAmGfGmAfCfAfUmGmGmCmUmUmAmGmA*mA*mAP |
| | SEQ ID NO: 384 | PmU*fU*mUmCmUfAmAmGmCmCmAmUmGfUmCfCmUmUmCmAmU*mC*mA |
| D: 135 | SEQ ID NO: 385 | mU*mU*mCmCmAmAfAmUfGfCfAmGmAmAmGmAmUmGmU*mA*mAP |
| | SEQ ID NO: 386 | PmU*fU*mAmCmAfUmCmUmUmCmUmGmCfAmUfUmUmGmGmAmA*mG*mA |
| D: 136 | SEQ ID NO: 387 | mG*mA*mGmAmUmCfAmAfGfAfAmUmGmAmCmAmAmUmG*mU*mAP |
| | SEQ ID NO: 388 | PmU*FA*mCmAmUfUmGmUmCmAmUmUmCfUmUfGmAmUmCmUmC*mA*mU |
| D: 137 | SEQ ID NO: 389 | mC*mA*mAmGmUmGfCmAfGfAfUmGmUmAmAmAmCmCmA*mA*mAP |
| | SEQ ID NO: 390 | PmU*fUmUmGmGfUmUmUmAmCmAmUmCfUmGfCmAmCmUmUmG*mG*mU |
| D: 138 | SEQ ID NO: 391 | mU*mU*mUmGmUmUfCmGfAfAfAmGmAmAmUmGmGmUmG*mU*mAP |
| | SEQ ID NO: 392 | PmU*fA*mCmAmCfCmAmUmUmCmUmUmUfCmGfAmAmCmAmAmA*mG*mC |
| D: 139 | SEQ ID NO: 393 | mC*mA*mGmAmAmGfAmUfGfUfAmGmAmUmUmGmUmGmU*mG*mAP |
| | SEQ ID NO: 394 | PmU*fC*mAmCmAfCmAmAmUmCmUmAmCfAmUfCmUmUmCmUmG*mC*mA |
| D: 140 | SEQ ID NO: 395 | mU*mC*mUmUmUmGfUmAfCfUfCmUmUmGmCmAmGmAmG*mA*mAP |
| | SEQ ID NO: 396 | PmU*fU*mCmUmCfUmGmCmAmAmGmAmGfUmAfCmAmAmAmGmA*mU*mU |
| D: 141 | SEQ ID NO: 397 | mC *mA*mCmUmGmUfGmAfCfCfCmUmUmGmCmAmCmCmA*mA*mAP |
| | SEQ ID NO: 398 | PmU*fU*mUmGmGfUmGmCmAmAmGmGmGfUmCfAmCmAmGmUmG*mU*mU |
| D: 142 | SEQ ID NO: 399 | mU*mC*mCmAmAmAfUmGfCfAfGmAmAmGmAmUmGmUmA*mG*mAP |
| | SEQ ID NO: 400 | PmU*C*mUmAmCfAmUmCmUmUmCmUmGfCmAfUmUmUmGmGmA*mA*mG |
| D: 143 | SEQ ID NO: 401 | mA*mA*mUmGmUmCfCmAfAfGfAmCmAmCmAmGmCmAmG*mA*mAP |
| | SEQ ID NO: 402 | PmU*fU*mCmUmGfCmUmGmUmGmUmCmUfUmGfGmAmCmAmUmU*mG*mU |
| D: 144 | SEQ ID NO: 403 | mG*mC*mCmAmAmGfAmAfGfGfGmAmAmGmGmAmGmUmA*mC*mAP |
| | SEQ ID NO: 404 | PmU*fG*mUmAmCfUmCmCmUmUmCmCmCfUmUfCmUmUmGmGmC*mA*mG |
| D: 145 | SEQ ID NO: 405 | mC*mUmUmGmAmGfUmAfAfAfUmAmUmAmUmCmAmCmC*mA*mAP |
| | SEQ ID NO: 406 | PmU*fU*mGmGmUfGmAmUmAmUmAmUmUfUmAfCmUmCmAmAmG*mU*mC |
| D: 146 | SEQ ID NO: 407 | mU*mA*mGmAmUmGfAmGfAfUfCmAmAmGmAmAmUmGmA*mC*mAP |
| | SEQ ID NO: 408 | PmU*fG*mUmCmAfUmUmCmUmUmGmAmUfCmUfCmAmUmCmUmA*mU*mU |
| D: 147 | SEQ ID NO: 409 | mU*mU*mGmAmCmUfUmGfAfGfUmAmAmAmUmAmUmAmU*mC*mAP |
| | SEQ ID NO: 410 | PmU*fG*mAmUmAfUmAmUmUmUmAmCmUfCmAfAmGmUmCmAmA*mC*mA |
| D: 148 | SEQ ID NO: 411 | mA*mG*mUmAmAmAfUmAfUfAfUmCmAmCmCmAmCmUmA*mU*mAP |
| | SEQ ID NO: 412 | PmU*fA*mUmAmGfUmGmGmUmGmAmUmAfUmAfUmUmUmAmCmU*mC*mA |
| D: 149 | SEQ ID NO: 413 | mU*mA*mCmCmAmAfGmUfGfCfAmGmAmUmGmUmAmAmA*mC*mAP |
| | SEQ ID NO: 414 | PmU*fG*mUmUmUfAmCmAmUmCmUmGmCfAmCfUmUmGmGmUmA*mU*mU |
| D: 150 | SEQ ID NO: 415 | mC*mU*mGmUmAmUfGmUfGfAfAmCmAmCmUmGmUmGmA*mC*mAP |
| | SEQ ID NO: 416 | PmU*FG*mUmCmAfCmAmGmUmGmUmUmCfAmCfAmUmAmCmAmG*mU*mA |
| D: 151 | SEQ ID NO: 417 | mG*mA*mUmCmAmAfGmAfAfUfGmAmCmAmAmUmGmUmC*mC*mAP |
| | SEQ ID NO: 418 | PmU*FG*mGmAmCfAmUmUmGmUmCmAmUfUmCfUmUmGmAmUmC*mU*mC |
| D: 152 | SEQ ID NO: 419 | mA*mU*mGmAmCmAfCmUfAfAfGmUmCmAmAmGmUmUmA*mA*mAP |
| | SEQ ID NO: 420 | PmU*fUmUmAmAfCmUmUmGmAmCmUmUfAmGfUmGmUmCmAmU*mG*mA |
| D: 153 | SEQ ID NO: 421 | mA*mA*mAmUmAmGfAmUfGfAfGmAmUmCmAmAmGmAmA*mU*mAP |
| | SEQ ID NO: 422 | PmU*fA*mUmUmCfUmUmGmAmUmCmUmCfAmUfCmUmAmUmUmU*mU*mG |
| D: 154 | SEQ ID NO: 423 | mG*mU*mAmUmGmUfGmAfAfCfAmCmUmGmUmGmAmCmC*mC*mAP |
| | SEQ ID NO: 424 | PmU*fG*mGmGmUfCmAmCmAmGmUmGmUfUmCfAmCmAmUmAmC*mA*mG |
| D: 155 | SEQ ID NO: 425 | mA*mA*mUmAmCmCfAmAfGfUfGmCmAmGmAmUmGmUmA*mA*mAP |
| | SEQ ID NO: 426 | PmU*U*mUmAmCfAmUmCmUmGmCmAmCfUmUfGmGmUmAmUmU*mC*mU |
| D: 156 | SEQ ID NO: 427 | mU*mG*mAmGmAmUfCmAfAfGfAmAmUmGmAmCmAmAmU*mG*mAP |
| | SEQ ID NO: 428 | PmU*fC*mAmUmUfGmUmCmAmUmUmCmUfUmGfAmUmCmUmCmA*mU*mC |
| D: 157 | SEQ ID NO: 429 | mC*mU*mUmUmGmUfAmCfUfCfUmUmGmCmAmGmAmGmA*mA*mAP |
| | SEQ ID NO: 430 | PmU*fU*mUmCmUfCmUmGmCmAmAmGmAfGmUfAmCmAmAmAmG*mA*mU |
| D: 158 | SEQ ID NO: 431 | mUmG*mAmAmGmCfCmAfAfAfAmUmAmGmAmUmGmAmG*mA*mAP |
| | SEQ ID NO: 432 | PmU*fU*mCmUmCfAmUmCmUmAmUmUmUfUmGfGmCmUmUmCmA*mU*mU |
| D: 159 | SEQ ID NO: 433 | mC*mA*mUmCmCmUfCmAfAfGfGmAmCmAmUmUmAmCmU*mA*mAP |
| | SEQ ID NO: 434 | PmU*fUmAmGmUfAmAmUmGmUmCmCmUfUmGfAmGmGmAmUmG*mA*mU |
| D: 160 | SEQ ID NO: 435 | mU*mA*mUmGmUmGfAmAfCfAfCmUmGmUmGmAmCmCmC*mU*mAP |
| | SEQ ID NO: 436 | PmU*fA*mGmGmGfUmCmAmCmAmGmUmGfUmUfCmAmCmAmUmA*mC*mA |
| D: 161 | SEQ ID NO: 437 | mG*mU*mGmUmUmAfAmUfGfCfCmCmAmAmGmUmGmAmC*mU*mAP |
| | SEQ ID NO: 438 | PmU*fA*mGmUmCfAmCmUmUmGmGmGmCfAmUfUmAmAmCmAmC*mU*mU |
| D: 162 | SEQ ID NO: 439 | mU*mU*mCmAmAmCfUmGfCfUfUmCmGmUmAmAmUmUmG*mG*mAP |
| | SEQ ID NO: 440 | PmU*fC*mCmAmAfUmUmAmCmGmAmAmGfCmAfGmUmUmGmAmA*mC*mU |
| D: 163 | SEQ ID NO: 441 | mU*mG*mCmAmGmAfAmGfAfUfGmUmAmGmAmUmUmGmU*mG*mAP |
| | SEQ ID NO: 442 | PmU*fC*mAmCmAfAmUmCmUmAmCmAmUfCmUfUmCmUmGmCmA*mU*mU |
| D: 164 | SEQ ID NO: 443 | mG*mA*mGmUmAmAfAmUfAfUfAmUmCmAmCmCmAmCmU*mA*mAP |
| | SEQ ID NO: 444 | PmU*FU*mAmGmUfGmGmUmGmAmUmAmUfAmUfUmUmAmCmUmC*mA*mA |
| D: 165 | SEQ ID NO: 445 | mC*mA*mAmGmGmAfAmUfGfCfAmCmAmCmUmCmAmCmC*mA*mAP |
| | SEQ ID NO: 446 | PmU*fU*mGmGmUfGmAmGmUmGmUmGmCfAmUfUmCmCmUmUmG*mA*mU |
| D: 166 | SEQ ID NO: 447 | mC*mA*mGmGmUmGfAmAfAfGfGmAmAmAmGmCmUmAmG*mG*mAP |
| | SEQ ID NO: 448 | PmU*fC*mCmUmAfGmCmUmUmUmCmCmUfUmUfCmAmCmCmUmG*mG*mA |
| D: 167 | SEQ ID NO: 449 | mA*mA*mUmGmAmCfAmAfUfGfUmCmCmAmAmGmAmCmA*mC*mAP |
| | SEQ ID NO: 450 | PmU*fG*mUmGmUfCmUmUmGmGmAmCmAfUmUfGmUmCmAmUmU*mC*mU |
| D: 168 | SEQ ID NO: 451 | mG*mG*mAmAmUmGfCmAfCfAfCmUmCmAmCmCmAmGmC*mA*mAP |
| | SEQ ID NO: 452 | PmU*fU*mGmCmUfGmGmUmGmAmGmUmGfUmGfCmAmUmUmCmC*mU*mU |
| D: 169 | SEQ ID NO: 453 | mC*mC*mUmGmUmCfCmUfCfCfAmGmGmUmGmAmAmAmG*mG*mAP |
| | SEQ ID NO: 454 | PmU*C*mCmUmUfUmCmAmCmCmUmGmGfAmGfGmAmCmAmGmG*mG*mC |
| D: 170 | SEQ ID NO: 455 | mA*mU*mGmCmAmGfAmAfGfAfUmGmUmAmGmAmUmUmG*mU*mAP |
| | SEQ ID NO: 456 | PmU*A*mCmAmAfUmCmUmAmCmAmUmCfUmUfCmUmGmCmAmU*mU*mU |
| D: 171 | SEQ ID NO: 457 | mU*mU*mGmUmGmUfGmAfUfGfAmAmGmGmAmCmAmUmG*mG*mAP |
| | SEQ ID NO: 458 | PmU*fC*mCmAmUfGmUmCmCmUmUmCmAfUmCfAmCmAmCmAmA*mU*mC |
| D: 172 | SEQ ID NO: 459 | mA*mA*mAmGmUmUfCmAfAfCfUmGmCmUmUmCmGmUmA*mA*mAP |
| | SEQ ID NO: 460 | PmU*fU*mUmAmCfGmAmAmGmCmAmGmUfUmGfAmAmCmUmUmU*mC*mU |
| D: 173 | SEQ ID NO: 461 | mU*mG*mUmUmAmAfUmGfCfCfCmAmAmGmUmGmAmCmU*mG*mAP |
| | SEQ ID NO: 462 | PmU*fC*mAmGmUfCmAmCmUmUmGmGmGfCmAfUmUmAmAmCmA*mC*mU |
| D: 174 | SEQ ID NO: 463 | mG*mA*mUmGmUmUfGmAfCfUfUmGmAmGmUmAmAmAmU*mA*mAP |
| | SEQ ID NO: 464 | PmU*fU*mAmUmUfUmAmCmUmCmAmAmGfUmCfAmAmCmAmUmC*mA*mG |
| D: 175 | SEQ ID NO: 465 | mG*mA*mAmGmCmCfAmAfAfAfUmAmGmAmUmGmAmGmA*mU*mAP |
| | SEQ ID NO: 466 | PmU*fA*mUmCmUfCmAmUmCmUmAmUmUfUmUfGmGmCmUmUmC*mA*mU |
| D: 176 | SEQ ID NO: 467 | mG*mU*mUmCmAmAfCmUfGfCfUmUmCmGmUmAmAmUmU*mG*mAP |
| | SEQ ID NO: 468 | PmU*fC*mAmAmUfUmAmCmGmAmAmGmCfAmGfUmUmGmAmAmC*mU*mU |
| D: 177 | SEQ ID NO: 469 | mG*mU*mAmCmUmCfUmUfGfCfAmGmAmGmAmAmAmAmU*mU*mAP |
| | SEQ ID NO: 470 | PmU*A*mAmUmUfUmUmCmUmCmUmGmCfAmAfGmAmGmUmAmC*mA*mA |
| D: 178 | SEQ ID NO: 471 | mC*mU*mCmAmAmGfGmAfCfAfUmUmAmCmUmAmGmUmG*mA*mAP |
| | SEQ ID NO: 472 | PmU*U*mCmAmCfUmAmGmUmAmAmUmGfUmCfCmUmUmGmAmG*mG*mA |
| D: 179 | SEQ ID NO: 473 | mA*mG*mGmGmAmAfGmGfAfGfUmAmCmAmCmAmGmAmC*mA*mAP |
| | SEQ ID NO: 474 | PmU*fU*mGmUmCfUmGmUmGmUmAmCmUfCmCfUmUmCmCmCmU*mU*mC |
| D: 180 | SEQ ID NO: 475 | mUmC*mAmUmGmAfCmAfCfUfAmAmGmUmCmAmAmGmU*mU*mAP |
| | SEQ ID NO: 476 | PmU*fA*mAmCmUfUmGmAmCmUmUmAmGfUmGfUmCmAmUmGmA*mC*mU |
| D: 181 | SEQ ID NO: 477 | mC*mC*mUmCmAmAfGmGfAfCfAmUmUmAmCmUmAmGmU*mG*mAP |
| | SEQ ID NO: 478 | PmU*fC*mAmCmUfAmGmUmAmAmUmGmUfCmCfUmUmGmAmGmG*mA*mU |
| D: 182 | SEQ ID NO: 479 | mG*mG*mCmUmUmUfGmUfUfCfGmAmAmAmGmAmAmUmG*mG*mAP |
| | SEQ ID NO: 480 | PmU*fC*mCmAmUfUmCmUmUmUmCmGmAfAmCfAmAmAmGmCmC*mU*mU |
| D: 183 | SEQ ID NO: 481 | mC*mC*mAmGmAmCfUmGfCfGfUmGmCmCmCmUmGmCmC*mA*mAP |
| | SEQ ID NO: 482 | PmU*fU*mGmGmCfAmGmGmGmCmAmCmGfCmAfGmUmCmUmGmG*mU*mU |
| D: 184 | SEQ ID NO: 483 | mA*mG*mCmCmAmAfAmAfUfAfGmAmUmGmAmGmAmUmC*mA*mAP |
| | SEQ ID NO: 484 | PmU*fUmGmAmUfCmUmCmAmUmCmUmAfUmUfUmUmGmGmCmU*mU*mC |
| D: 185 | SEQ ID NO: 485 | mA*mG*mAmAmGmGfGmAfAfGfGmAmGmUmAmCmAmCmA*mG*mAP |
| | SEQ ID NO: 486 | PmU*fC*mUmGmUfGmUmAmCmUmCmCmUfUmCfCmCmUmUmCmU*mU*mG |
| D: 186 | SEQ ID NO: 487 | mA*mG*mAmUmUmGfUmGfUfGfAmUmGmAmAmGmGmAmC*mA*mAP |
| | SEQ ID NO: 488 | PmU*fU*mGmUmCfCmUmUmCmAmUmCmAfCmAfCmAmAmUmCmU*mA*mC |
| D: 187 | SEQ ID NO: 489 | mU*mU*mUmCmAmCfUmUfCfGfGmAmGmGmAmUmUmGmC*mU*mAP |
| | SEQ ID NO: 490 | PmU*fA*mGmCmAfAmUmCmCmUmCmCmGfAmAfGmUmGmAmAmA*mG*mA |
| D: 188 | SEQ ID NO: 491 | mA*mA*mGmGmAmAfAmGfCfUfAmGmGmGmAmCmUmGmC*mA*mAP |
| | SEQ ID NO: 492 | PmU*fU*mGmCmAfGmUmCmCmCmUmAmGfCmUfUmUmCmCmUmU*mU*mC |
| D: 189 | SEQ ID NO: 493 | mG*mA*mCmAmUmGfGmCfUfUfAmGmAmAmGmUmGmGmA*mA*mAP |
| | SEQ ID NO: 494 | PmU*fU*mUmCmCfAmCmUmUmCmUmAmAfGmCfCmAmUmGmUmC*mC*mU |
| D: 190 | SEQ ID NO: 495 | mU*mG*mUmAmCmUfCmUfUfGfCmAmGmAmGmAmAmAmA*mU*mAP |
| | SEQ ID NO: 496 | PmU*A*mUmUmUfUmCmUmCmUmGmCmAfAmGfAmGmUmAmCmA*mA*mA |
| D: 191 | SEQ ID NO: 497 | mA*mU*mGmAmAmCfCmAfGfAfCmUmGmCmGmUmGmCmC*mC*mAP |
| | SEQ ID NO: 498 | PmU*fG*mGmGmCfAmCmGmCmAmGmUmCfUmGfGmUmUmCmAmU*mC*mC |
| D: 192 | SEQ ID NO: 499 | mC*mA*mUmGmAmCfAmCfUfAfAmGmUmCmAmAmGmUmU*mA*mAP |
| | SEQ ID NO: 500 | PmU*fUmAmAmCfUmUmGmAmCmUmUmAfGmUfGmUmCmAmUmG*mA*mC |
| D: 193 | SEQ ID NO: 501 | mU*mU*mGmUmUmCfGmAfAfAfGmAmAmUmGmGmUmGmU*mC*mAP |
| | SEQ ID NO: 502 | PmU*fG*mAmCmAfCmCmAmUmUmCmUmUfUmCfGmAmAmCmAmA*mA*mG |
| D: 194 | SEQ ID NO: 503 | mA*mC*mAmCmUmGfUmGfAfCfCmCmUmUmGmCmAmCmC*mA*mAP |
| | SEQ ID NO: 504 | PmU*FU*mGmGmUfGmCmAmAmGmGmGmUfCmAfCmAmGmUmGmU*mU*mC |
| D: 195 | SEQ ID NO: 505 | mG*mG*mAmAmAmUfAmAfAfCfUmGmCmAmCmCmCmGmG*mA*mAP |
| | SEQ ID NO: 506 | PmU*fU*mCmCmGfGmGmUmGmCmAmGmUfUmUfAmUmUmUmCmC*mA*mC |
| D: 196 | SEQ ID NO: 507 | mA*mA*mGmUmGmUfUmAfAfUfGmCmCmCmAmAmGmUmG*mA*mAP |
| | SEQ ID NO: 508 | PmU*fU*mCmAmCfUmUmGmGmGmCmAmUfUmAfAmCmAmCmUmU*mU*mU |
| D: 197 | SEQ ID NO: 509 | mA*mG*mUmUmCmAfAmCfUfGfCmUmUmCmGmUmAmAmU*mU*mAP |
| | SEQ ID NO: 510 | PmU*fA*mAmUmUfAmCmGmAmAmGmCmAfGmUfUmGmAmAmCmU*mU*mU |
| D: 198 | SEQ ID NO: 511 | mC *mA*mCmUmUmCfGmGfAfGfGmAmUmUmGmCmUmCmA*mA*mAP |
| | SEQ ID NO: 512 | PmU*FU*mUmGmAfGmCmAmAmUmCmCmUfCmCfGmAmAmGmUmG*mA*mA |
| D: 199 | SEQ ID NO: 513 | mA*mU*mGmAmAmGfCmCfAfAfAmAmUmAmGmAmUmGmA*mG*mAP |
| | SEQ ID NO: 514 | PmU*fC*mUmCmAfUmCmUmAmUmUmUmUfGmGfCmUmUmCmAmU*mU*mG |
| D: 200 | SEQ ID NO: 515 | mA*mC*mUmUmCmGfGmAfGfGfAmUmUmGmCmUmCmAmA*mC*mAP |
| | SEQ ID NO: 516 | PmU*FG*mUmUmGfAmGmCmAmAmUmCmCfUmCfCmGmAmAmGmU*mG*mA |
| D: 201 | SEQ ID NO: 517 | mA*mG*mGmUmGmAfAmAfGfGfAmAmAmGmCmUmAmGmG*mG*mAP |
| | SEQ ID NO: 518 | PmU*fC*mCmCmUfAmGmCmUmUmUmCmCfUmUfUmCmAmCmCmU*mG*mG |
| D: 202 | SEQ ID NO: 519 | mA*mA*mGmGmGmAfAmGfGfAfGmUmAmCmAmCmAmGmA*mC*mAP |
| | SEQ ID NO: 520 | PmU*fG*mUmCmUfGmUmGmUmAmCmUmCfCmUfUmCmCmCmUmU*mC*mU |
| D: 203 | SEQ ID NO: 521 | mA*mG*mUmGmGmAfAmAfUfAfAmAmCmUmGmCmAmCmC*mC*mAP |
| | SEQ ID NO: 522 | PmU*fG*mGmGmUfGmCmAmGmUmUmUmAfUmUfUmCmCmAmCmU*mU*mC |
| D: 204 | SEQ ID NO: 523 | mA*mU*mGmAmCmAfAmUfGfUfCmCmAmAmGmAmCmAmC*mA*mAP |
| | SEQ ID NO: 524 | PmU* fU*mGmUmGfUmCmUmUmGmGmAmCfAmUfUmGmUmCmAmU*mU*mC |
| D: 205 | SEQ ID NO: 525 | mG*mA*mUmUmGmUfGmUfGfAfUmGmAmAmGmGmAmCmA*mU*mAP |
| | SEQ ID NO: 526 | PmU*fA*mUmGmUfCmCmUmUmCmAmUmCfAmCfAmCmAmAmUmC*mU*mA |
| D: 206 | SEQ ID NO: 527 | mG*mG*mGmGmAmUfGmAfAfCfCmAmGmAmCmUmGmCmG*mU*mAP |
| | SEQ ID NO: 528 | PmU*fA*mCmGmCfAmGmUmCmUmGmGmUfUmCfAmUmCmCmCmC*mA*mU |
| D: 207 | SEQ ID NO: 529 | mG*mU*mUmAmAmUfGmCfCfCfAmAmGmUmGmAmCmUmG*mA*mAP |
| | SEQ ID NO: 530 | PmU*fU*mCmAmGfUmCmAmCmUmUmGmGfGmCfAmUmUmAmAmC*mA*mC |
| D: 208 | SEQ ID NO: 531 | mA*mC*mCmAmGmAfCmUfGfCfGmUmGmCmCmCmUmGmC*mC*mAP |
| | SEQ ID NO: 532 | PmU*fG*mGmCmAfGmGmGmCmAmCmGmCfAmGfUmCmUmGmGmU*mU*mC |
| D: 209 | SEQ ID NO: 533 | mA*mA*mCmAmCmUfGmUfGfAfCmCmCmUmUmGmCmAmC*mC*mAP |
| | SEQ ID NO: 534 | PmU*fG*mGmUmGfCmAmAmGmGmGmUmCfAmCfAmGmUmGmUmU*mC*mA |
| D: 210 | SEQ ID NO: 535 | mG*mG*mAmAmGmGfAmGfUfAfCmAmCmAmGmAmCmAmA*mA*mAP |
| | SEQ ID NO: 536 | PmU*fU*mUmUmGfUmCmUmGmUmGmUmAfCmUfCmCmUmUmCmC*mC*mU |
| D: 211 | SEQ ID NO: 537 | mG*mG*mGmAmUmGfAmAfCfCfAmGmAmCmUmGmCmGmU*mG*mAP |
| | SEQ ID NO: 538 | PmU*fC*mAmCmGfCmAmGmUmCmUmGmGfUmUfCmAmUmCmCmC*mC*mA |
| D: 212 | SEQ ID NO: 539 | mC*mU*mGmCmGmUfGmCfCfCfUmGmCmCmAmAmGmAmA*mG*mAP |
| | SEQ ID NO: 540 | PmU*fC*mUmUmCfUmUmGmGmCmAmGmGfGmCfAmCmGmCmAmG*mU*mC |
| D: 213 | SEQ ID NO: 541 | mU*mG*mAmAmCmAfCmUfGfUfGmAmCmCmCmUmUmGmC*mA*mAP |
| | SEQ ID NO: 542 | PmU*fU*mGmCmAfAmGmGmGmUmCmAmCfAmGfUmGmUmUmCmA*mC*mA |
| D: 214 | SEQ ID NO: 543 | mA*mG*mGmAmUmUfGmCfUfCfAmAmCmAmAmCmCmAmU*mG*mAP |
| | SEQ ID NO: 544 | PmU*fC*mAmUmGfGmUmUmGmUmUmGmAfGmCfAmAmUmCmCmU*mC*mC |
| D: 215 | SEQ ID NO: 545 | mU*mG*mAmCmAmCfUmAfAfGfUmCmAmAmGmUmUmAmA*mA*mAP |
| | SEQ ID NO: 546 | PmU*fU*mUmUmAfAmCmUmUmGmAmCmUfUmAfGmUmGmUmCmA*mU*mG |
| D: 216 | SEQ ID NO: 547 | mU*mG*mAmAmAmGfGmAfAfAfGmCmUmAmGmGmGmAmC*mU*mAP |
| | SEQ ID NO: 548 | PmU*fA*mGmUmCfCmCmUmAmGmCmUmUfUmCfCmUmUmUmCmA*mC*mC |
| D: 217 | SEQ ID NO: 549 | mA*mC*mAmAmUmGfUmCfCfAfAmGmAmCmAmCmAmGmC*mA*mAP |
| | SEQ ID NO: 550 | PmU*fU*mGmCmUfGmUmGmUmCmUmUmGfGmAfCmAmUmUmGmU*mC*mA |
| D: 218 | SEQ ID NO: 551 | mG*mU*mGmAmAmAfGmGfAfAfAmGmCmUmAmGmGmGmA*mC*mAP |
| | SEQ ID NO: 552 | PmU*FG*mUmCmCfCmUmAmGmCmUmUmUfCmCfUmUmUmCmAmC*mC*mU |
| D: 219 | SEQ ID NO: 553 | mA*mG*mCmUmCmUfUmUfCfAfCmUmUmCmGmGmAmGmG*mA*mAP |
| | SEQ ID NO: 554 | PmU*fU*mCmCmUfCmCmGmAmAmGmUmGfAmAfAmGmAmGmCmU*mU*mC |
| D: 220 | SEQ ID NO: 555 | mU*mU*mCmUmUmCfCmAfAfAfUmGmCmAmGmAmAmGmA*U*mAP |
| | SEQ ID NO: 556 | PmU*fA*mUmCmUfUmCmUmGmCmAmUmUfUmGfGmAmAmGmAmA*mA*mA |
| D: 221 | SEQ ID NO: 557 | mG*mG*mAmGmGmAfUmUfGfCfUmCmAmAmCmAmAmCmC*mA*mAP |
| | SEQ ID NO: 558 | PmU*fU*mGmGmUfUmGmUmUmGmAmGmCfAmAfUmCmCmUmCmC*mG*mA |
| D: 222 | SEQ ID NO: 559 | mU*mG*mCmCmAmAfGmAfAfGfGmGmAmAmGmGmAmGmU*mA*mAP |
| | SEQ ID NO: 560 | PmU*fU*mAmCmUfCmCmUmUmCmCmCmUfUmCfUmUmGmGmCmA*mG*mG |
| D: 223 | SEQ ID NO: 561 | mG*mA*mAmAmGmUfUmCFA-(AP)-fCmUmGmCmUmUmCmGmU*mA*mAP |
| | SEQ ID NO: 562 | mU*fU*mAmCmGmAmAmGmCmAmGmUmUfGmAfAmCmUmUmUmC*mU*mG |
| D: 224 | SEQ ID NO: 561 | mG*mA*mAmAmGmUfUmCFA-(AP)-fCmUmGmCmUmUmCmGmU*mA*mAP |
| | SEQ ID NO: 563 | mU*U*mAmCfGmAfAmGmCmAmGmUmUfGmAfAmCmUmUmUmC*mU*mG |
| D: 225 | SEQ ID NO: 561 | mG*mA*mAmAmGmUfUmCFA-(AP)-fCmUmGmCmUmUmCmGmU*mA*mAP |
| | SEQ ID NO: 571 | mU*fU*fAmCmGmAfAmGmCmAmGmUmUfGmAfAmCmUmUmUmC*mU*mG |
| D: 226 | SEQ ID NO: 564 | mG*mA*mAmAmGmUmUmCfAfAfCmUmGmCmUmUmCmGmU*mA*mAP |
| | SEQ ID NO: 563 | mU*fU*mAmCfGmAfAmGmCmAmGmUmUfGmAfAmCmUmUmUmC*mU*mG |
| D: 227 | SEQ ID NO: 564 | mG*mA*mAmAmGmUmUmCfAfAfCmUmGmCmUmUmCmGmU*mA*mAP |
| | SEQ ID NO: 571 | mU*fU*fAmCmGmAfAmGmCmAmGmUmUfGmAfAmCmUmUmUmC*mU*mG |
| D: 228 | SEQ ID NO: 565 | mG*mU*mUmGmAmC fUmUfG-(AP)-fGmUmAmAmAmUmAmUmA*mU*mAP |
| | SEQ ID NO: 566 | mU*fA*mUmAfUmAfUmUmUmAmCmUmCfAmAfGmUmCmAmAmC*mA*mU |
| D: 229 | SEQ ID NO: 565 | mG*mU*mUmGmAmC fUmUfG-(AP)-fGmUmAmAmAmUmAmUmA*mU*mAP |
| | SEQ ID NO: 567 | mU*fA*fUmAmUmAfUmUmUmAmCmUmCfAmAfGmUmCmAmAmC*mA*mU |
| D: 230 | SEQ ID NO: 568 | mG*mU*mUmGmAmCmUmUfGfAfGmUmAmAmAmUmAmUmA*mU*mAP |
| | SEQ ID NO: 566 | mU*fA*mUmAfUmAfUmUmUmAmCmUmCfAmAfGmUmCmAmAmC*mA*mU |
| D: 231 | SEQ ID NO: 568 | mG*mUmUmGmAmCmUmUfGfAfGmUmAmAmAmUmAmUmA*mU*mAP |
| | SEQ ID NO: 567 | mU*fA*fUmAmUmAfUmUmUmAmCmUmCfAmAfGmUmCmAmAmC*mA*mU |
| D: 232 | SEQ ID NO: 345 | mG*mU*mUmGmAmCfUmUfGfAfGmUmAmAmAmUmAmUmA*mU*mAP |
| | SEQ ID NO: 569 | mU*fA*mUmAfU-GNA(A)- |
| | | fUmUmUmAmCmUmCfAmAfGmUmCmAmAmC*mA*mU |
| D: 233 | SEQ ID NO: 345 | mG*mUmUmGmAmCfUmUfGfAfGmUmAmAmAmUmAmUmA*mU*mAP |
| | SEQ ID NO: 570 | mU*fA*mUmAmUFA-GNA(U)- |
| | | mUmUmAmCmUmCfAmAfGmUmCmAmAmC*mA*mU |
| D: 237 | SEQ ID NO: 578 | mA*mG*mAmAmAmGfUmUfCfAfAmCmUmGmCmUmUmCmG*mU*mAP |
| | SEQ ID NO: 579 | mU*fA*mCmGmAfAmGmCmAmGmUmUmGfAmAfCmUmUmUmCmU*mG*mU |
| D: 238 | SEQ ID NO: 580 | mC *mUmGmUmAmUmGmUfGfAfAmCmAmCmUmGmUmGmA*mC*mAP |
| | SEQ ID NO: 581 | mU*fG*fUmCmAmCfAmGmUmGmUmUmCfAmCfAmUmAmCmAmG*mU*mA |
| D: 239 | SEQ ID NO: 582 | mU*mG*mAmCmUmUmGmAfGfUfAmAmAmUmAmUmAmUmC*mA*mAP |
| | SEQ ID NO: 583 | mU*fU*fGmAmUmAfUmAmUmUmUmAmCfUmCfAmAmGmUmCmA*mA*mC |
| D: 240 | SEQ ID NO: 584 | mG*mU*mAmUmGmUmGmAfAfCfAmCmUmGmUmGmAmCmC*mC*mAP |
| | SEQ ID NO: 585 | mU*fG*fGmGmUmCfAmCmAmGmUmGmUfUmCfAmCmAmUmAmC*mA*mG |

P indicates a 5′ phosphate;
m indicates 2′O-methyl modified ribose on the listed nucleotide;
f indicates 2′F modified ribose on the listed nucleotide;
*indicates a phosphorothioate bond (in place of a phosphodiester bond);
GNA indicates a glycol nucleic acid nucleotide;
(AP) means an apurinic/apyrimidinic residue, also called an abasic residue.

TABLE 4B
FAS-GalNAc RNAi agents, modified sense and antisense strands

| Duplex | SEQ ID NO: | Modified Sequence |
|---|---|---|
| D: | SEQ ID NO: 586 | mG*mA*mUmGmAmAfGmGfAfCfAmUmGmGmCmUmUmAmG*mA*mA |
| 241 | SEQ ID NO: 587 | mU*fU*mCmUmAfAmGmCmCmAmUmGmUfCmCfUmUmCmAmUmC*mA*mC |
| D: | SEQ ID NO: 588 | mU*mA*mAmAmCmCfAmAfAfCfUmUmUmUmUmUmUmGmU*mA*mA |
| 242 | SEQ ID NO: 589 | mU*fU*mAmCmAfAmAmAmAmAmAmGmUfUmUfGmGmUmUmUmA*mC*mA |
| D: | SEQ ID NO: 590 | mG*mA*mAmGmAmUfGmUfAfGfAmUmUmGmUmGmUmGmA*mU*mA |
| 243 | SEQ ID NO: 591 | mU*fAmUmCmAfCmAmCmAmAmUmCmUfAmCfAmUmCmUmUmC*mU*mG |
| D: | SEQ ID NO: 592 | mG*mU*mUmGmAmCfUmUfGfAfGmUmAmAmAmUmAmUmA*mU*mA |
| 244 | SEQ ID NO: 593 | mU*fA*mUmAmUfAmUmUmUmAmCmUmCfAmAfGmUmCmAmAmC*mA*mU |
| D: | SEQ ID NO: 594 | mA*mU*mCmAmAmGfAmAfUfGfAmCmAmAmUmGmUmCmC*mA*mA |
| 245 | SEQ ID NO: 595 | mU*fU*mGmGmAfCmAmUmUmGmUmCmAfUmUfCmUmUmGmAmU*mC*mU |
| D: | SEQ ID NO: 596 | mU*mG*mUmUmGmAfCmUfUfGfAmGmUmAmAmAmUmAmU*mA*mA |
| 246 | SEQ ID NO: 597 | mU*fU*mAmUmAfUmUmUmAmCmUmCmAfAmGfUmCmAmAmCmA*mU*mC |
| D: | SEQ ID NO: 598 | mG*mC*mCmAmAmAfAmUfAfGfAmUmGmAmGmAmUmCmA*mA*mA |
| 247 | SEQ ID NO: 599 | mU*fU*mUmGmAfUmCmUmCmAmUmCmUfAmUfUmUmUmGmGmC*mU*mU |
| | SEQ ID NO: 600 | mA*mA*mUmGmCmAfGmAfAfGfAmUmGmUmAmGmAmUmU*mG*mA |
| D: | SEQ ID NO: 601 | mU*fC*mAmAmUfCmUmAmCmAmUmCmUfUmCfUmGmCmAmUmU*mU*mG |
| 248 | | |
| D: | SEQ ID NO: 602 | mG*mC*mUmUmUmGfUmUfCfGfAmAmAmGmAmAmUmGmG*mU*mA |
| 249 | SEQ ID NO: 603 | mU*fA*mCmCmAfUmUmCmUmUmUmCmGfAmAfCmAmAmAmGmC*mC*mU |
| D: | SEQ ID NO: 604 | mG*mA*mCmUmUmGfAmGfUfAfAmAmUmAmUmAmUmCmA*mC*mA |
| 250 | SEQ ID NO: 605 | mU*fG*mUmGmAfUmAmUmAmUmUmUmAfCmUfCmAmAmGmUmC*mA*mA |
| D: | SEQ ID NO: 606 | mA*mG*mAmUmGmAfGmAfUfCfAmAmGmAmAmUmGmAmC*mA*mA |
| 251 | SEQ ID NO: 607 | mU*fU*mGmUmCfAmUmUmCmUmUmGmAfUmCfUmCmAmUmCmU*mA*mU |
| D: | SEQ ID NO: 608 | mU*mU*mGmAmGmUfAmAfAfUfAmUmAmUmCmAmCmCmA*mC*mA |
| 252 | SEQ ID NO: 609 | mU*fG*mUmGmGfUmGmAmUmAmUmAmUfUmUfAmCmUmCmAmA*mG*mU |
| D: | SEQ ID NO: 610 | mA*mA*mUmAmGmAfUmGfAfGfAmUmCmAmAmGmAmAmU*mG*mA |
| 253 | SEQ ID NO: 611 | mU*fC*mAmUmUfCmUmUmGmAmUmCmUfCmAfUmCmUmAmUmU*mU*mU |
| D: | SEQ ID NO: 612 | mC *mUmUmUmGmUfUmCfGfAfAmAmGmAmAmUmGmGmU*mG*mA |
| 254 | SEQ ID NO: 613 | mU*fC*mAmCmCfAmUmUmCmUmUmUmCfGmAfAmCmAmAmAmG*mC*mC |
| D: | SEQ ID NO: 614 | mU*mG*mUmGmAmUfGmAfAfGfGmAmCmAmUmGmGmCmU*mU*mA |
| 255 | SEQ ID NO: 615 | mU*fA*mAmGmCfCmAmUmGmUmCmCmUfUmCfAmUmCmAmCmA*mC*mA |
| D: | SEQ ID NO: 616 | mUmG*mAmCmUmUfGmAfGfUfAmAmAmUmAmUmAmUmC*mA*mA |
| 256 | SEQ ID NO: 617 | mU*fU*mGmAmUfAmUmAmUmUmUmAmCfUmCfAmAmGmUmCmA*mA*mC |
| D: | SEQ ID NO: 618 | mG*mA*mAmAmGmUfUmCfAfAfCmUmGmCmUmUmCmGmU*mA*mA |
| 257 | SEQ ID NO: 619 | mU*fU*mAmCmGfAmAmGmCmAmGmUmUfGmAfAmCmUmUmUmC*mU*mG |
| D: | SEQ ID NO: 620 | mG*mC*mAmGmAmAfGmAfUfGfUmAmGmAmUmUmGmUmG*mU*mA |
| 258 | SEQ ID NO: 621 | mU*fAmCmAmCfAmAmUmCmUmAmCmAfUmCfUmUmCmUmGmC*mA*mU |
| D: | SEQ ID NO: 622 | mU*mG*mUmGmAmAfCmAfCfUfGmUmGmAmCmCmCmUmUmG*mA |
| 259 | SEQ ID NO: 623 | mU*C*mAmAmGfGmGmUmCmAmCmAmGfUmGfUmUmCmAmCmA*mU*mA |
| D: | SEQ ID NO: 624 | mG*mA*mCmAmCmUfAmAfGfUfCmAmAmGmUmUmAmAmA*mG*mA |
| 260 | SEQ ID NO: 625 | mU*fC*mUmUmUfAmAmCmUmUmGmAmCfUmUfAmGmUmGmUmC*mA*mU |
| D: | SEQ ID NO: 626 | mG*mA*mAmUmAmCfCmAfAfGfUmGmCmAmGmAmUmGmU*mA*mA |
| 261 | SEQ ID NO: 627 | mU*fU*mAmCmAfUmCmUmGmCmAmCmUfUmGfGmUmAmUmUmC*mU*mG |
| D: | SEQ ID NO: 628 | mG*mA*mCmAmAmUfGmUfCfCfAmAmGmAmCmAmCmAmG*mC*mA |
| 262 | SEQ ID NO: 629 | mU*fG*mCmUmGfUmGmUmCmUmUmGmGfAmCfAmUmUmGmUmC*mA*mU |
| D: | SEQ ID NO: 630 | mA*mU*mGmAmAmGfGmAfCfAfUmGmGmCmUmUmAmGmA*mA*mA |
| 263 | SEQ ID NO: 631 | mU*fU*mUmCmUfAmAmGmCmCmAmUmGfUmCfCmUmUmCmAmU*mC*mA |
| D: | SEQ ID NO: 632 | mU*mU*mCmCmAmAfAmUfGfCfAmGmAmAmGmAmUmGmU*mA*mA |
| 264 | SEQ ID NO: 633 | mU*fU*mAmCmAfUmCmUmUmCmUmGmCfAmUfUmUmGmGmAmA*mG*mA |
| D: | SEQ ID NO: 634 | mG*mA*mGmAmUmCfAmAfGfAfAmUmGmAmCmAmAmUmG*mU*mA |
| 265 | SEQ ID NO: 635 | mU*fA*mCmAmUfUmGmUmCmAmUmUmCfUmUfGmAmUmCmUmC*mA*mU |
| D: | SEQ ID NO: 636 | mC*mA*mAmGmUmGfCmAfGfAfUmGmUmAmAmAmCmCmA*mA*mA |
| 266 | SEQ ID NO: 637 | mU*fU*mUmGmGfUmUmUmAmCmAmUmCfUmGfCmAmCmUmUmG*mG*mU |
| D: | SEQ ID NO: 638 | mU*mU*mUmGmUmUfCmGfAfAfAmGmAmAmUmGmGmUmG*mU*mA |
| 267 | SEQ ID NO: 639 | mU*A*mCmAmCfCmAmUmUmCmUmUmUfCmGfAmAmCmAmAmA*mG*mC |
| D: | SEQ ID NO: 640 | mC*mA*mGmAmAmGfAmUfGfUfAmGmAmUmUmGmUmGmU*mG*mA |
| 268 | SEQ ID NO: 641 | mU*fC*mAmCmAfCmAmAmUmCmUmAmCfAmUfCmUmUmCmUmG*mC*mA |
| D: | SEQ ID NO: 642 | mU*mC*mUmUmUmGfUmAfCfUfCmUmUmGmCmAmGmAmG*mA*mA |
| 269 | SEQ ID NO: 643 | mU*fU*mCmUmCfUmGmCmAmAmGmAmGfUmAfCmAmAmAmGmA*mU*mU |
| D: | SEQ ID NO: 644 | mC *mA*mCmUmGmUfGmAfCfCfCmUmUmGmCmAmCmCmA*mA*mA |
| 270 | SEQ ID NO: 645 | mU*fUmUmGmGfUmGmCmAmAmGmGmGfUmCfAmCmAmGmUmG*mU*mU |
| D: | SEQ ID NO: 646 | mU*mC*mCmAmAmAfUmGfCFAfGmAmAmGmAmUmGmUmA*mG*mA |
| 271 | SEQ ID NO: 647 | mU*fC*mUmAmCfAmUmCmUmUmCmUmGfCmAfUmUmUmGmGmA*mA*mG |
| D: | SEQ ID NO: 648 | mA*mA*mUmGmUmCfCmAfAfGfAmCmAmCmAmGmCmAmG*mA*mA |
| 272 | SEQ ID NO: 649 | mU*fU*mCmUmGfCmUmGmUmGmUmCmUfUmGfGmAmCmAmUmU*mG*mU |
| D: | SEQ ID NO: 650 | mG*mC*mCmAmAmGfAmAfGfGfGmAmAmGmGmAmGmUmA*mC*mA |
| 273 | SEQ ID NO: 651 | mU*fG*mUmAmCfUmCmCmUmUmCmCmCfUmUfCmUmUmGmGmC*mA*mG |
| D: | SEQ ID NO: 652 | mC*mU*mUmGmAmGfUmAfAfAfUmAmUmAmUmCmAmCmC*mA*mA |
| 274 | SEQ ID NO: 653 | mU*fU*mGmGmUfGmAmUmAmUmAmUmUfUmAfCmUmCmAmAmG*mU*mC |
| D: | SEQ ID NO: 654 | mU*mA*mGmAmUmGfAmGfAfUfCmAmAmGmAmAmUmGmA*mC*mA |
| 275 | SEQ ID NO: 655 | mU*G*mUmCmAfUmUmCmUmUmGmAmUfCmUfCmAmUmCmUmA*mU*mU |
| D: | SEQ ID NO: 656 | mU*mU*mGmAmCmUfUmGfAfGfUmAmAmAmUmAmUmAmU*mC*mA |
| 276 | SEQ ID NO: 657 | mU*fG*mAmUmAfUmAmUmUmUmAmCmUfCmAfAmGmUmCmAmA*mC*mA |
| D: | SEQ ID NO: 658 | mA*mG*mUmAmAmAfUmAfUfAfUmCmAmCmCmAmCmUmA*mU*mA |
| 277 | SEQ ID NO: 659 | mU*fA*mUmAmGfUmGmGmUmGmAmUmAfUmAfUmUmUmAmCmU*mC*mA |
| D: | SEQ ID NO: 660 | mU*mA*mCmCmAmAfGmUfGfCfAmGmAmUmGmUmAmAmA*mC*mA |
| 278 | SEQ ID NO: 661 | mU*FG*mUmUmUfAmCmAmUmCmUmGmCfAmCfUmUmGmGmUmA*mU*mU |
| D: | SEQ ID NO: 662 | mC *mU*mGmUmAmUfGmUfGfAfAmCmAmCmUmGmUmGmA*mC*mA |
| 279 | SEQ ID NO: 663 | mU*fG*mUmCmAfCmAmGmUmGmUmUmCfAmCfAmUmAmCmAmG*mU*mA |
| D: | SEQ ID NO: 664 | mG*mA*mUmCmAmAfGmAfAfUfGmAmCmAmAmUmGmUmC*mC*mA |
| 280 | SEQ ID NO: 665 | mU*FG*mGmAmCfAmUmUmGmUmCmAmUfUmCfUmUmGmAmUmC*mU*mC |
| D: | SEQ ID NO: 666 | mA*mU*mGmAmCmAfCmUfAfAfGmUmCmAmAmGmUmUmA*mA*mA |
| 281 | SEQ ID NO: 667 | mU*U*mUmAmAfCmUmUmGmAmCmUmUfAmGfUmGmUmCmAmU*mG*mA |
| D: | SEQ ID NO: 668 | mA*mA*mAmUmAmGfAmUfGfAfGmAmUmCmAmAmGmAmA*mU*mA |
| 282 | SEQ ID NO: 669 | mU*A*mUmUmCfUmUmGmAmUmCmUmCfAmUfCmUmAmUmUmU*mU*mG |
| D: | SEQ ID NO: 670 | mG*mU*mAmUmGmUfGmAfAfCfAmCmUmGmUmGmAmCmC*mC*mA |
| 283 | SEQ ID NO: 671 | mU*fG*mGmGmUfCmAmCmAmGmUmGmUfUmCfAmCmAmUmAmC*mA*mG |
| D: | SEQ ID NO: 672 | mA*mA*mUmAmCmCfAmAfGfUfGmCmAmGmAmUmGmUmA*mA*mA |
| 284 | SEQ ID NO: 673 | mU*fU*mUmAmCfAmUmCmUmGmCmAmCfUmUfGmGmUmAmUmU*mC*mU |
| D: | SEQ ID NO: 674 | mU*mG*mAmGmAmUfCmAfAfGfAmAmUmGmAmCmAmAmU*mG*mA |
| 285 | SEQ ID NO: 675 | mU*fC*mAmUmUfGmUmCmAmUmUmCmUfUmGfAmUmCmUmCmA*mU*mC |
| D: | SEQ ID NO: 676 | mC *mU*mUmUmGmUfAmCfUfCfUmUmGmCmAmGmAmGmA*mA*mA |
| 286 | SEQ ID NO: 677 | mU*fU*mUmCmUfCmUmGmCmAmAmGmAfGmUfAmCmAmAmAmG*mA*mU |
| D: | SEQ ID NO: 678 | mU*mG*mAmAmGmCfCmAfAfAfAmUmAmGmAmUmGmAmG*mA*mA |
| 287 | SEQ ID NO: 679 | mU*fU*mCmUmCfAmUmCmUmAmUmUmUfUmGfGmCmUmUmCmA*mU*mU |
| D: | SEQ ID NO: 680 | mC*mA*mUmCmCmUfCmAfAfGfGmAmCmAmUmUmAmCmU*mA*mA |
| 288 | SEQ ID NO: 681 | mU*fU*mAmGmUfAmAmUmGmUmCmCmUfUmGfAmGmGmAmUmG*mA*mU |
| D: | SEQ ID NO: 682 | mU*mA*mUmGmUmGfAmAfCfAfCmUmGmUmGmAmCmCmC*mU*mA |
| 289 | SEQ ID NO: 683 | mU*fA*mGmGmGfUmCmAmCmAmGmUmGfUmUfCmAmCmAmUmA*mC*mA |
| D: | SEQ ID NO: 684 | mG*mUmGmUmUmAfAmUfGfCfCmCmAmAmGmUmGmAmC*mU*mA |
| 290 | SEQ ID NO: 685 | mU*fA*mGmUmCfAmCmUmUmGmGmGmCfAmUfUmAmAmCmAmC*mU*mU |
| D: | SEQ ID NO: 686 | mU*mU*mCmAmAmCfUmGfCfUfUmCmGmUmAmAmUmUmG*mG*mA |
| 291 | SEQ ID NO: 687 | mU*fC*mCmAmAfUmUmAmCmGmAmAmGfCmAfGmUmUmGmAmA*mC*mU |
| D: | SEQ ID NO: 688 | mU*mG*mCmAmGmAfAmGfAfUfGmUmAmGmAmUmUmGmU*mG*mA |
| 292 | SEQ ID NO: 689 | mU*fC*mAmCmAfAmUmCmUmAmCmAmUfCmUfUmCmUmGmCmA*mU*mU |
| D: | SEQ ID NO: 690 | mG*mA*mGmUmAmAfAmUfAfUfAmUmCmAmCmCmAmCmU*mA*mA |
| 293 | SEQ ID NO: 691 | mU*fU*mAmGmUfGmGmUmGmAmUmAmUfAmUfUmUmAmCmUmC*mA*mA |
| D: | SEQ ID NO: 692 | mC*mA*mAmGmGmAfAmUfGfCfAmCmAmCmUmCmAmCmC*mA*mA |
| 294 | SEQ ID NO: 693 | mU*FU*mGmGmUfGmAmGmUmGmUmGmCfAmUfUmCmCmUmUmG*mA*mU |
| D: | SEQ ID NO: 694 | mC*mA*mGmGmUmGfAmAfAfGfGmAmAmAmGmCmUmAmG*mG*mA |
| 295 | SEQ ID NO: 695 | mU*fC*mCmUmAfGmCmUmUmUmCmCmUfUmUfCmAmCmCmUmG*mG*mA |
| D: | SEQ ID NO: 696 | mA*mA*mUmGmAmCfAmAfUfGfUmCmCmAmAmGmAmCmA*mC*mA |
| 296 | SEQ ID NO: 697 | mU*fG*mUmGmUfCmUmUmGmGmAmCmAfUmUfGmUmCmAmUmU*mC*mU |
| D: | SEQ ID NO: 698 | mG*mG*mAmAmUmGfCmAfCfAfCmUmCmAmCmCmAmGmC*mA*mA |
| 297 | SEQ ID NO: 699 | mU*fU*mGmCmUfGmGmUmGmAmGmUmGfUmGfCmAmUmUmCmC*mU*mU |
| D: | SEQ ID NO: 700 | mC*mC *mUmGmUmCfCmUfCfCfAmGmGmUmGmAmAmAmG*mG*mA |
| 298 | SEQ ID NO: 701 | mU*fC*mCmUmUfUmCmAmCmCmUmGmGfAmGfGmAmCmAmGmG*mG*mC |
| D: | SEQ ID NO: 702 | mA*mU*mGmCmAmGfAmAfGfAfUmGmUmAmGmAmUmUmG*mU*mA |
| 299 | SEQ ID NO: 703 | mU*A*mCmAmAfUmCmUmAmCmAmUmCfUmUfCmUmGmCmAmU*mU*mU |
| D: | SEQ ID NO: 704 | mU*mU*mGmUmGmUfGmAfUfGfAmAmGmGmAmCmAmUmG*mG*mA |
| 300 | SEQ ID NO: 705 | mU*fC*mCmAmUfGmUmCmCmUmUmCmAfUmCfAmCmAmCmAmA*mU*mC |
| D: | SEQ ID NO: 706 | mA*mA*mAmGmUmUfCmAfAfCfUmGmCmUmUmCmGmUmA*mA*mA |
| 301 | SEQ ID NO: 707 | mU*fU*mUmAmCfGmAmAmGmCmAmGmUfUmGfAmAmCmUmUmU*mC*mU |
| D: | SEQ ID NO: 708 | mU*mG*mUmUmAmAfUmGfCfCfCmAmAmGmUmGmAmCmU*mG*mA |
| 302 | SEQ ID NO: 709 | mU*fC*mAmGmUfCmAmCmUmUmGmGmGfCmAfUmUmAmAmCmA*mC*mU |
| D: | SEQ ID NO: 710 | mG*mA*mUmGmUmUfGmAfCfUfUmGmAmGmUmAmAmAmU*mA*mA |
| 303 | SEQ ID NO: 711 | mU*fU*mAmUmUfUmAmCmUmCmAmAmGfUmCfAmAmCmAmUmC*mA*mG |
| D: | SEQ ID NO: 712 | mG*mA*mAmGmCmCfAmAfAfAfUmAmGmAmUmGmAmGmA*mU*mA |
| 304 | SEQ ID NO: 713 | mU*fA*mUmCmUfCmAmUmCmUmAmUmUfUmUfGmGmCmUmUmC*mA*mU |
| D: | SEQ ID NO: 714 | mG*mU*mUmCmAmAfCmUfGfCfUmUmCmGmUmAmAmUmU*mG*mA |
| 305 | SEQ ID NO: 715 | mU*C*mAmAmUfUmAmCmGmAmAmGmCfAmGfUmUmGmAmAmC*mU*mU |
| D: | SEQ ID NO: 716 | mG*mU*mAmCmUmCfUmUfGfCfAmGmAmGmAmAmAmAmU*mU*mA |
| 306 | SEQ ID NO: 717 | mU*fA*mAmUmUfUmUmCmUmCmUmGmCfAmAfGmAmGmUmAmC*mA*mA |
| D: | SEQ ID NO: 718 | mC *mU*mCmAmAmGfGmAfCfAfUmUmAmCmUmAmGmUmG*mA*mA |
| 307 | SEQ ID NO: 719 | mU*fU*mCmAmCfUmAmGmUmAmAmUmGfUmCfCmUmUmGmAmG*mG*mA |
| D: | SEQ ID NO: 720 | mA*mG*mGmGmAmAfGmGfAfGfUmAmCmAmCmAmGmAmC*mA*mA |
| 308 | SEQ ID NO: 721 | mU*fU*mGmUmCfUmGmUmGmUmAmCmUfCmCfUmUmCmCmCmU*mU*mC |
| D: | SEQ ID NO: 722 | mUmC*mAmUmGmAfCmAfCfUfAmAmGmUmCmAmAmGmU*mU*mA |
| 309 | SEQ ID NO: 723 | mU*fA*mAmCmUfUmGmAmCmUmUmAmGfUmGfUmCmAmUmGmA*mC*mU |
| D: | SEQ ID NO: 724 | mC*mC*mUmCmAmAfGmGfAfCfAmUmUmAmCmUmAmGmU*mG*mA |
| 310 | SEQ ID NO: 725 | mU*fC*mAmCmUfAmGmUmAmAmUmGmUfCmCfUmUmGmAmGmG*mA*mU |
| D: | SEQ ID NO: 726 | mG*mG*mCmUmUmUfGmUfUfCfGmAmAmAmGmAmAmUmG*mG*mA |
| 311 | SEQ ID NO: 727 | mU*fC*mCmAmUfUmCmUmUmUmCmGmAfAmCfAmAmAmGmCmC*mU*mU |
| D: | SEQ ID NO: 728 | mC*mC*mAmGmAmCfUmGfCfGfUmGmCmCmCmUmGmCmC*mA*mA |
| 312 | SEQ ID NO: 729 | mU*fU*mGmGmCfAmGmGmGmCmAmCmGfCmAfGmUmCmUmGmG*mU*mU |
| D: | SEQ ID NO: 730 | mA*mG*mCmCmAmAfAmAfUfAfGmAmUmGmAmGmAmUmC*mA*mA |
| 313 | SEQ ID NO: 731 | mU*fU*mGmAmUfCmUmCmAmUmCmUmAfUmUfUmUmGmGmCmU*mU*mC |
| D: | SEQ ID NO: 732 | mA*mG*mAmAmGmGfGmAfAfGfGmAmGmUmAmCmAmCmA*mG*mA |
| 314 | SEQ ID NO: 733 | mU*fC*mUmGmUfGmUmAmCmUmCmCmUfUmCfCmCmUmUmCmU*mU*mG |
| D: | SEQ ID NO: 734 | mA*mG*mAmUmUmGfUmGfUfGfAmUmGmAmAmGmGmAmC*mA*mA |
| 315 | SEQ ID NO: 735 | mU*fU*mGmUmCfCmUmUmCmAmUmCmAfCmAfCmAmAmUmCmU*mA*mC |
| D: | SEQ ID NO: 736 | mU*mU*mUmCmAmCfUmUfCfGfGmAmGmGmAmUmUmGmC*mU*mA |
| 316 | SEQ ID NO: 737 | mU*fA*mGmCmAfAmUmCmCmUmCmCmGfAmAfGmUmGmAmAmA*mG*mA |
| D: | SEQ ID NO: 738 | mA*mA*mGmGmAmAfAmGfCfUfAmGmGmGmAmCmUmGmC*mA*mA |
| 317 | SEQ ID NO: 739 | mU*fU*mGmCmAfGmUmCmCmCmUmAmGfCmUfUmUmCmCmUmU*mU*mC |
| D: | SEQ ID NO: 740 | mG*mA*mCmAmUmGfGmCfUfUfAmGmAmAmGmUmGmGmA*mA*mA |
| 318 | SEQ ID NO: 741 | mU*fU*mUmCmCfAmCmUmUmCmUmAmAfGmCfCmAmUmGmUmC*mC*mU |
| D: | SEQ ID NO: 742 | mU*mG*mUmAmCmUfCmUfUfGfCmAmGmAmGmAmAmAmA*mU*mA |
| 319 | SEQ ID NO: 743 | mU*fA*mUmUmUfUmCmUmCmUmGmCmAfAmGfAmGmUmAmCmA*mA*mA |
| D: | SEQ ID NO: 744 | mA*mUmGmAmAmCfCmAfGfAfCmUmGmCmGmUmGmCmC*mC*mA |
| 320 | SEQ ID NO: 745 | mU*fG*mGmGmCfAmCmGmCmAmGmUmCfUmGfGmUmUmCmAmU*mC*mC |
| D: | SEQ ID NO: 746 | mC*mA*mUmGmAmCfAmCfUfAfAmGmUmCmAmAmGmUmU*mA*mA |
| 321 | SEQ ID NO: 747 | mU*fU*mAmAmCfUmUmGmAmCmUmUmAfGmUfGmUmCmAmUmG*mA*mC |
| D: | SEQ ID NO: 748 | mU*mU*mGmUmUmCfGmAfAfAfGmAmAmUmGmGmUmGmU*mC*mA |
| 322 | SEQ ID NO: 749 | mU*fG*mAmCmAfCmCmAmUmUmCmUmUfUmCfGmAmAmCmAmA*mA*mG |
| D: | SEQ ID NO: 750 | mA*mC*mAmCmUmGfUmGfAfCfCmCmUmUmGmCmAmCmC*mA*mA |
| 323 | SEQ ID NO: 751 | mU*fU*mGmGmUfGmCmAmAmGmGmGmUfCmAfCmAmGmUmGmU*mU*mC |
| D: | SEQ ID NO: 752 | mG*mG*mAmAmAmUfAmAfAfCfUmGmCmAmCmCmCmGmG*mA*mA |
| 324 | SEQ ID NO: 753 | mU*fU*mCmCmGfGmGmUmGmCmAmGmUfUmUfAmUmUmUmCmC*mA*mC |
| D: | SEQ ID NO: 754 | mA*mA*mGmUmGmUfUmAfAfUfGmCmCmCmAmAmGmUmG*mA*mA |
| 325 | SEQ ID NO: 755 | mU*fU*mCmAmCfUmUmGmGmGmCmAmUfUmAfAmCmAmCmUmU*mU*mU |
| D: | SEQ ID NO: 756 | mA*mG*mUmUmCmAfAmCfUfGfCmUmUmCmGmUmAmAmU*mU*mA |
| 326 | SEQ ID NO: 757 | mU*fA*mAmUmUfAmCmGmAmAmGmCmAfGmUfUmGmAmAmCmU*mU*mU |
| D: | SEQ ID NO: 758 | mC*mA*mCmUmUmCfGmGfAfGfGmAmUmUmGmCmUmCmA*mA*mA |
| 327 | SEQ ID NO: 759 | mU*FU*mUmGmAfGmCmAmAmUmCmCmUfCmCfGmAmAmGmUmG*mA*mA |
| D: | SEQ ID NO: 760 | mA*mUmGmAmAmGfCmCfAfAfAmAmUmAmGmAmUmGmA*mG*mA |
| 328 | SEQ ID NO: 761 | mU*fC*mUmCmAfUmCmUmAmUmUmUmUfGmGfCmUmUmCmAmU*mU*mG |
| D: | SEQ ID NO: 762 | mA*mC*mUmUmCmGfGmAfGfGfAmUmUmGmCmUmCmAmA*mC*mA |
| 329 | SEQ ID NO: 763 | mU*fG*mUmUmGfAmGmCmAmAmUmCmCfUmCfCmGmAmAmGmU*mG*mA |
| D: | SEQ ID NO: 764 | mA*mG*mGmUmGmAfAmAfGfGfAmAmAmGmCmUmAmGmG*mG*mA |
| 330 | SEQ ID NO: 765 | mU*fC*mCmCmUfAmGmCmUmUmUmCmCfUmUfUmCmAmCmCmU*mG*mG |
| D: | SEQ ID NO: 766 | mA*mA*mGmGmGmAfAmGfGfAfGmUmAmCmAmCmAmGmA*mC*mA |
| 331 | SEQ ID NO: 767 | mU*fG*mUmCmUfGmUmGmUmAmCmUmCfCmUfUmCmCmCmUmU*mC*mU |
| D: | SEQ ID NO: 768 | mA*mG*mUmGmGmAfAmAfUfAfAmAmCmUmGmCmAmCmC*mC*mA |
| 332 | SEQ ID NO: 769 | mU*fG*mGmGmUfGmCmAmGmUmUmUmAfUmUfUmCmCmAmCmU*mU*mC |
| D: | SEQ ID NO: 770 | mA*mU*mGmAmCmAfAmUfGfUfCmCmAmAmGmAmCmAmC*mA*mA |
| 333 | SEQ ID NO: 771 | mU*fU*mGmUmGfUmCmUmUmGmGmAmCfAmUfUmGmUmCmAmU*mU*mC |
| D: | SEQ ID NO: 772 | mG*mA*mUmUmGmUfGmUfGfAfUmGmAmAmGmGmAmCmA*mU*mA |
| 334 | SEQ ID NO: 773 | mU*fA*mUmGmUfCmCmUmUmCmAmUmCfAmCfAmCmAmAmUmC*mU*mA |
| D: | SEQ ID NO: 774 | mG*mG*mGmGmAmUfGmAfAfCfCmAmGmAmCmUmGmCmG*mU*mA |
| 335 | SEQ ID NO: 775 | mU*fA*mCmGmCfAmGmUmCmUmGmGmUfUmCfAmUmCmCmCmC*mA*mU |
| D: | SEQ ID NO: 776 | mG*mU*mUmAmAmUfGmCfCfCfAmAmGmUmGmAmCmUmG*mA*mA |
| 336 | SEQ ID NO: 777 | mU*U*mCmAmGfUmCmAmCmUmUmGmGfGmCfAmUmUmAmAmC*mA*mC |
| D: | SEQ ID NO: 778 | mA*mC*mCmAmGmAfCmUfGfCfGmUmGmCmCmCmUmGmC*mC*mA |
| 337 | SEQ ID NO: 779 | mU*FG*mGmCmAfGmGmGmCmAmCmGmCfAmGfUmCmUmGmGmU*mU*mC |
| D: | SEQ ID NO: 780 | mA*mA*mCmAmCmUfGmUfGfAfCmCmCmUmUmGmCmAmC*mC*mA |
| 338 | SEQ ID NO: 781 | mU*fG*mGmUmGfCmAmAmGmGmGmUmCfAmCfAmGmUmGmUmU*mC*mA |
| D: | SEQ ID NO: 782 | mG*mG*mAmAmGmGfAmGfUfAfCmAmCmAmGmAmCmAmA*mA*mA |
| 339 | SEQ ID NO: 783 | mU*fU*mUmUmGfUmCmUmGmUmGmUmAfCmUfCmCmUmUmCmC*mC*mU |
| D: | SEQ ID NO: 784 | mG*mG*mGmAmUmGfAmAfCfCfAmGmAmCmUmGmCmGmU*mG*mA |
| 340 | SEQ ID NO: 785 | mU*fC*mAmCmGfCmAmGmUmCmUmGmGfUmUfCmAmUmCmCmC*mC*mA |
| D: | SEQ ID NO: 786 | mC *mU*mGmCmGmUfGmCfCfCfUmGmCmCmAmAmGmAmA*mG*mA |
| 341 | SEQ ID NO: 787 | mU*C*mUmUmCfUmUmGmGmCmAmGmGfGmCfAmCmGmCmAmG*mU*mC |
| D: | SEQ ID NO: 788 | mU*mG*mAmAmCmAfCmUfGfUfGmAmCmCmCmUmUmGmC*mA*mA |
| 342 | SEQ ID NO: 789 | mU*fU*mGmCmAfAmGmGmGmUmCmAmCfAmGfUmGmUmUmCmA*mC*mA |
| D: | SEQ ID NO: 790 | mA*mG*mGmAmUmUfGmCfUfCfAmAmCmAmAmCmCmAmU*mG*mA |
| 343 | SEQ ID NO: 791 | mU*C*mAmUmGfGmUmUmGmUmUmGmAfGmCfAmAmUmCmCmU*mC*mC |
| D: | SEQ ID NO: 792 | mUmG*mAmCmAmCfUmAfAfGfUmCmAmAmGmUmUmAmA*mA*mA |
| 344 | SEQ ID NO: 793 | mU*fU*mUmUmAfAmCmUmUmGmAmCmUfUmAfGmUmGmUmCmA*mU*mG |
| D: | SEQ ID NO: 794 | mU*mG*mAmAmAmGfGmAfAfAfGmCmUmAmGmGmGmAmC*mU*mA |
| 345 | SEQ ID NO: 795 | mU*fA*mGmUmCfCmCmUmAmGmCmUmUfUmCfCmUmUmUmCmA*mC*mC |
| D: | SEQ ID NO: 796 | mA*mC*mAmAmUmGfUmCfCfAfAmGmAmCmAmCmAmGmC*mA*mA |
| 346 | SEQ ID NO: 797 | mU*fU*mGmCmUfGmUmGmUmCmUmUmGfGmAfCmAmUmUmGmU*mC*mA |
| D: | SEQ ID NO: 798 | mG*mUmGmAmAmAfGmGfAfAfAmGmCmUmAmGmGmGmA*mC*mA |
| 347 | SEQ ID NO: 799 | mU*fG*mUmCmCfCmUmAmGmCmUmUmUfCmCfUmUmUmCmAmC*mC*mU |
| D: | SEQ ID NO: 800 | mA*mG*mCmUmCmUfUmUfCfAfCmUmUmCmGmGmAmGmG*mA*mA |
| 348 | SEQ ID NO: 801 | mU*fU*mCmCmUfCmCmGmAmAmGmUmGfAmAfAmGmAmGmCmU*mU*mC |
| D: | SEQ ID NO: 802 | mU*mU*mCmUmUmCfCmAfAfAfUmGmCmAmGmAmAmGmA*mU*mA |
| 349 | SEQ ID NO: 803 | mU*fA*mUmCmUfUmCmUmGmCmAmUmUfUmGfGmAmAmGmAmA*mA*mA |
| D: | SEQ ID NO: 804 | mG*mG*mAmGmGmAfUmUfGfCfUmCmAmAmCmAmAmCmC*mA*mA |
| 350 | SEQ ID NO: 805 | mU*fU*mGmGmUfUmGmUmUmGmAmGmCfAmAfUmCmCmUmCmC*mG*mA |
| D: | SEQ ID NO: 806 | mU*mG*mCmCmAmAfGmAfAfGfGmGmAmAmGmGmAmGmU*mA*mA |
| 351 | SEQ ID NO: 807 | mU*fU*mAmCmUfCmCmUmUmCmCmCmUfUmCfUmUmGmGmCmA*mG*mG |
| D: | SEQ ID NO: 808 | mG*mA*mAmAmGmUfUmCFA-(AP)-fCmUmGmCmUmUmCmGmU*mA*mA |
| 352 | SEQ ID NO: 562 | mU*U*mAmCmGmAmAmGmCmAmGmUmUfGmAfAmCmUmUmUmC*mU*mG |
| D: | SEQ ID NO: 808 | mG*mA*mAmAmGmUfUmCFA-(AP)-fCmUmGmCmUmUmCmGmU*mA*mA |
| 353 | SEQ ID NO: 563 | mU*fU*mAmCfGmAfAmGmCmAmGmUmUfGmAfAmCmUmUmUmC*mU*mG |
| D: | SEQ ID NO: 808 | mG*mA*mAmAmGmUfUmCfA-(AP)-fCmUmGmCmUmUmCmGmU*mA*mA |
| 354 | SEQ ID NO: 571 | mU*fU*fAmCmGmAfAmGmCmAmGmUmUfGmAfAmCmUmUmUmC*mU*mG |
| D: | SEQ ID NO: 809 | mG*mA*mAmAmGmUmUmCfAfAfCmUmGmCmUmUmCmGmU*mA*mA |
| 355 | SEQ ID NO: 563 | mU*fU*mAmCfGmAfAmGmCmAmGmUmUfGmAfAmCmUmUmUmC*mU*mG |
| D: | SEQ ID NO: 809 | mG*mA*mAmAmGmUmUmCfAfAfCmUmGmCmUmUmCmGmU*mA*mA |
| 356 | SEQ ID NO: 571 | mU*fU*fAmCmGmAfAmGmCmAmGmUmUfGmAfAmCmUmUmUmC*mU*mG |
| D: | SEQ ID NO: 810 | mG*mU*mUmGmAmCfUmUfG-(AP)-fGmUmAmAmAmUmAmUmA*mU*mA |
| 357 | SEQ ID NO: 566 | mU*fA*mUmAfUmAfUmUmUmAmCmUmCfAmAfGmUmCmAmAmC*mA*mU |
| D: | SEQ ID NO: 810 | mG*mUmUmGmAmCfUmUfG-(AP )-fGmUmAmAmAmUmAmUmA*mU*mA |
| 358 | SEQ ID NO: 567 | mU*A*fUmAmUmAfUmUmUmAmCmUmCfAmAfGmUmCmAmAmC*mA*mU |
| D: | SEQ ID NO: 811 | mG*mU*mUmGmAmCmUmUfGfAfGmUmAmAmAmUmAmUmA*mU*mA |
| 359 | SEQ ID NO: 566 | mU*fA*mUmAfUmAfUmUmUmAmCmUmCfAmAfGmUmCmAmAmC*mA*mU |
| D: | SEQ ID NO: 811 | mG*mU*mUmGmAmCmUmUfGfAfGmUmAmAmAmUmAmUmA*mU*mA |
| 360 | SEQ ID NO: 567 | mU*A*fUmAmUmAfUmUmUmAmCmUmCfAmAfGmUmCmAmAmC*mA*mU |
| D: | SEQ ID NO: 592 | mG*mUmUmGmAmCfUmUfGfAfGmUmAmAmAmUmAmUmA*mU*mA |
| 361 | SEQ ID NO: 569 | mU*fA*mUmAfU-GNA(A)- |
| | | fUmUmUmAmCmUmCfAmAfGmUmCmAmAmC*mA*mU |
| D: | SEQ ID NO: 592 | mG*mU*mUmGmAmCfUmUfGfAfGmUmAmAmAmUmAmUmA*mU*mA |
| 362 | SEQ ID NO: 570 | mU*fA*mUmAmUfA-GNA (U)- |
| | | mUmUmAmCmUmCfAmAfGmUmCmAmAmC*mA*mU |
| D: | SEQ ID NO: 812 | mA*mG*mAmAmAmGfUmUfCfAfAmCmUmGmCmUmUmCmG*mU*mA |
| 363 | SEQ ID NO: 813 | mU*fA*mCmGmAfAmGmCmAmGmUmUmGfAmAfCmUmUmUmCmU*mG*mU |
| D: | SEQ ID NO: 814 | mC *mU*mGmUmAmUmGmUfGfAfAmCmAmCmUmGmUmGmA*mC*mA |
| 364 | SEQ ID NO: 815 | mU*fG*fUmCmAmCfAmGmUmGmUmUmCfAmCfAmUmAmCmAmG*mU*mA |
| D: | SEQ ID NO: 816 | mU*mG*mAmCmUmUmGmAfGfUfAmAmAmUmAmUmAmUmC*mA*mA |
| 365 | SEQ ID NO: 817 | mU*fU*fGmAmUmAfUmAmUmUmUmAmCfUmCfAmAmGmUmCmA*mA*mC |
| D: | SEQ ID NO: 818 | mG*mU*mAmUmGmUmGmAfAfCfAmCmUmGmUmGmAmCmC*mC*mA |
| 366 | SEQ ID NO: 819 | mU*fG*fGmGmUmCfAmCmAmGmUmGmUfUmCfAmCmAmUmAmC*mA*mG |

m indicates 2′O-methyl modified ribose on the listed nucleotide;
f indicates 2′F modified ribose on the listed nucleotide;
*indicates a phosphorothioate bond (in place of a phosphodiester bond);
GNA indicates a glycol nucleic acid nucleotide;
(AP) means an apurinic/apyrimidinic residue, also called an abasic residue.

TABLE 5
Percent Inhibition of human FAS expression in HepG2 cells

| | NV-hFAS HepG2 qPCR | | NV-hFAS HepG2 qPCR | |
|---|---|---|---|---|
| | siRNA Knockdown (% | | siRNA Knockdown | |
| | inhibition) Single | | Concentration Response | |
| | point Summary | | Curve Summary | |
| | | Conc. | Rel IC50 | |
| Duplex | % Inh | (nM) | (nM) | % pct Rel Max |
| D: 112 | 95.1 | 500 | 0.372 | 93.1 |
| D: 113 | 93.5 | 500 | 0.425 | 92 |
| D: 114 | 92.8 | 500 | 0.847 | 92.8 |
| D: 115 | 92.8 | 500 | 0.551 | 92.4 |
| D: 116 | 92.6 | 500 | 0.517 | 92.7 |
| D: 117 | 92.1 | 500 | 0.274 | 93.9 |
| D: 118 | 91.9 | 500 | 0.854 | 90.4 |
| D: 119 | 91.5 | 500 | 0.311 | 91.9 |
| D: 120 | 91 | 500 | 0.486 | 74.8 |
| D: 121 | 89.6 | 500 | 0.441 | 91.3 |
| D: 122 | 89.5 | 500 | 0.4 | 84.2 |
| D: 123 | 89.5 | 500 | 0.969 | 88.2 |
| D: 124 | 89.1 | 500 | 0.49 | 93.4 |
| D: 125 | 88.6 | 500 | 0.908 | 82.9 |
| D: 126 | 88.1 | 500 | | |
| D: 127 | 87.9 | 500 | 0.311 | 89.1 |
| D: 128 | 87.8 | 500 | 0.274 | 89.6 |
| D: 129 | 87.4 | 500 | 0.78 | 78.5 |
| D: 130 | 87.1 | 500 | 0.766 | 86.8 |
| D: 131 | 87 | 500 | 1.35 | 86.4 |
| D: 132 | 87 | 500 | 0.488 | 77.9 |
| D: 133 | 87 | 500 | 0.856 | 90.7 |
| D: 134 | 86.9 | 500 | | |
| D: 135 | 86.5 | 500 | 0.642 | 85.8 |
| D: 136 | 86.3 | 500 | 0.55 | 82.4 |
| D: 137 | 85.4 | 500 | 0.511 | 89.6 |
| D: 138 | 85.3 | 500 | 0.702 | 70.4 |
| D: 139 | 85.1 | 500 | 0.401 | 88.1 |
| D: 140 | 85.1 | 500 | 1.12 | 89.8 |
| D: 141 | 84.8 | 500 | 0.537 | 91.1 |
| D: 142 | 84.4 | 500 | 0.989 | 84.2 |
| D: 143 | 84.2 | 500 | 0.733 | 77.9 |
| D: 144 | 84.1 | 500 | | |
| D: 145 | 84 | 500 | 0.76 | 86.9 |
| D: 146 | 83.6 | 500 | 0.426 | 90.4 |
| D: 147 | 83.2 | 500 | 0.446 | 88.2 |
| D: 148 | 83 | 500 | 0.933 | 80.3 |
| D: 149 | 82.6 | 500 | 0.855 | 86.7 |
| D: 150 | 82.4 | 500 | 0.705 | 88.7 |
| D: 151 | 81.8 | 500 | 0.849 | 84.5 |
| D: 152 | 81.7 | 500 | 0.611 | 86.8 |
| D: 153 | 81.4 | 500 | 0.505 | 85 |
| D: 154 | 80.8 | 500 | 0.519 | 83.4 |
| D: 155 | 80.7 | 500 | | |
| D: 156 | 80.6 | 500 | 1.2 | 82.8 |
| D: 157 | 80.4 | 500 | 2.32 | 77.6 |
| D: 158 | 80.1 | 500 | | |
| D: 159 | 80.1 | 500 | 0.388 | 79.4 |
| D: 160 | 80 | 500 | | |
| D: 161 | 79.9 | 500 | 0.38 | 83.2 |
| D: 162 | 79.7 | 500 | 1.01 | 78.7 |
| D: 163 | 79.3 | 500 | | |
| D: 164 | 78.8 | 500 | 0.913 | 76 |
| D: 165 | 78.6 | 500 | | |
| D: 166 | 78 | 500 | | |
| D: 167 | 77.9 | 500 | 1.63 | 67.3 |
| D: 168 | 76.8 | 500 | | |
| D: 169 | 76.6 | 500 | | |
| D: 170 | 76.5 | 500 | | |
| D: 171 | 76.4 | 500 | | |
| D: 172 | 76.4 | 500 | | |
| D: 173 | 75.7 | 500 | 1.35 | 80.3 |
| D: 174 | 75.2 | 500 | | |
| D: 175 | 74.9 | 500 | | |
| D: 176 | 74.9 | 500 | | |
| D: 177 | 74.9 | 500 | | |
| D: 178 | 74.3 | 500 | | |
| D: 179 | 73.9 | 500 | | |
| D: 180 | 72.3 | 500 | | |
| D: 181 | 72.2 | 500 | | |
| D: 182 | 71.9 | 500 | | |
| D: 183 | 71.8 | 500 | | |
| D: 184 | 71.8 | 500 | | |
| D: 185 | 71.2 | 500 | | |
| D: 186 | 71.1 | 500 | | |
| D: 187 | 71.1 | 500 | | |
| D: 188 | 70.8 | 500 | | |
| D: 189 | 70.7 | 500 | | |
| D: 190 | 70.5 | 500 | | |
| D: 191 | 70 | 500 | | |
| D: 192 | 70 | 500 | | |
| D: 193 | 69.3 | 500 | | |
| D: 194 | 68.4 | 500 | | |
| D: 195 | 67.1 | 500 | | |
| D: 196 | 66.5 | 500 | | |
| D: 197 | 66 | 500 | | |
| D: 198 | 65.5 | 500 | | |
| D: 199 | 65 | 500 | | |
| D: 200 | 64.5 | 500 | | |
| D: 201 | 64.5 | 500 | | |
| D: 202 | 63.6 | 500 | | |
| D: 203 | 63.5 | 500 | | |
| D: 204 | 63.5 | 500 | | |
| D: 205 | 63.4 | 500 | | |
| D: 206 | 61.1 | 500 | | |
| D: 207 | 61 | 500 | | |
| D: 208 | 60.7 | 500 | | |
| D: 209 | 59.8 | 500 | | |
| D: 210 | 59.3 | 500 | | |
| D: 211 | 58.4 | 500 | | |
| D: 212 | 58.4 | 500 | | |
| D: 213 | 57.8 | 500 | | |
| D: 214 | 55.8 | 500 | | |
| D: 215 | 55.1 | 500 | | |
| D: 216 | 54 | 500 | | |
| D: 217 | 53.6 | 500 | | |
| D: 218 | 52.5 | 500 | | |
| D: 219 | 52 | 500 | | |
| D: 220 | 51.4 | 500 | | |
| D: 221 | 50.1 | 500 | | |
| D: 222 | 50 | 500 | | |
| D: 223 | | | 0.281 (0.192, | 57.3 (7.22, n = 2) |
| | | | n = 2) | |
| D: 224 | | | 0.417 (0.0001, | 80.9 (1.16, n = 2) |
| | | | n = 2) | |
| D: 225 | | | 0.375 (0.0255, | 77.7 (2.09, n = 2) |
| | | | n = 2) | |
| D: 226 | | | 0.121 (0.0048, | 82.9 (2.92, n = 2) |
| | | | n = 2) | |
| D: 227 | | | 0.0774 (0.0472, | 80.4 (1.43, n = 2) |
| | | | n = 2) | |
| D: 228 | | | 0.469 | 90.6 |
| D: 229 | | | 0.325 | 88.5 |
| D: 230 | | | 0.231 | 94.2 |
| D: 231 | | | 0.0324 | 91.4 |
| D: 232 | | | 0.0895 | 90.2 |
| D: 233 | | | 0.448 | 81.9 |
| D: 238 | | | 0.095 | 94 |
| D: 239 | | | 0.129 | 93.9 |
| D: 227 | | | 0.031 | 80 |
| D: 231 | | | 0.045 | 93.9 |
| D: 240 | | | 0.535 | 95.2 |

TABLE 6A
In vivo FAS mRNA Knockdown (% KD) and remaining FAS
protein in RNAi agent treated mice expressing hFAS

| | In vivo RNAi treatment in AAV-hFAS Mice | | | | | |
|---|---|---|---|---|---|---|
| | | Run 1 | Run 2 | Run 3 | Run 4 | |
| | 10 wk, | 10 wk, | 10 wk, | 3 wk, | 3 wk, | 2 wk, |
| | 1 mpk, | 3 mpk, | 3 mpk, | 3 mpk, | 3 mpk, | 5 mpk, |
| | % huFas | % huFas | % huFas | % huFas | % huFas | % huFas |
| | mRNA | mRNA | mRNA | mRNA | mRNA | protein |
| Duplex* | KD | KD | KD | KD | KD | remaining |
| D: 117 | | | | | | 66 |
| D: 112 | | | | | | 34 |
| D: 124 | | | | | | 54 |
| D: 116 | 2.4 | 20.2 | | | | 67 |
| D: 114 | 17.8 | 26.3 | | | | 68 |
| D: 115 | 6.3 | 51 | 51 | | | 83 |
| D: 113 | 17 | 11.5 | | | | 70 |
| D: 119 | | | | | | 61 |
| D: 133 | | | | | | 40 |
| D: 121 | | | | | | 64 |
| D: 141 | 20.8 | 29.3 | | | | 67 |
| D: 137 | | | | | | 50 |
| D: 146 | | | | | | 42 |
| D: 128 | 10.9 | 44.2 | | 71.3 | 52.8 | 91 |
| D: 140 | | | | | | 54 |
| D: 118 | | | | | | 70 |
| D: 150 | 44.7 | 35.1 | | | | 70 |
| D: 127 | 26.2 | 52 | | | | 78 |
| D: 147 | | | | | | 56 |
| D: 139 | | | | | | 39 |
| D: 223 | | | 24.6 | | | |
| D: 224 | | | 25.3 | | | |
| D: 225 | | | 48.4 | | | |
| D: 226 | | | 39.1 | | | |
| D: 227 | | | 47.3 | | | |
| D: 228 | | | 1.7 | | | |
| D: 229 | | | 21.7 | | | |
| D: 230 | | | 42.2 | | | |
| D: 231 | | | 49.4 | | | |
| D: 232 | | | 16.1 | | | |
| D: 233 | | | 5.8 | | | |
| D: 172 | | | | 16 | | |
| D: 162 | | | | | 11.7 | |
| D: 151 | | | | 36.7 | | |
| D: 130 | | | | | 38.2 | |
| D: 154 | | | | | 60.8 | |
| D: 158 | | | | | 26.3 | |

*for all duplexes/RNAi agents tested via administration to mice, the antisense strand does not have an extra phosphate addition as shown in Table 4A.

TABLE 6B
In vivo FAS mRNA Knockdown (% KD) in RNAi
agent treated mice expressing hFAS

| | 2 weeks | | | 6 weeks | | |
|---|---|---|---|---|---|---|
| | 1 mpk, | 3 mpk, | 10 mpk, | 1 mpk, | 3 mpk, | 10 mpk, |
| | % huFas | % huFas | % huFas | % huFas | % huFas | % huFas |
| Duplex | KD | KD | KD | KD | KD | KD |
| D: 238 | 50.3 | 71.6 | 95 | 44.7 | 60.2 | 85.4 |
| D: 239 | 41.3 | 62.5 | 89.5 | 13 | 59.6 | 66.3 |
| D: 227 | 54.2 | 81.5 | 94.6 | 22.1 | 60.9 | 91.8 |
| D: 231 | 46.4 | 80.2 | 93.5 | 4 | 50.4 | 80.9 |

### Example 6

### Protein Determination In Vivo in AAV-Fas Expressing Mice Treated with RNAi Agents as Shown in Table 6A and 6B

Liver samples from the above RNAi agent treated mice were snap frozen and stored at −80 C. While frozen, the lysing matrix D tubes containing ~⅓ liver were transferred to wet ice and XY lite containing 2× Halt buffer was added to each sample at 700 ul/tube. Samples were homogenized using Fast Prep 96 at 1800 rpm for 60 seconds and cooled on ice for 5 minutes. The process was then repeated for another 30 second round of homogenization and then spun down at 20,000 rcf for 5 minutes at 4 C. Samples were centrifuged at 20,000 ref for 10 min at 4 C in Eppendorf tubes to remove cell debris. Protein quantitation was performed on supernatants using the following procedure.

All samples were equilibrated to 2.0 mg/ml. Supernatants (in XY buffer) were aliquoted into 2 96-well plates at 100 ul sample per well and stored at −80 C and subject to protein quantitation. Prepare BSA standard was prepared at 2 mg/ml and diluted in lysis buffer to create standards. Samples were diluted 1:50 by adding 2 ul lysate to 98 ul XY lite and HALT in a 96 well plate (Corning #3790) and mixed by pipetting. Next 3 mLs Biorad Reagent A and 60 ul of Biorad Reagent S were combined to make Reagent C. 25 ul of Reagent C was added to each well in 96 well plate (Corning #3596). Next, 5 uL of standards or diluted sample was added to each well of the 96 well plate containing reagent C and performed in duplicate. Absorbance was read at 750 nm on SpectraMax in 77/3/350.

The quantified supernatants were then subject to an Elisa using the Human FAS DuoSet ELISA protocol. Capture antibody was diluted to the working concentration (1.0 ug/ml) in PBS. Immediately, 100 ul was added per well and incubated overnight at room temperature. The next day, plate wells were decanted and washed 3 times with 300 uL/well wash buffer. Plates were blocked by adding 300 ul/well Reagent diluent to each well and incubated at room temperature for 1 hour. The wells were decanted and washed 3 times with 300 ul/well 1× wash buffer and blotted dry after the final wash. Standard curve with control FAS protein were made by diluting in reagent diluent to final concentrations of 4000, 2000, 1000, 500, 250, 125, 62.5, or 0 pg/ml. Thawed liver lysates or standards were added per well and the plate was sealed and incubated for 2 hours at room temperature with gentle shaking. Samples were added at 100 ul/well at 0.1 ug/ul for 10 ug total protein/well diluted in reagent diluent. After incubation, assay plate was decanted and washed 3 times with 300 ul/well 1× wash buffer and blotted dry. 100 ul/well of the detection antibody diluted in reagent diluent was added. The plate was sealed and incubated for 2 hours at room temperature. The detection antibody was diluted to working concentration of 50 ng/ml with reagent diluent. After incubation, assay plate was decanted and washed 3 times with 300 ul/well 1× wash buffer and blotted dry. 100 ul/well of the working dilution (1:200) of Strep-HRP diluted in reagent diluent was added to the plate. The plate was covered and incubated for 20 minutes at room temperature, protecting the plate from direct light. After incubation, the assay plate was decanted, washed 3 times and blotted dry. Next 100 ul/well of Substrate Solution was added and the plate incubated for 20 minutes at room temperature, protecting from direct light. Stop Solution (50 uL) was added to each well and gently mixed. The OD of each well was measured within 30 minutes of adding stop solution using a SpectraMax in 77/3/350 at 450 nm with correction of 540 nm (OD@450 nm-OD@540 nm).

Results are shown in Tables 6A and 6B.

### Example 7: Additional RNAi Agent Tested for Knockdown

The additional FAS-GalNAc RNAi agent D-235 shown in Table 7 below is tested in vitro in HepG2 cells as described above and shows about 50% or greater knockdown as compared to a vehicle control. The RNAi agent is tested in AAV-hFAS treated mice, for mRNA knockdown and protein knockdown as described above.

TABLE 7
Additional Sequences

| | | Sequence-Sense Strand |
|---|---|---|
| | | (top) 5′ to 3′ |
| Duplex | SEQ ID | Antisense strand |
| No: | NO: | (bottom) 5′ to 3′ |
| D: 234 | SEQ ID | AGAAAGUUCAACUGCUUCGUA |
| | NO: 572 | |
| | SEQ ID | UACGAAGCAGUUGAACUUUCUGU |
| | NO: 573 | |
| D: 235 | SEQ ID | mA*mG*mAmAmAmGfUmUfCfAf |
| | NO: 574 | AmCmUmGmCmUmUmCmG*mU*mA |
| | SEQ ID | mU*fA*mCmGmAfAmGmCmAmGmU |
| | NO: 575 | mUmGfAmAfCmUmUmUmCmU*mG*mU |

RNAi agents to mouse FAS mRNA were also generated and tested (see Table 8).

TABLE 8
RNAi agent to mouse FAS mRNA

| | | Sequence-Sense Strand |
|---|---|---|
| | | (top) 5′ to 3′ |
| | SEQ ID | Antisense strand |
| Duplex | NO: | (bottom) 5′ to 3′ |
| D: 367 | SEQ ID | GCCGAAUGUCGCAGAACCUUA |
| | NO: 820 | |
| | SEQ ID | UAAGGUUCUGCGACAUUCGGCUU |
| | NO: 821 | |
| D: 368 | SEQ ID | CCGAAUGUCGCAGAACCUUAA |
| | NO: 822 | |
| | SEQ ID | UUAAGGUUCUGCGACAUUCGGCU |
| | NO: 823 | |
| D: 369 | SEQ ID | AUGUCGCAGAACCUUAGAUAA |
| | NO: 824 | |
| | SEQ ID | UUAUCUAAGGUUCUGCGACAUUC |
| | NO: 825 | |
| D: 370 | SEQ ID | mG*mC*mCmGmAmAmUmGfUfCfGm |
| | NO: 826 | CmAmGmAmAmCmCmU*mU*mA |
| | SEQ ID | mU*fA*fAmGmGmUfUmCmUmGmCm |
| | NO: 827 | GmAfCmAfUmUmCmGmGmC*mU*mU |
| D: 371 | SEQ ID | mC*mC*mGmAmAmUmGmUfCfGfCm |
| | NO: 828 | AmGmAmAmCmCmUmU*mA*mA |
| | SEQ ID | mU*fU*fAmAmGmGfUmUmCmUmGm |
| | NO: 829 | CmGfAmCfAmUmUmCmGmG*mC*mU |
| D: 372 | SEQ ID | mA*mU*mGmUmCmGmCmAfGfAfAm |
| | NO: 830 | CmCmUmUmAmGmAmU*mA*mA |
| | SEQ ID | mU*fU*fAmUmCmUfAmAmGmGmUm |
| | NO: 831 | UmCfUmGfCmGmAmCmAmU*mU*mC |

### Example 8. Characterization of FAS RNAi Agent in Cynomolgus Monkey

In vivo testing of selected FAS RNAi agents in Cynomolgus monkey (Macaca fascicularis) was conducted to assess their efficacy in silencing the target gene in liver. Cynomolgus monkeys (n=3/group) were given a single subcutaneous administration of the FAS RNAi agent (3 mg/kg, 0.5 ml/kg, in sterile 1×PBS (pH 7.2)) or sterile 1×PBS (pH 7.2) (0.5 ml/kg). Following administration of FAS RNAi agent, incisional wedge biopsies of the liver were collected at 28 days post-administration. cDNA was prepared from RNA isolated from monkey liver samples, and qPCR was performed to determine FAS mRNA knockdown. Table 9 shows the mRNA knockdown of FAS expression in liver at 28 days after the administration of the FAS RNAi agent compared to PBS control group.

TABLE 9
FAS mRNA knockdown in cynomolgus monkey.

| | 28 days |
|---|---|
| | % huFas mRNA KD |
| Duplex | (3 mg/kg) |
| D: 238 | 47 |
| D: 227 | 72 |
| D: 240 | 51 |

1. An RNAi agent for reducing FAS gene expression, wherein the RNAi agent comprises a delivery moiety of Formula I conjugated to R, wherein R is a double stranded RNA (dsRNA) comprising an antisense strand and a sense strand: 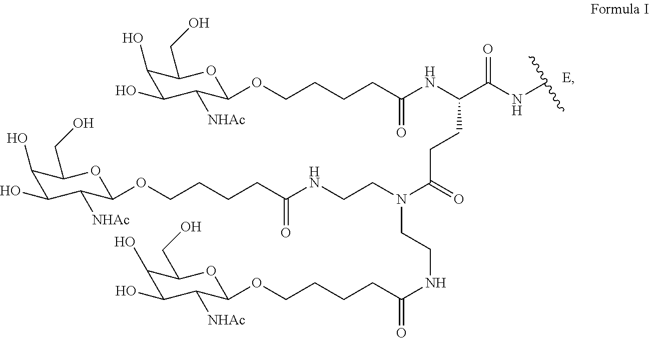
wherein R is conjugated to connection point E of Formula I, optionally via a linker,
wherein the sense strand and the antisense strand form a duplex region, and
wherein the antisense strand comprises a region of complementarity to a FAS mRNA target sequence of SEQ ID NO: 1, and wherein the sense and antisense strand each optionally comprise one or more modified nucleotides and one or more modified internucleotide linkages.

2. The RNAi agent of claim 1, wherein Formula I is conjugated to the sense strand, optionally via a linker.

3. The RNAi agent of claim 2, wherein Formula I is conjugated to the 3′ terminal nucleotide of the sense strand, optionally via a linker.

4. The RNAi agent of claim 1, wherein the antisense strand is 15 to 50 nucleotides in length.

5. The RNAi agent of claim 1, wherein the sense strand is 15 to 50 nucleotides in length.

6. The RNAi agent of claim 1, wherein the antisense strand is between 18 and 23 nucleotides in length.

7. The RNAi agent of claim 1, wherein the sense strand is between 18 and 21 nucleotides in length.

8. The RNAi agent of claim 1, wherein the antisense strand is 23 nucleotides in length and the sense strand is 21 nucleotides in length.

9. The RNAi agent of claim 1, wherein the region of complementarity is at least 18 nucleotides in length.

10. The RNAi agent of claim 1, wherein the antisense strand comprises a sequence selected from the group consisting of SEQ ID NO: 2 to SEQ ID NO: 112.

11. The RNAi agent of claim 1, wherein the antisense strand comprises at least 18 contiguous nucleotides of a sequence selected from the group consisting of SEQ ID NOs: 224 to 334, 337, 338, 573, and 577.

12. The RNAi agent of claim 1, wherein the antisense strand has a nucleotide sequence selected from the group consisting of SEQ ID NOs: 224-334, 337, 338, 573, and 577, or a sequence having at least 90% sequence identity thereto.

13. The RNAi agent of claim 1, wherein the sense strand is selected from the group consisting of SEQ ID NOs: 113 to 223, 335, 336, 572, and 576, or a sequence having at least 90% sequence identity thereto.

14. The RNAi agent of claim 1, wherein the duplex region between the sense strand and the antisense strand comprises 0, 1, 2, or 3 mismatches between the sense strand and the antisense strand.

15. The RNAi agent of claim 1, wherein the sense strand comprises a first nucleic acid sequence and the antisense strand comprises a second nucleic acid sequence, wherein the first nucleic acid sequence and the second nucleic acid sequence are selected from the group consisting of:
a. the first nucleic acid sequence has at least 95% sequence identity to SEQ ID NO: 129, and the second nucleic acid sequence has at least 95% sequence identity to SEQ ID NO: 240;
b. the first nucleic acid sequence has at least 95% sequence identity to SEQ ID NO: 116, and the second nucleic acid sequence has at least 95% sequence identity to SEQ ID NO: 227;
c. the first nucleic acid sequence has at least 95% sequence identity to SEQ ID NO: 151, and the second nucleic acid sequence has at least 95% sequence identity to SEQ ID NO: 262;
d. the first nucleic acid sequence has at least 95% sequence identity to SEQ ID NO: 128, and the second nucleic acid sequence has at least 95% sequence identity to SEQ ID NO: 239; and
e. the first nucleic acid sequence has at least 95% sequence identity to SEQ ID NO: 155, and the second nucleic acid sequence has at least 95% sequence identity to SEQ ID NO: 266.

16. The RNAi agent of claim 1, wherein the sense strand comprises a first nucleic acid sequence and the antisense strand comprises a second nucleic acid sequence, wherein the first nucleic acid sequence and the second nucleic acid sequence are selected from the group consisting of:
a. the first nucleic acid sequence comprises SEQ ID NO: 129, and the second nucleic acid sequence comprises SEQ ID NO: 240;
b. the first nucleic acid sequence comprises SEQ ID NO: 116, and the second nucleic acid sequence comprises SEQ ID NO: 227;
c. the first nucleic acid sequence comprises SEQ ID NO: 151, and the second nucleic acid sequence comprises SEQ ID NO: 262;
d. the first nucleic acid sequence comprises SEQ ID NO: 128, and the second nucleic acid sequence comprises SEQ ID NO: 239; and
e. the first nucleic acid sequence comprises SEQ ID NO: 155, and the second nucleic acid sequence comprises SEQ ID NO: 266.

17. The RNAi agent of claim 16, wherein the sense strand or the antisense strand each independently comprise one or more modified nucleotides.

18. The RNAi agent of claim 17, wherein the sense strand or the antisense strand each independently comprise one or more modified nucleotides, and the modified nucleotides are independently 2′ fluoro modified nucleotide residues, 2′-O-methyl modified nucleotides, or glycol nucleic acid (GNA) nucleotides.

19. The RNAi agent of claim 18, wherein the sense strand comprises one or more modified nucleotide residues, and wherein at least one modified nucleotide residue is a GNA nucleotide that is present in an internal position of the sense strand.

20. The RNAi agent of claim 19, wherein each nucleotide of the sense strand and each nucleotide of the antisense strand is a modified nucleotide.

21. The RNAi agent of claim 20, wherein the antisense strand is 23 nucleotides in length and wherein each nucleotide of the antisense strand is a modified nucleotide, and 2′ fluoro modified nucleotides are present at
a. Positions 2, 3, 7, 14, and 16 from the 5′ end of the antisense strand; or
b. Positions 2, 5, 7, 14, and 16 from the 5′ end of the antisense strand; or
c. Positions 2, 3, 8, 14, and 16 from the 5′ end of the antisense strand; or
d. Positions 2, 5, 8, 14, and 16 from the 5′ end of the antisense strand; or
e. Positions 2, 6, 14, and 16 from the 5′ end of the antisense strand.

22. The RNAi agent of claim 21, wherein the sense strand and antisense strand each independently comprise one or more modified internucleotide linkages, and wherein each modified internucleotide linkage is a phosphorothioate linkage.

23. The RNAi agent of claim 22, wherein the sense strand and antisense strand each independently comprise four phosphorothioate linkages.

24. The RNAi agent of claim 22, wherein the 5′ terminal nucleotide of the antisense strand comprises a phosphate group or a phosphate analog.

25. The RNAi agent of claim 1, wherein the antisense strand comprises a sequence selected from the group consisting of SEQ ID NOs: 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 563, 566, 567, 569, 570, 571, 575, 579, 581, 583, 585, or a sequence having at least 90% sequence identity thereto, wherein the 5′ terminal nucleotide of the antisense strand comprises a vinyl phosphonate, a phosphate, or a hydroxyl group.

26. The RNAi agent of claim 1, wherein the sense strand comprises a sequence selected from the group consisting of SEQ ID NOs: 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 564, 565, 568, 574, 578, 580, 582, 584 or a sequence having at least 90% sequence identity thereto, wherein the 5′ terminal nucleotide of the antisense strand comprises a vinyl phosphonate, a phosphate, or a hydroxyl group.

27. The RNAi agent of claim 1, wherein the antisense strand comprises a sequence selected from the group consisting of SEQ ID NOs: 587, 589, 591, 593, 595, 597, 599, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 663, 665, 667, 669, 671, 673, 675, 677, 679, 681, 683, 685, 687, 689, 691, 693, 695, 697, 699, 701, 703, 705, 707, 709, 711, 713, 715, 717, 719, 721, 723, 725, 727, 729, 731, 733, 735, 737, 739, 741, 743, 745, 747, 749, 751, 753, 755, 757, 759, 761, 763, 765, 767, 769, 771, 773, 775, 777, 779, 781, 783, 785, 787, 789, 791, 793, 795, 797, 799, 801, 803, 805, 807, 813, 815, 817, 819, or a sequence having at least 90% sequence identity thereto.

28. The RNAi agent of claim 1, wherein the sense strand comprises a sequence selected from the group consisting of SEQ ID NOs: 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 678, 680, 682, 684, 686, 688, 690, 692, 694, 696, 698, 700, 702, 704, 706, 708, 710, 712, 714, 716, 718, 720, 722, 724, 726, 728, 730, 732, 734, 736, 738, 740, 742, 744, 746, 748, 750, 752, 754, 756, 758, 760, 762, 764, 766, 768, 770, 772, 774, 776, 778, 780, 782, 784, 786, 788, 790, 792, 794, 796, 798, 800, 802, 804, 806, 808, 809, 810, 811, 812, 814, 816, 818, or a sequence having at least 90% sequence identity thereto.

29. The RNAi agent of claim 1, wherein the sense strand comprises a first nucleic acid sequence and the antisense strand comprises a second nucleic acid sequence, wherein the first nucleic acid sequence and the second nucleic acid sequence are selected from the group consisting of:
a. the first nucleic acid sequence has at least 95% sequence identity to SEQ ID NO: 339, and the second nucleic acid sequence has at least 95% sequence identity to SEQ ID NO: 340;
b. the first nucleic acid sequence has at least 95% sequence identity to SEQ ID NO: 341, and the second nucleic acid sequence has at least 95% sequence identity to SEQ ID NO: 342;
c. the first nucleic acid sequence has at least 95% sequence identity to SEQ ID NO: 343, and the second nucleic acid sequence has at least 95% sequence identity to SEQ ID NO: 344;
d. the first nucleic acid sequence has at least 95% sequence identity to SEQ ID NO:345, and the second nucleic acid sequence has at least 95% sequence identity to SEQ ID NO: 346;
e. the first nucleic acid sequence has at least 95% sequence identity to SEQ ID NO: 347, and the second nucleic acid sequence has at least 95% sequence identity to SEQ ID NO: 348;
f. the first nucleic acid sequence has at least 95% sequence identity to SEQ ID NO: 349, and the second nucleic acid sequence has at least 95% sequence identity to SEQ ID NO: 350;
g. the first nucleic acid sequence has at least 95% sequence identity to SEQ ID NO: 353, and the second nucleic acid sequence has at least 95% sequence identity to SEQ ID NO: 354;
h. the first nucleic acid sequence has at least 95% sequence identity to SEQ ID NO: 363 and the second nucleic acid sequence has at least 95% sequence identity to SEQ ID NO: 364; and
i. the first nucleic acid sequence has at least 95% sequence identity to SEQ ID NO: 381, and the second nucleic acid sequence has at least 95% sequence identity to SEQ ID NO: 382.

30. The RNAi agent of claim 1, wherein the sense strand comprises a first nucleic acid sequence and the antisense strand comprises a second nucleic acid sequence, wherein the first nucleic acid sequence and the second nucleic acid sequence are selected from the group consisting of:
a. the first nucleic acid sequence has at least 95% sequence identity to SEQ ID NO: 564 or 809, and the second nucleic acid sequence has at least 95% sequence identity to SEQ ID NO: 571;
b. the first nucleic acid sequence has at least 95% sequence identity to SEQ ID NO: 568 or 811, and the second nucleic acid sequence has at least 95% sequence identity to SEQ ID NO: 567;
c. the first nucleic acid sequence has at least 95% sequence identity to SEQ ID NO: 580 or 814, and the second nucleic acid sequence has at least 95% sequence identity to SEQ ID NO: 581 or 815;
d. the first nucleic acid sequence has at least 95% sequence identity to SEQ ID NO: 582 or 816, and the second nucleic acid sequence has at least 95% sequence identity to SEQ ID NO: 583 or 817; and
e. the first nucleic acid sequence has at least 95% sequence identity to SEQ ID NO: 584 or 818, and the second nucleic acid sequence has at least 95% sequence identity to SEQ ID NO: 585 or 819.

31. The RNAi agent of claim 1, wherein the sense strand comprises a first nucleic acid sequence and the antisense strand comprises a second nucleic acid sequence, wherein the first nucleic acid sequence and the second nucleic acid sequence are selected from the group consisting of:
a. the first nucleic acid sequence comprises SEQ ID NO: 564 or 809, and the second nucleic acid sequence comprises SEQ ID NO: 571;
b. the first nucleic acid sequence comprises SEQ ID NO: 568 or 811, and the second nucleic acid sequence comprises SEQ ID NO: 567;
c. the first nucleic acid sequence comprises SEQ ID NO: 580 or 814, and the second nucleic acid sequence comprises SEQ ID NO: 581 or 815;
d. the first nucleic acid sequence comprises SEQ ID NO: 582 or 816, and the second nucleic acid sequence comprises SEQ ID NO: 583 or 817; and
e. the first nucleic acid sequence comprises SEQ ID NO: 584 or 818, and the second nucleic acid sequence comprises SEQ ID NO: 585 or 819.

32. The RNAi agent of claim 29, wherein the 5′ terminal nucleotide of the antisense strand contains a vinyl phosphonate, a phosphate group, or an OH group.

33. The RNAi agent of claim 1, wherein R is conjugated to Formula I via a linker.

34. The RNAi agent of claim 1, wherein R is conjugated to Formula I via a linker, and wherein linker comprises a linker of Formula II having connection points A and B or the linker comprises Formula III having connection points C and D, and wherein: 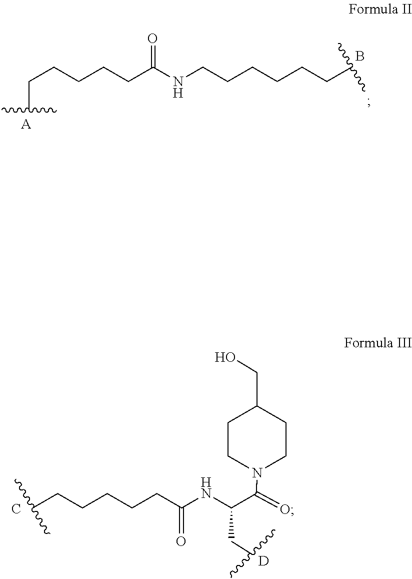
a. Formula I is conjugated to Formula II at connection point A and Formula II is conjugated to a phosphate group or a phosphorothioate group at connection point B, and the phosphate group or phosphorothioate group is further conjugated to R; or
b. Formula I is conjugated to Formula III at connection point C and Formula III is conjugated to a phosphate group or phosphorothioate group at connection point D, and the phosphate group or phosphorothioate group is further conjugated to R.

35. The RNAi agent of claim 1, wherein R is conjugated to Formula I via a linker, and wherein the linker is a linker comprising Formula III having connection points C and D: 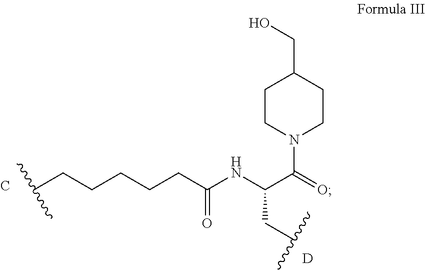
and wherein Formula I is conjugated to Formula III at connection point C and Formula III is conjugated to a phosphate group or a phosphorothioate group at connection point D, and the phosphate group or the phosphorothioate group is further conjugated to R.

36. The RNAi agent of claim 1, wherein the RNAi agent decreases expression of the FAS gene in a liver cell, as compared to a control.

37. The RNAi agent of claim 1, wherein the linker is of Formula III and Formula III is conjugated to a phosphate group at connection point D, and wherein the sense strand comprises a first nucleic acid sequence and the antisense strand comprises a second nucleic acid sequence, wherein the first nucleic acid sequence comprises SEQ ID NO: 564, and the second nucleic acid sequence comprises SEQ ID NO: 571.

38. (canceled)

39. A pharmaceutical composition comprising the RNAi agent of claim 1, and one or more pharmaceutically acceptable excipients.

40. (canceled)

41. A method of treating autoimmune hepatitis (AIH) in a patient in need thereof, comprising administering to the patient the RNAi agent of claim 1, or a pharmaceutical composition thereof.

42. A method of decreasing FAS expression in a cell, comprising contacting the cell with the RNAi agent of claim 1.

43. The method of claim 42, wherein the method further comprises incubating the cell for a time sufficient for decreasing the level of FAS mRNA by at least 50% as compared to an untreated or control treated cell.

44. A method of treating autoimmune hepatitis (AIH) in a patient in need thereof, comprising administering to the patient the RNAi agent of claim 37, or a pharmaceutical composition thereof.
